SEQUENCE LISTING (14 sequences)

SEQ ID NO: 1 - DNA, 993 bp, Homo sapien
atgacgcccaacagcactggcgaggtgcccagccccattcccaagggggctttggggctctccctggccctggcaagcctcatcatcaccgcgaacctgctcctagccctgggcatcgcctgggaccgccgcctgcgcagcccacctgctggctgcttcttcctgagcctactgctggctgggctgctcacgggtctggcattgcccacattgccagggctgtggaaccagagtcgccggggttactggtcctgcctcctcgtctacttggctcccaacttctccttcctctccctgcttgccaacctcttgctggtgcacggggagcgctacatggcagtcctgaggccactccagccccctgggagcattcggctggccctgctcctcacctgggctggtcccctgctctttgccagtctgcccgctctggggtggaaccactggacccctggtgccaactgcagctcccaggctatcttcccagccccctacctgtacctcgaagtctatgggctcctgctgcccgccgtgggtgctgctgccttcctctctgtccgcgtgctggccactgcccaccgccagctgcaggacatctgccggctggagcgggcagtgtgccgcgatgagccctccgccctggcccgggcccttacctggaggcaggcaagggcacaggctggagccatgctgctcttcgggctgtgctgggggccctacgtggccacactgctcctctcagtcctggcctatgagcagcgcccgccactggggcctgggacactgttgtccctcctctccctaggaagtgccagtgcagcggcagtgcccgtagccatggggctgggcgatcagcgctacacagccccctggagggcagccgcccaaaggtgcctgcaggggctgtggggaagagcctcccgggacagtcccggccccagcattgcctaccacccaagcagccaaagcagtgtcgacctggacttgaactaa

SEQ ID NO: 2 - protein, 330 aa, Homo sapien
MTPNSTGEVPSPIPKGALGLSLALASLIITANLLLALGIAWDRRLRSPPAGCFFLSLLLAGLLTGLALPTLPGLWNQSRRGYWSCLLVYLAPNFSFLSLLANLLLVHGERYMAVLRPLQPPGSIRLALLLTWAGPLLFASLPALGWNHWTPGANCSSQAIFPAPYLYLEVYGLLLPAVGAAAFLSVRVLATAHRQLQDICRLERAVCRDEPSALARALTWRQARAQAGAMLLFGLCWGPYVATLLLSVLAYEQRPPLGPGTLLSLLSLGSASAAAVPVAMGLGDQRYTAPWRAAAQRCLQGLWGRASRDSPGPSIAYHPSSQSSVDLDLN

SEQ ID NO: 3 - DNA, 31 bp, Artificial
  Primer Sequence
gacaagcatgacgcccaacagcactggcgag

SEQ ID NO: 4 - DNA, 32 bp, Artificial
  Primer Sequence
cttgaattagttcaagtccaggtcgacactgc

SEQ ID NO: 5 - DNA, 1176 bp, Artificial
  Novel Sequence
atgtacccatacgacgtcccagactacgctggaagcttgacgcccaacagcactggcgaggtgcccagccccattcccaagggggctttggggctctccctggccctggcaagcctcatcatcaccgcgaacctgctcctagccctgggcatcgcctgggaccgccgcctgcgcagcccacctgctggctgcttcttcctgagcctactgctggctgggctgctcacgggtctggcattgcccacattgccagggctgtggaaccagagtcgccggggttactggtcctgcctcctcgtctacttggctcccaacttctccttcctctccctgcttgccaacctcttgctggtgcacggggagcgctacatggcagtcctgaggccactccagccccctgggagcattcggctggccctgctcctcacctgggctggtcccctgctctttgccagtctgcccgctctggggtggaaccactggacccctggtgccaactgcagctcccaggctatcttcccagccccctacctgtacctcgaagtctatgggctcctgctgcccgccgtgggtgctgctgccttcctctctgtccgcgtgctggccactgcccaccgccagctgcaggacatctgccggctggagcgggcagtgtgccgcgatgagccctccgccctggcccgggcccttacctggaggcaggcaagggcacaggctggagccatgctgctcttcgggctgtgctgggggccctacgtggccacactgctcctctcagtcctggcctatgagcagcgcccgccactggggcctgggacactgttgtccctcctctccctaggaagtgccagtgcagcggcagtgcccgtagccatggggctgggcgatcagcgctacacagccccctggagggcagccgcccaaaggtgcctgcaggggctgtggggaagagcctcccgggacagtcccggccccagcattgcctaccacccaagcagccaaagcagtgtcgacctggacttgaacgaattcggatccaagggcaattctgcagatatccagcacagtggcggccgctcgagtctagagggcccgcggttcgaaggtaagcctatccctaaccctctcctcggtctcgattctacgcgtaccggtcatcatcaccatcaccattga

SEQ ID NO: 6 - protein, 391 aa, Artificial [sequence omitted]

SEQ ID NO: 7 - DNA, 31 bp, Artificial
  Primer Sequence
gacaagcttgacgcccaacagcactggcgag

SEQ ID NO: 8 - DNA, 32 bp, Artificial
  Primer Sequence
cttgaattcgttcaagtccaggtcgacactgc

SEQ ID NO: 9 - DNA, 36 bp, Artificial
  Primer Sequence
gatcaagcttccatggcgtgctgcctgagcgaggag

SEQ ID NO: 10 - DNA, 53 bp, Artificial
  Primer Sequence
gatcggatccttagaacaggccgcagtccttcaggttcagctgcaggatggtg

SEQ ID NO: 11 - DNA, 21 bp, Artificial
  Primer Sequence
ctacctgtacctcgaagtcta

SEQ ID NO: 12 - DNA, 19 bp, Artificial
  Primer Sequence
agtggcgggcgctgctcat

SEQ ID NO: 13 - DNA, 21 bp, Artificial
  Mouse Primer Sequence
tgagctgtcggccattcccat

SEQ ID NO: 14 - DNA, 21 bp, Artificial
  Mouse Primer Sequence
gattgtccctcttggctcttc

(19) United States
(12) Patent Application Publication
(10) Pub. No.: US 2007/0231263 A1
(43) Pub. Date: Oct. 4, 2007

(54) Human G protein-coupled receptor and modulators thereof for the treatment of hyperglycemia and related disorders

(76) Inventors: Jun Qiu, San Diego, CA (US); Robert R. Webb, San Diego, CA (US); David J. Unett, San Deigo, CA (US); Joel E. Gatlin, San Diego, CA (US); Daniel T. Connolly, Solana Beach, CA (US)

Correspondence Address: BOZICEVIC, FIELD & FRANCIS LLP;(ARENA PHARMACEUTICALS, INC.), 1900 UNIVERSITY AVENUE, SUITE 200, EAST PALO ALTO, CA, 94303, US

(21) Appl. No.: 11/604,178

(22) Filed: Nov. 22, 2006

Related U.S. Application Data
(63) Continuation of application No. 11/578,257.
(60) Provisional application No. 60/561,954, filed on Apr. 13, 2004.

(51) Int. Cl. A61K 48/00; A61K 31/44; A61K 49/00; C07D 417/00; C07K 14/00; C12N 5/06

(52) U.S. Cl. 424/9.1; 435/366; 514/2; 514/336; 530/350; 546/268.7

(57) ABSTRACT

The present invention relates to methods of identifying whether one or more candidate compounds is a modulator of a G protein-coupled receptor (GPCR) or a modulator of blood glucose concentration. In certain embodiments, the GPCR is human. The present invention also relates to methods of using a modulator of the GPCR. A preferred modulator is agonist. Agonists of the invention are useful as therapeutic agents for lowering blood glucose concentration, for preventing or treating certain metabolic disorders, such as insulin resistance, impaired glucose tolerance, and diabetes, and for preventing or treating a complication of an elevated blood glucose concentration, such as atherosclerosis, heart disease, stroke, hypertension and peripheral vascular disease.

This application claims the benefit of priority from the following provisional application, filed via U.S. Express Mail with the United States Patent and Trademark Office on the indicated date: U.S. Provisional No. 601561,954, filed Apr. 13, 2004. The disclosure of the foregoing provisional application is herein incorporated by reference in its entirety.

### FIELD OF THE INVENTION

The present invention relates to methods of identifying whether one or more candidate compounds is a modulator of a G protein-coupled receptor (GPCR) or a modulator of blood glucose concentration. In certain embodiments, the GPCR is human. The present invention also relates to methods of using a modulator of the GPCR. A preferred modulator is agonist. Agonists of the invention are useful as therapeutic agents for lowering blood glucose concentration, for preventing or treating certain metabolic disorders, such as insulin resistance, impaired glucose tolerance, and diabetes, and for preventing or treating a complication of an elevated blood glucose concentration, such as atherosclerosis, heart disease, stroke, hypertension and peripheral vascular disease.

### BACKGROUND OF THE INVENTION

The following discussion is intended to facilitate the understanding of the invention, but is not intended nor admitted to be prior art to the invention.

A. Hyperglycemia

Blood glucose concentration typically is maintained within a narrow range. An elevation in blood glucose concentration normally leads to an increased release of insulin, which then acts on target cells to increase glucose uptake. Dysregulation of blood glucose homeostasis can lead to persistent elevated blood glucose concentration, or hyperglycemia. Some individuals with hyperglycemia may proceed to develop type 2 diabetes. Chronic exposure of tissues to hyperglycemia may result in diverse complications including microvascular problems of neuropathy, retinopathy and nephropathy and the macrovascular complications of stroke, coronary heart disease, and peripheral vascular disease. Hyperglycemia is a major and growing medical problem in need of better management options [Nesto, Reviews in Cardiovascular Medicine (2003)4:S11-S18; the disclosure of which is hereby incorporated by reference in its entirety].

B. G Protein-Coupled Receptors

Although a number of receptor classes exist in humans, by far the most abundant and therapeutically relevant is represented by the G protein-coupled receptor (GPCR) class It is estimated that there are some 30,00040,000 genes within the human genome, and of these, approximately 2% are estimated to code for GPCRs.

GPCRs represent an important area for the development of pharmaceutical products: from approximately 20 of the 100 known GPCRs, approximately 60% of all prescription pharmaceuticals have been developed. For example, in 1999, of the top 100 brand name prescription drugs, the following drugs interact with GPCRs (the primary diseases and/or disorders treated related to the drug is indicated in parentheses):
Claritin® (allergies) Prozac® (depression) Vasotoc® (hypertension)
Paxil® (depression) Zoloft® (depression) Zyprexa®(psychotic disorder)
Cozaar® (hypertension) I mitrex® (migraine) Zantac® (reflux)
Propulsid® (reflux disease) Risperdal® (schizophrenia) Serevent® (asthma)
Pepcid® (reflux) Gaster® (ulcers) Atrovent® (bronchospasm)
Effexor® (depression) Depakote® (epilepsy) Cardura® (prostatic hypertrophy)
Allegra® (allergies) Lupron® (prostate cancer) Zoladex® (prostate cancer)
Diprivan® (anesthesia) BuSpar® (anxiety) Ventolin® (bronchospasm)
Hytrin® (hypertension) Wellbutrin® (depression) Zyrtec® (rhinitis)
Plavix® (MI/stroke) Toprol-XL® (hypertension) Tenormin® (angina)
Xalatan® (glaucoma) Singulair® (asthma) Diovan® (hypertension)
Harnal® (prostatic hyperplasia)
(Med Ad News 1999 Data).

GPCRs share a common structural motif, having seven sequences of between 22 to 24 hydrophobic amino acids that form seven alpha helices, each of which spans the membrane (each span is identified by number, i.e., transmembrane-1 (TM-1), transmembrane-2 (TM-2), etc.). The transmembrane helices are joined by strands of amino acids between transmembrane-2 and transmembrane-3, transmembrane-4 and transmembrane-5, and transmembrane6 and transmembrane-7 on the exterior, or “extracellular” side, of the cell membrane (these are referred to as “extracellular” regions 1, 2 and 3 (EC-1, EC-2 and EC-3), respectively). The transmembrane helices are also joined by strands of amino acids between transmembrane-1 and transmembrane-2, transmembrane-3 and transmembrane-4, and transmembrane-5 and transmembrane6 on the interior, or “intracellular” side, of the cell membrane (these are referred to as “intracellular regions 1, 2 and 3 (IC-1, IC-2 and IC-3), respectively). The “carboxy” (“C”) terminus of the receptor lies in the intracellular space within the cell, and the “amino” (“N”) terminus of the receptor lies in the extracellular space outside of the cell.

Generally, when a ligand binds with the receptor (often referred to as “activation” of the receptor), there is a change in the conformation of the receptor that facilitates coupling between the intracellular region and an intracellular “G-protein.” It has been reported that GPCRs are “promiscuous” with respect to G proteins, i.e., that a GPCR can interact with more than one G protein. See, Kenakin, T., 43 Life Sciences 1095 (1988). Although other G proteins exist, currently, Gq, Gs, Gi, Gz and Go are G proteins that have been identified. Ligand-activated GPCR coupling with the G-protein initiates a signaling cascade process (referred to as “signal transduction”). Under normal conditions, signal transduction ultimately results in cellular activation or cellular inhibition. Although not wishing to be bound to theory, it is thought that the IC-3 loop as well as the carboxy terminus of the receptor interact with the G protein.

There are also promiscuous G proteins, which appear to couple several classes of GPCRs to the phospholipase C pathway, such as Gα15 or Gα16 [Offermanns & Simon, J Biol Chem (1995) 270:15175-80], or chimeric G proteins designed to couple a large number of different GPCRs to the same pathway, e.g. phospholipase C [Milligan & Rees, Trends in Pharmaceutical Sciences (1999) 20:118-24].

Under physiological conditions, GPCRs exist in the cell membrane in equilibrium between two different conformations: an inactive” state and an “active” state. A receptor in an inactive state is unable to link to the intracellular signaling transduction pathway to initiate signal transduction leading to a biological response. Changing the receptor conformation to the active state allows linkage to the transduction pathway (via the G-protein) and produces a biological response.

A receptor may be stabilized in an active state by a ligand or a compound such as a drug. Recent discoveries, including but not exclusively limited to modifications to the amino acid sequence of the receptor, provide means other than ligands or drugs to promote and stabilize the receptor in the active state conformation. These means effectively stabilize the receptor in an active state by simulating the effect of a ligand binding to the receptor. Stabilization by such ligand-independent means is termed “constitutive receptor activation.”

RUP43

RUP43 (where it is understood that endogenous RUP43 may be GPR131, e.g. GenBank® Accession No. NM_{—}170699) has recently been reported to act as a receptor for bile acid [European Patent Application Number 027171149 published as EP1378749 on 07 Jan. 2004; and Kawamata et al., J Biol Chem (2003) 278:9435-9440; the disclosure of each of which is hereby incorporated by reference in its entirety]. RUP43 expression within leukocytes was reported to be specific to monocytes, and bile acid acting at monocyte RUP43 was reported to inhibit expression of tumor necrosis factor alpha (TNFα). Compounds disclosed in EP1378749 may be used in methods of the subject invention.

### SUMMARY OF THE INVENTION

Applicants have unexpectedly discovered that agonists of RUP43 increase glucose uptake in adipocytes and in skeletal muscle cells. Applicants disclose that agonists of RUP43 have unexpected utility for lowering blood glucose concentration in a mammal. Applicants further disclose novel compounds having agonist activity at RUP43 and uses therefor.

In a first aspect, the invention features a method of identifying one or more candidate compounds as a modulator of a RUP43 GPCR, said receptor comprising a GPR131 amino acid sequence, wherein the receptor couples to a G protein; comprising the steps of:

(a) contacting the candidate compound with the, receptor, and

(b) determining whether the receptor functionality is modulated;

wherein a change in receptor functionality is indicative of the candidate compound being a modulator of a RUP43 GPCR.

In certain embodiments, the GPR131 amino acid sequence is selected from the group consisting of:
(a) the amino acid sequence of SEQ ID NO:2;
(b) amino acids 2-330 of SEQ ID NO:2;

(c) amino acids 2-330 of SEQ ID NO:2, with the proviso that the RUP43 G protein-coupled receptor does not comprise the methionine residue at amino acid position 1 of SEQ ID NO:2;

(d) the amino acid sequence of a G protein-coupled receptor encoded by a polynucleotide comprising a nucleic acid sequence, said nucleic acid sequence being obtainable by a process comprising performing PCR on a human DNA sample using primers SEQ ID NO:3 and SEQ ID NO:4;

(e) the amino acid sequence of SEQ ID NO:6;

(f) the amino acid sequence of a G protein-coupled receptor encoded by a polynucleotide comprising a nucleic acid sequence, said nucleic acid sequence being obtainable by a process comprising performing PCR on a human DNA sample using primers SEQ ID NO:7 and SEQ ID NO:8;

(g) the amino acid sequence of SEQ ID NO:2 wherein the alanine at amino acid position 223 of SEQ ID NO:2 is substituted with lysine;

(h) amino acids 2-330 of SEQ ID NO:2 wherein the alanine at amino acid position 223 of SEQ ID NO:2 is substituted with lysine;

(i) amino acids 2-330 of SEQ ID NO:2 wherein the alanine at amino acid position 223 of SEQ ID NO:2 is substituted with lysine, with the proviso that the RUP43 G protein-coupled receptor does not comprise the methionine residue at amino acid position 1 of SEQ ID NO:2; and

(j) the amino acid sequence of a G protein-coupled receptor encoded by a polynucleotide that hybridizes under stringent conditions to the complement of SEQ ID NO:1.

In certain embodiments, said RUP43 GPCR is recombinant. In certain embodiments, said contacting comprises contacting with a host cell or with membrane of a host cell that expresses the GPCR, wherein said host cell comprises an expression vector comprising a polynucleotide encoding the receptor.

In some embodiments, said contacting is carried out in the presence of a known ligand of the GPCR. In some embodiments, said contacting is cared out in the presence of a known modulator of the GPCR. In some embodiments, said contacting is carried out in the presence of a known agonist of the GPCR. In some embodiments, said known agonist of the GPCR is Compound 1, Compound 2, or Compound 3. In some embodiments, said known agonist of the GPCR is Compound 1. In some embodiments, said known agonist of the GPCR is Compound 2. In some embodiments, said known agonist of the GPCR is Compound 3. In some embodiments, said known agonist is present at about EC50 to about EC75 for the means of said determining.

The invention also relates to a method of identifying one or more candidate compounds as a modulator of blood glucose concentration in a mammal, comprising the steps of:

contacting the candidate compound with a GPCR comprising a GPR131 amino acid sequence, wherein the receptor couples to a G protein; and

determining whether the receptor functionality is modulated;

wherein a change in receptor functionality is indicative of the candidate compound being a modulator of blood glucose concentration in a mammal.

In certain embodiments, the GPR131 amino acid sequence is selected from the group consisting of
(a) the amino acid sequence of SEQ ID NO:2;
(b) amino acids 2-330 of SEQ ID NO:2;

(c) amino acids 2-330 of SEQ ID NO:2, with the proviso that the RUP43 G protein-coupled receptor does not comprise the methionine residue at amino acid position 1 of SEQ ID NO:2;

(d) the amino acid sequence of a G protein-coupled receptor encoded by a polynucleotide comprising a nucleic acid sequence, said nucleic acid sequence being obtainable by a process comprising performing PCR on a human DNA sample using primers SEQ ID NO:3 and SEQ ID NO:4;

(e) the amino acid sequence of SEQ ID NO:6;

(f) the amino acid sequence of a G protein-coupled receptor encoded by a polynucleotide comprising a nucleic acid sequence, said nucleic acid sequence being obtainable by a process comprising performing PCR on a human DNA sample using primers SEQ ID NO:7 and SEQ ID NO:8;

(g) the amino acid sequence of SEQ ID NO:2 wherein the alanine at amino acid position 223 of SEQ ID NO:2 is substituted with lysine;

(h) amino acids 2-330 of SEQ ID NO:2 wherein the alanine at amino acid position 223 of SEQ ID NO:2 is substituted with lysine;

(i) amino acids 2-330 of SEQ ID NO:2 wherein the alanine at amino acid position 223 of SEQ ID NO:2 is substituted with lysine, with the proviso that the RUP43 G protein-coupled receptor does not comprise the methionine residue at amino acid position 1 of SEQ ID NO:2; and

(j) the amino acid sequence of a G protein-coupled receptor encoded by a polynucleotide that hybridizes under stringent conditions to the complement of SEQ ID NO:1.

In certain embodiments, an increase in receptor functionality is indicative of the candidate compound being a compound that lowers blood glucose concentration in a mammal.

In certain embodiments, said GPCR is recombinant. In certain embodiments, said contacting comprises contacting with a host cell or with membrane of a host cell that expresses the GPCR, wherein said host cell comprises an expression vector comprising a polynucleotide encoding the receptor.

In some embodiments, said contacting is carried out in the presence of a known ligand of the GPCR. In some embodiments, said contacting is carried out in the presence of a known modulator of the GPCR. In some embodiments, said contacting is carried out in the presence of a known agonist of the GPCR. In some embodiments, said known agonist of the GPCR is Compound 1, Compound 2, or Compound 3. In some embodiments, said known agonist of the GPCR is Compound 1. In some embodiments, said known agonist of the GPCR is Compound 2. In some embodiments, said known agonist of the GPCR is Compound 3. In some embodiments, said known agonist is present at about EC50 to about EC75 for the means of said determining.

In certain embodiments, said one or more candidate compounds is not an antibody or an antigen-binding derivative thereof.

In certain embodiments, said one or more candidate compounds is not a peptide.

In certain embodiments, said one or more candidate compounds is not a bile acid.

In some embodiments, the GPR131 amino acid sequence is the amino acid sequence of SEQ ID NO:2. In some embodiments, the GPR131 amino acid sequence is a variant of the amino acid sequence of SEQ ID NO:2. In some embodiments, said variant of the amino acid sequence of SEQ ID NO:2 is an allelic variant or mammalian ortholog of said amino acid sequence. In some embodiments, said variant of the amino acid sequence of SEQ ID NO:2 is a non-endogenous, constitutively activated mutant of said amino acid sequence or of an allelic variant or mammalian ortholog of said amino acid sequence. In certain embodiments, said variant of the amino acid sequence of SEQ ID NO:2 is a biologically active fragment of said amino acid sequence or of an allelic variant or mammalian ortholog of said amino acid sequence. In certain embodiments, said biologically active fragment of the amino acid sequence of SEQ 11D NO:2 or of an allelic variant or mammalian ortholog of said amino acid sequence is the amino acid sequence of SEQ ID NO:2 or of an allelic variant or mammalian ortholog of said amino acid sequence absent the N-terminal methionine. In certain embodiments, said variant of the amino acid sequence of SEQ ID NO:2 is at least about 75%, at least about 80%, at least about 85%, at least about 90%, at least about 91%, at least about 92%, at least about 93%, at least about 94%, at least about 95%, at least about 96%, at least about 97% at least about 98% or at least about 99% identical to the amino acid sequence of SEQ ID NO:2. In some embodiments, said variant of the amino acid sequence of SEQ ID NO:2 is at least about 90%, at least about 91%, at least about 92%, at least about 93%, at least about 94%, at least about 95%, at least about 96% least about 97%, at least about 98% or at least about 99% identical to the amino acid sequence of SEQ ID NO:2.

In certain embodiments, said RUP43 GPCR comprising a GPR131 amino acid sequence is a fusion protein further comprising one or more epitope tags. In some embodiments, said fusion protein comprising one or more epitope tags is the amino acid sequence of SEQ ID NO:6.

In certain embodiments, said G protein leads to an increase in the level of intracellular cAMP. In some preferred embodiments, said G protein is Gs.

In certain embodiments, said G protein is pertussis toxin sensitive. In certain embodiments, said G protein is Gi or Go. In certain embodiments, said G protein is Gl. In certain embodiments, said G protein is Go.

In certain embodiments, said G protein is Gα15 or Gα16. In certain embodiments, said G protein is Gα15. In certain embodiments, said G protein is Gα16.

In certain embodiments, said G protein is Gq.

In certain embodiments, said method further comprises the step of comparing the modulation of the receptor caused by the candidate compound to a second modulation of the receptor caused by contacting the receptor with a known modulator of the receptor. In cerain embodiments, said known modulator is an agonist. In certain embodiments, said agonist is Compound 1, Compound 2, or Compound 3. In certain embodiments, said agonist is Compound 1. In certain embodiments, said agonist is Compound 2. In certain embodiments, said agonist is Compound 3.

In some preferred embodiments, said determining or said comparing is through the measurement of GTPγS binding to membrane comprising said GPCR. In certain embodiments, said GTPγS is labeled with [³⁵S].

In certain embodiments, said determining or said comparing is through the measurement of the level of a second messenger selected from the group consisting of cyclic AMP (cAMP), cyclic GMP (cGMP), inositol triphosphate (IP₃), diacylglycerol (DAG), MAP kinase activity, and Ca²⁺. In certain preferred embodiments, said second messenger is cAMP. In certain preferred embodiments, the level of cAMP is increased. In certain embodiments, said measurement of cAMP is carried out using whole-cell adenylyl cyclase assay. In certain embodiments, said measurement of cAMP is carried out with membrane comprising said GPCR. In certain embodiments, said second messenger is MAP kinase activity. In certain embodiments, the level of MAP kinase activity is increased.

In some preferred embodiments, said determining or said comparing is through CRE-reporter assay. In certain embodiments, said reporter is luciferase. In some embodiments, said reporter is β-galactosidase.

In certain embodiments, said determining or said comparing is through measurement of intracellular IP₃.

In certain embodiments, said determining or said comparing is through measurement of intracellular Ca²⁺.

In certain embodiments, said determining or said comparing is through measurement of glucose uptake by adipocytes obtained from a mammal.

In certain embodiments, said determining or said comparing is through measurement of glucose uptake by skeletal muscle cells obtained from a mammal.

In certain preferred embodiments, said determining or said comparing is through the use of a Melanophore assay.

In a second aspect, the invention features a compound of Formula (II):

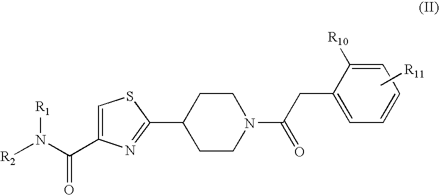

or a pharmaceutically acceptable salt thereof,

wherein:

R₁ is H or C₁₋₆alkyl;

R₂ is a 2-methyl-4,5,6,7-tetrahydro-2H-indazol-3-yl group; or

R₁ and R₂ together with the nitrogen to which they are bonded form a 3,4-dihydro-2H-quinoline-1-yl group; and

R₁₀ and R₁₁ are each independently H or halogen

In a third aspect, the invention features a modulator of a GPCR identified according to a method of the first aspect. In certain embodiments, the modulator is not an antibody or an antigen-binding derivative thereof. In certain embodiments, the modulator is not a peptide. In certain embodiments, the modulator is not a bile acid. In certain embodiments, the modulator is a compound that increases glucose uptake in adipocytes obtained from a mammal. In certain embodiments, the modulator is a compound that increases glucose uptake in skeletal muscle cells obtained from a mammal

The invention also features a modulator of a GPCR identifiable according to a method of the first aspect. In certain embodiments, the modulator is not an antibody or an antigen-binding derivative thereof. In certain embodiments, the modulator is not a peptide. In certain embodiments, the modulator is a compound that increases glucose uptake in adipocytes obtained from a mammal. In certain embodiments, the modulator is a compound that increases glucose uptake in skeletal muscle cells obtained from a mammal.

In certain embodiments, said modulator is selected from the group consisting of agonist, partial agonist, inverse agonist and antagonist. In certain embodiments, said modulator is an agonist. In certain embodiments, said modulator is a partial agonist. In certain embodiments, said modulator is an inverse agonist. In certain embodiments, said modulator is an antagonist.

In certain embodiments, said modulator is preferably an agonist. In certain embodiments, said agonist is a compound according to the second aspect.

In some embodiments, said modulator is an agonist with an EC₅₀ of less than 10 μM, of less than 1 M, of less than 100 nM, or of less than 10 nM. In some embodiments, said modulator is an agonist with an EC₅₀ of less than a value selected from the interval of 10 nM to 10 μM. In some embodiments, said modulator is an agonist with an EC₅₀ of less than a value selected from the interval of 10 nM to 1 μM. In some embodiments, said modulator is an agonist with an EC₅₀ of less than a value selected from the interval of 10 nM to 100 nM. In certain embodiments, said EC50 is determined using an assay selected from the group consisting of: whole cell cAMP assay carried using transfected HEK293 cells expressing recombinant RUP43 GPCR polypeptide having the amino acid sequence of SEQ ID NO:2 or 6; and melanophore assay carried out using transfected melanophores expressing recombinant RUP43 GPCR polypeptide having the amino acid sequence of SEQ ID NO:2 or 6. In some embodiments, said modulator is an agonist with an EC₅₀ of less than 10 μM, of less than 1 μM, of less than 100 nM, or of less than 10 nM in said assay. In some embodiments, said modulator is an agonist with an EC₅₀ of less than 10 μM in said assay, of less than 9 μM in said assay, of less than 8 μM in said assay, of less than 7 μM in said assay, of less than 6 μM in said assay, of less than 5 μM in said assay, of less than 4 μM in said assay, of less than 3 μM in said assay, of less than 2 μM in said assay, of less than 1 μM in said assay, of less than 900 nM in said assay, of less than 800 nM in said assay, of less than 700 nM in said assay, of less than 600 nM in said assay, of less than 500 nM in said assay, of less than 400 nM in said assay, of less than 300 nM in said assay, of less than 200 nM in said assay, of less than 100 nM in said assay, of less than 90 nM in said assay, of less than 80 nM in said assay, of less than 70 nM in said assay, of less than 60 nM in said assay, of less than 50 nM in said assay, of less than 40 nM n said assay, of less than 30 nM in said assay, of less than 20 nM in said assay, or of less than 10 nM in said assay. In some embodiments, said modulator is an agonist with an EC₅₀ in said assay of less than a value selected from the interval of 10 nM to 10 μM. In some embodiments, said modulator is an agonist with an EC₅₀ in said assay of less than a value selected from the interval of 10 nM to 1 μM. In some embodiments, said modulator is an agonist with an EC₅₀ in said assay of less than a value selected from the interval of 10 nM to 100 nM.

In some embodiments, said modulator is selective for the GPCR.

In some embodiments, said modulator is Compound 1 (“Cmpd#1, see Table 1), Compound 2 (“Cmpd#2”, see Table 1), or Compound 3 (“Cmpd#3”, see Table 1). In some embodiments, said modulator is Compound 1. In some embodiments, said modulator is Compound 2. In some embodiments, said modulator is Compound 3.

In some embodiments, said modulator is orally bioavailable. In some embodiments, said oral bioavailability is at least 1%, at least 5%, at least 10%, at least 15%, at least 20%, at least 25%, at least 30% at least 35%, at least 40%, or at least 45% relative to intraperitoneal administration. In some embodiments, said oral bioavailablity is at least 20%, at least 25%, at least 30%, at least 35%, at least 40%, or at least 45% relative to intraperitoneal administration.

In some embodiments, said orally bioavailable modulator is further able to cross the blood-brain barrier.

In a fourth aspect, the invention features a method of preparing a pharmaceutical or physiologically acceptable composition comprising admixing a carrier and a modulator of a RUP43 GPCR, said receptor comprising a GPR131 amino acid sequence. In certain embodiments, the modulator is not an antibody or an antigen-binding derivative thereof. In certain embodiments, the modulator is not a peptide. In certain embodiments, the modulator is a compound that increases glucose uptake in adipocytes obtained from a mammal. In certain embodiments, the modulator is a compound that increases glucose uptake in skeletal muscle cells obtained from a mammal. In certain embodiments, the modulator is selected from the group consisting of agonist, partial agonist inverse agonist, and antagonist. In certain embodiments, the modulator is an agonist. In certain embodiments, the modulator is a partial agonist. In certain embodiments, the modulator is an inverse agonist. In certain embodiments, the modulator is an antagonist. In certain embodiments, the modulator is preferably an agonist. In certain embodiments, said agonist is a compound according to the second aspect.

The invention also features a method of preparing a pharmaceutical or physiologically acceptable composition which comprises identifying a modulator of a RUP43 GPCR, wherein said receptor comprises a GPR131 amino acid sequence, and then admixing a carrier and the modulator, wherein the modulator is identifiable by a method according to a method of the first aspect. In certain embodiments, the modulator is identified according to a method of the first aspect. In certain embodiments, the modulator is not an antibody or an antigen-binding derivative thereof. In certain embodiments, the modulator is not a peptide. In certain embodiments, the modulator is preferably an agonist. In certain embodiments, the modulator is a compound that increases glucose uptake in adipocytes obtained from a mammal. In certain embodiments, the modulator is a compound that increases glucose uptake in skeletal muscle cells obtained from a mammal. In certain embodiments, the modulator is selected from the group consisting of agonist, partial agonist, inverse agonist, and antagonist. In certain embodiments, the modulator is an agonist. In certain embodiments, the modulator is a partial agonist. In certain embodiments, the modulator is an inverse agonist. In certain embodiments, the modulator is an antagonist. In certain embodiments, the modulator is preferably an agonist. In certain embodiments, said agonist is a compound according to the second aspect. In certain embodiments, said composition is pharmaceutical. In certain embodiments, said composition is physiologically acceptable.

In some embodiments, said modulator is an agonist with an EC₅₀ of less tan 10 μM of less than 1 μM, of less than 100 nM or of less than 10 nM. In some embodiments, said modulator is an agonist with an EC₅₀ of less than a value selected from the interval of 10 nM to 10 μM. In some embodiments, said modulator is an agonist with an EC₅₀ of less than a value selected from the interval of 10 nM to 1 μM. In some embodiments, said modulator is an agonist with an EC₅₀ of less than a value selected from the interval of 10 nM to 100 nM. In certain embodiments, said EC50 is determined using an assay selected from the group consisting of: whole cell cAMP assay carried using transfected HEK293 cells expressing recombinant RUP43 GPCR polypeptide having the amino acid sequence of SEQ ID NO:2 or 6; and melanophore assay carried out using transfected melanophores expressing recombinant RUP43 GPCR polypeptide having the amino acid sequence of SEQ ID NO:2 or 6. In some embodiments, said modulator is an agonist with an EC₅₀ of less than 10 μM of less than 1 μM, of less than 100 nM, or of less than 10 nM in said assay. In some embodiments, said modulator is an agonist with an EC₅₀ of less than 10 μM in said assay, of less than 9 μM in said assay, of less than 8 μM in said assay, of less than 7 μM in said assay, of less than 6 μM in said assay, of less than 5 μM in said assay, of less than 4 μM in said assay, of less than 3 μM in said assay, of less than 2 μM in said assay, of less than 1 μM in said assay, of less than 900 nM in said assay, of less than 800 nM in said assay, of less than 700 nM in said assay, of less than 600 nM in said assay, of less than 500 nM in said assay, of less than 400 nM in said assay, of less than 300 nM in said assay, of less than 200 nM in said assay, of less than 100 nM in said assay, of less than 90 nM in said assay, of less than 80 nM in said assay, of less than 70 nM in said assay, of less than 60 nM in said assay, of less than 50 nM in said assay, of less than 40 nM n said assay, of less than 30 nM in said assay, of less than 20 nM in said assay, or of less than 10 nM in said assay. In some embodiments, said modulator is an agonist with an EC₅₀ in said assay of less than a value selected from the interval of 10 nM to 10 μM. In some embodiments, said modulator is an agonist with an EC₅₀ in said assay of less than a value selected from the interval of 10 nM to 1 μM. In some embodiments, said modulator is an agonist with an EC₅₀ in said assay of less than a value selected from the interval of 10 nM to 100 nM.

In some embodiments, said modulator is selective for the GPCR.

In some embodiments, said modulator is Compound 1, Compound 2, or Compound 3. In some embodiments, said modulator is Compound 1. In some embodiments, said modulator is Compound 2. In some embodiments, said modulator is Compound 3.

In some embodiments, said modulator is orally bioavailable. In some embodiments, said oral bioavailability is at least 1%, at least 5%, at least 10%, at least 15%, at least 20%, at least 25%, at least 30%, at least 35%, at least 40%, or at least 45% relative to intraperitoneal admintration. In some embodiments, said oral bioavailablity is at least 20%, at least 25%, at least 30%, at least 35%, at least 40%, or at least 45% relative to intraperitoneal administration.

In some embodiments, said orally bioavailable modulator is further able to cross the blood-brain barrier.

In a fifth aspect, the invention features a method of modulating the activity of a RUP43 GPCR, said receptor comprising a GPR131 amino acid sequence, comprising the step of contacting the receptor with a modulator of the receptor. In certain embodiments, the modulator is identifiable by a method according to a method of the first aspect. In certain embodiments, the modulator is identified according to a method of the first aspect. In certain embodiments, the modulator is not an antibody or an antigen-binding derivative thereof. In certain embodiments, the modulator is not a peptide. In certain embodiments, the modulator is selected from the group consisting of agonist, partial agonist, inverse agonist, and antagonist. In certain embodiments, the modulator is an agonist. In certain embodiments, the modulator is a partial agonist. In certain embodiments, the modulator is an inverse agonist. In certain embodiments, the modulator is an antagonist. In certain embodiments, the modulator is preferably an agonist. In certain embodiments, the modulator is a compound that increases glucose uptake in adipocytes obtained from a mammal. In certain embodiments, the modulator is a compound that increases glucose uptake in skeletal muscle cells obtained from a mammal. In certain embodiments, said agonist is a compound according to the second aspect.

The invention also features a method of modulating the activity of a RUP43 GPCR, said receptor comprising a GPR131 amino acid sequence, comprising the step of contacting the receptor with a modulator of the receptor, wherein the modulator is identifiable by a method of the first aspect. In certain embodiments, the modulator is identified according to a method of the first aspect. In certain embodiments, the modulator is not an antibody or an antigen-binding derivative thereof. In certain embodiments, the modulator is not a peptide. In certain embodiments, the modulator is a compound that stimulates glucose uptake in adipocytes obtained from a mammal. In certain embodiments, the modulator is a compound that stimulates glucose uptake in skeletal muscle cells obtained from a mammal. In certain embodiments, the modulator is selected from the group consisting of agonist, partial agonist, inverse agonist, and antagonist. In certain embodiments, the modulator is an agonist. In certain embodiments, the modulator is a partial agonist. In certain embodiments, the modulator is an inverse agonist. In certain embodiments, the modulator is an antagonist. In certain embodiments, the modulator is preferably an agonist. In certain embodiments, said agonist is a compound according to the second aspect.

In some embodiments, said modulator is an agonist with an EC₅₀ of less than 10 μM, of less than 1 μM, of less than 100 nM, or of less than 10 nM. In some embodiments, said modulator is an agonist with an EC₅₀ of less than a value selected from the interval of 10 nM to 10 μM. In some embodiments, said modulator is an agonist with an EC₅₀ of less than a value selected from the interval of 10 nM to 1 μM. In some embodiments, said modulator is an agonist with an EC₅₀ of less than a value selected from the interval of 10 nM to 100 nM. In certain embodiments, said EC50 is determined using an assay selected from the group consisting of whole cell cAMP assay carried using transfected HEK293 cells expressing recombinant RUP43 GPCR polypeptide having the amino acid sequence of SEQ ID NO:2 or 6; and melanophore assay carried out using transfected melanophores expressing recombinant RUP43 GPCR polypeptide having the amino acid sequence of SEQ ID NO:2 or 6. In some embodiments, said modulator is an agonist with an EC₅₀ of less than 10 μM, of less than 1 μM, of less than100 nM, or of less than 10nM in said assay. In some embodiments, said modulator is an agonist with an EC₅₀ of less than 10 μM in said assay, of less than 9 μM in said assay, of less than 8 μM in said assay, of less than 7 μM in said assay, of less than 6 μM in said assay, of less than 5 μM in said assay, of less than 4 μM in said assay, of less than 3 μM in said assay, of less than 2 μM in said assay, of less than 1 μM in said assay, of less than 900 nM in said assay, of less than 800 nM in said assay, of less than 700 nM in said assay, of less than 600 nM in said assay, of less than 500 nM in said assay, of less than 400 nM in said assay, of less than 300 nM in said assay, of less than 200 nM in said assay, of less than 100 nM in said assay, of less than 90 nM in said assay, of less than 80 nM in said assay, of less than 70 nM in said assay, of less than 60 nM in said assay, of less than 50 nM in said assay, of less than 40 nM n said assay, of less than 30 nM in said assay, of less than 20 nM in said assay, or of less than 10 nM in said assay. In some embodiments, said modulator is an agonist with an EC₅₀ in said assay of less than a value selected from the interval of 10 nM to 10 μM. In some embodiments, said modulator is an agonist with an EC₅₀ in said assay of less than a value selected from the interval of 10 nM to 1 μM. In some embodiments, said modulator is an agonist with an EC₅₀ in said assay of less than a value selected from the interval of 10 nM to 100 nM.

In some embodiments, said modulator is selective for the GPCR.

In some embodiments, said modulator is Compound 1, Compound 2, or Compound 3. In some embodiments, said modulator is Compound 1. In some embodiments, said modulator is Compound 2. In some embodiments, said modulator is Compound 3.

In some embodiments, said modulator is orally bioavailable. In some embodiments, said oral bioavailability is at least 1%, at least 5%, at least 10%, at least 150/%, at least 20%, at least 25%, at least 30%, at least 35%, at least 40%, or at least 45% relative to intraperitoneal administration. In some embodiments, said oral bioavailablity is at least 20%, at least 25%, at least 30%, at least 35%, at least 40%, or at least 45% relative to intraperitoneal administration.

In some embodiments, said orally bioavailable modulator is further able to cross the blood-brain barrier.

In certain embodiments, said contacting comprises administration of the modulator to a membrane comprising the receptor.

In certain embodiments, said contacting comprises administration of the modulnor to a cell comprising the receptor.

In certain embodiments, said contacting comprises administration of the modtor to a tissue comprising the receptor.

In certain embodiments, said contacting comprises administration of the modulator to an individual comprising the receptor. In certain embodiments, said administration of the modulator to an individual comprising the receptor is oral. In certain embodiments, said individual is a mammal. In certain embodiments, said individual is a non-human mammal. In certain embodiments, said mammal is a horse, cow, sheep, pig, cat, dog, rabbit, mouse, rat, non-human primate or hank. In certain embodiments, said mammal is a mouse, rat, non-human primate, or human. Most preferred is human.

In a sixth aspect, the invention features a method of modulating the activity of a RUP43 GPCR, said receptor comprising a GPR131 amino acid sequence, wherein said modulation is for lowering blood glucose concentration in an individual in need of said modulation, comprising contacting said receptor with a therapeutically effective amount of a modulator of the receptor. In certain embodiments, the modulator is an agonist.

The invention also features a method of modulating the activity of a RUP43 GPCR, said receptor comprising a GPR131 amino acid sequence, wherein said modulation is for preventing or treating a metabolic disorder in an individual in need of said modulation, comprising contacting said receptor with a therapeutically effective amount of a modulator of the receptor. In certain embodiments, the modulator is an agonist. In certain embodiments, the metabolic disorder is selected from the group consisting of

(a) diabetes;

(b) impaired glucose tolerance;

(c) insulin resistance; and

(d) hyperinsulinemia.

In some embodiments, diabetes is type 1 diabetes. In certain preferred embodiments, diabetes is type 2 diabetes. In certain embodiments, the metabolic disorder is diabetes. In certain embodiments, the metabolic disorder is type 1 diabetes. In certain embodiments, the metabolic disorder is type 2 diabetes. In certain embodiments, the metabolic disorder is impaired glucose tolerance. In certain embodiments, the metabolic disorder is insulin resistance. In certain embodiments, the metabolic disorder is hyperinsulinemia. In certain embodiments, the metabolic disorder is related to an elevated blood glucose concentration in the individual.

The invention, also features a method of modulating the activity of a RUP43 GPCR, said receptor comprising a GPR131 amino acid sequence, wherein said modulation is for preventing or treating a complication of an elevated blood glucose concentration in an individual in need of said modulation, comprising contacting said receptor with a therapeutically effective amount of a modulator of the receptor. In certain embodiments, the modulator is an agonist. In certain embodiments, the complication is selected from the group consisting of:

(a) Syndrome X;

(b) atherosclerosis;

(c) atheromatous disease;

(d) heart disease;

(e) hypertension;

(f) stroke;

(g neuropathy,

(h) retinopathy,

(i) nephropathy, and

(j) peripheral vascular disease.

Heart disease includes, but is not limited to, cardiac insufficiency, coronary insufficiency, coronary artery disease, and high blood pressure. In certain embodiments, the complication is Syndrome X. In certain embodiments, the complication is atherosclerosis. In certain embodiments, the complication is atheromatous disease. In certain embodiments, the complication is heart disease. In certain embodiments, the complication is cardiac insufficiency. In certain embodiments, the complication is coronary insufficiency. In certain embodiments, the complication is coronary artery disease. In certain embodiments, the complication is coronary artery disease. In certain embodiments, the complication is high blood pressure. In certain embodiments, the complication is hypertension. In certain embodiments, the complication is stroke. In certain embodiments, the complication is neuropathy. In certain embodiments, the complication is retinopathy. In certain embodiments, the complication is neuropathy. In certain embodiments, the complication is peripheral vascular disease. In certain embodiments, the complication is polycystic ovary syndrome. In certain embodiments, the complication is hyperlipidemia.

In certain embodiments, the modulator is identifiable by a method according to a method of the first aspect. In certain embodiments, the modulator is identified according to a method of the first aspect. In certain embodiments, the modulator is not an antibody or an antigen-binding derivative thereof. In certain embodiments, the modulator is not a peptide. In certain embodiments, the modulator is a compound that stimulates glucose uptake in adipocytes obtained from a mammal. In certain embodiments, the modulator is a compound that stimulates glucose uptake in skeletal muscle cells obtained from a mammal. In certain embodiments, said modulator is selected from the group consisting of agonist, partial agonist, inverse agonist, and antagonist. In certain preferred embodiments, said modulator is an agonist. In certain embodiments, said agonist is a compound according to the second aspect.

In certain embodiments, said modulator is selective for the GPCR.

In some embodiments, said modulator is Compound 1, Compound 2, or Compound 3. In some embodiments, said modulator is Compound 1. In some embodiments, said modulator is Compound 2. In some embodiments, said modulator is Compound 3.

In certain embodiments, said modulator is orally bioavailable. In some embodiments, said oral bioavailability is at least 1%, at least 5%, at least 10%, at least 15%/, at least 20%, at least 25%, at least 30%, at least 35%, at least 40%, or at least 45% relative to intraperitoneal administration. In some embodiments, said oral bioavallablity is at least 20%, at least 25%, at least 300/., at least 35%, at least 40%, or at least 45% relative to intraperitoneal administration.

In certain embodiments, said orally bioavailable modulator is further able to cross the blood-brain barrier.

In some embodiments, said modulator is an agonist with an EC₅₀ of less than 10 μM of less than 1 μM of less than 100 nM, or of less than 10 nM. In some embodiments, said modulator is an agonist with an EC₅₀ of less than a value selected from the interval of 10 nM to 10 μM. In some embodiments, said modulator is an agonist with an EC₅₀ of less than a value selected from the interval of 10 nM to 1 μM. In some embodiments, said modulator is an agonist with an EC₅₀ of less than a value selected from the interval of 10 nM to 100 nM. In certain embodiments, said EC50 is determined using an assay selected from the group consisting of: whole cell cAMP assay carried using transfected HEK293 cells expressing recombinant RUP43 GPCR polypeptide having the amino acid sequence of SEQ ID NO:2 or 6; and melanophore assay carried out using transfected melanophores expressing recombinant RUP43 GPCR polypeptide having the amino acid sequence of SEQ ID NO:2 or 6. In some embodiments, said modulator is an agonist with an EC₅₀ of less than 10 μM, of less than 1 μM, of less than 100 nM, or of less than 10 nM in said assay. In some embodiments, said modulator is an agonist with an EC₅₀ of less than 10 μM in said assay, of less than 9 μM in said assay, of less than 8 μM in said assay, of less than 7 μM in said assay, of less than 6 μM in said assay, of less than 5 μM in said assay, of less than 4 μM in said assay, of less than 3 μM in said assay, of less than 2 μM in said assay, of less than 1 μM in said assay, of less than 900 nM in said assay, of less than 800 nM in said assay, of less than 700 nM in said assay, of less than 600 nM in said assay, of less man 500 nM in said assay, of less than 400 nM in said assay, of less than 300 nM in said assay, of less than 200 nM in said assay, of less than 100 nM in said assay, of less than 90 nM in said assay, of less than 80 nM in said assay, of less than 70 nM in said assay, of less than 60 nM in said assay, of less than 50nM in said assay, of less than 40 nM n said assay, of less than 30 nM in said assay, of less than 20 nM in said assay, or of less than 10 nM in said assay. In some embodiments, said modulator is an agonist with an EC₅₀ in said assay of less than a value selected from the interval of 10 nM to 10 μM. In some embodiments, said modulator is an agonist with an EC₅₀ in said assay of less than a value selected from the interval of 10 nM to 1 μM. In some embodiments, said modulator is an agonist with an EC₅₀ in said assay of less than a value selected from the interval of 10 nM to 100 nM.

In certain embodiments, said contacting comprises oral administration of said modulator to said individual.

In certain embodiments, said individual is a mammal. In certain embodiments, said individual is a non-human mammal. In certain embodiments, said mammal is a horse, cow, sheep, pig, cat, dog, rabbit, mouse, rat, non-human primate or human. In certain embodiments, said mammal is a mouse, rat, non-human primate, or human. Most preferred is human.

In a seventh aspect, the invention features a method of lowering blood glucose concentration in an individual in need of said lowering, comprising contacting a therapeutically effective amount of a modulator of a RUP43 GPCR with said receptor, said GPCR comprising a GPR131 amino acid sequence. In certain embodiments, the modulator is an agonist.

The invention additionally features a method of lowering blood glucose concentration in a mammal comprising providing or administering to a mammal in need of said lowering a modulator of RUP43 GPCR, said GPCR comprising a GPR131 amino acid sequence. In certain embodiments, the modulator is an agonist. In certain embodiments, the agonist of RUP43 GPCR is an agonist of GPR131 GPCR, where it is understood that GPR131 GPCR is endogenous RUP43 GPCR.

The invention also features a method of preventing or treating a metabolic disorder in an individual in need of said prevention or treatment, comprising contacting a therapeutically effective amount of a modulator of a RUP43 GPCR with said receptor, said receptor comprising a GPR131 amino acid sequence. In certain embodiments, the modulator is an agonist. In certain embodiments, the metabolic disorder is selected from the group consisting of:

(a) diabetes;

(b) impaired glucose tolerance;

(c) insulin resistance; and

(d) hyperinsulinemia.

The invention additionally features a method of preventing or treating a metabolic disorder comprising administering to a mammal in need of said prevention or treatment a modulator of RUP43 GPCR, said receptor comprising a GPR131 amino acid sequence. In certain embodiments, the modulator is an agonist. In certain embodiments, the agonist of RUP43 GPCR is an agonist of GPR131 GPCR, where it is understood that GPR131 GPCR is endogenous RUP43 GPCR. In certain embodiments, the metabolic disorder is selected from the group consisting of:

(a) diabetes;

(b) impaired glucose tolerance;

(c) insulin resistance; and

(d) hyperinsulinemia.

In some embodiments, diabetes is type 1 diabetes. In certain preferred embodiments, diabetes is type 2 diabetes. In certain embodiments, the metabolic disorder is diabetes. In certain embodiments, the metabolic disorder is type 1 diabetes. In certain embodiments, the metabolic disorder is type 2 diabetes. In certain embodiments, the metabolic disorder is impaired glucose tolerance. In certain embodiments, the metabolic disorder is insulin resistance. In certain embodiments, the metabolic disorder is hyperinsulinemia In certain embodiments, the metabolic disorder is related to an elevated blood glucose concentration in the individual.

The invention also features a method of preventing or treating a complication of an elevated blood glucose concentration in an individual in need of said prevention or treatment, comprising contacting a therapeutically effective amount of a modulator of a RUP43 GPCR with said receptor, said receptor comprising a GPR131 amino acid sequence. In certain embodiments, the modulator is an agonist. In certain embodiments, the complication is selected from the group consisting of

(a) Syndrome X;

(b) atherosclerosis;

(c) atheromatous disease;

(d) heart disease;

(e) hypertension;

(f) stroke;

(g) neuropathy;

(h) retinopathy;

(i) nephropathy; and

(j) peripheral vascular disease.

Heart disease includes, but is not limited to, cardiac insufficiency, coronary insufficiency, coronary artery disease, and high blood pressure. In certain embodiments, the complication is Syndrome X. In certain embodiments, the complication is atherosclerosis. In certain embodiments, the complication is atheromatous disease. In certain embodiments, the complication is heart disease. In certain embodiments, the complication is cardiac insufficiency. In certain embodiments, the complication is coronary insufficiency. In certain embodiments, the complication is coronary artery disease. In certain embodiments, the complication is high blood pressure. In certain embodiments, the complication is hypertension. In certain embodiments, the complication is stroke. In certain embodiments, the complication is neuropathy. In certain embodiments, the complication is retinopathy. In certain embodiments, the complication is neuropathy. In certain embodiments, the complication is peripheral vascular disease. In certain embodiments, the complication is polycystic ovary syndrome. In certain embodiments, the complication is hyperlipidemia.

The invention additionally features a method of preventing or treating a complication of an elevated blood glucose concentration comprising providing or administering to a mammal in need of said prevention or treatment a modulator of RUP43 GPCR, said receptor comprising a GPR131 amino acid sequence. In certain embodiments, the modulator is an agonist. In certain embodiments, the agonist of RUP43 GPCR is an agonist of GPR131 GPCR, where it is understood that GPR131 GPCR is endogenous RUP43 GPCR. In certain embodiments, the complication is selected from the group consisting of:

(a) Syndrome X;

(b) atherosclerosis;

(c) atheromatous disease;

(d) heart disease;

(e) hypertension;

(f) stroke;

(g) neuropathy;

(h) retinopathy;

(i) nephropathy, and

(j) peripheral vascular disease.

Heart disease includes, but is not limited to, cardiac insufficiency, coronary insufficiency, coronary artery disease, and high blood pressure. In certain embodiments, the complication is Syndrome X. In certain embodiments, the complication is atherosclerosis. In certain embodiments, the complication is atheromatous disease. In certain embodiments, the complication is heart disease. In certain embodiments, the complication is cardiac insufficiency. In certain embodiments, the complication is coronary insufficiency. In certain embodiments, the complication is coronary artery disease. In certain embodiments, the complication is high blood pressure. In certain embodiments, the complication is hypertension. In certain embodiments, the complication is stroke. In certain embodiments, the complication is neuropathy. In certain embodiments, the complication is retinopathy. In certain embodiments, the complication is neuropathy. In certain embodiments, the complication is peripheral vascular disease. In certain embodiments, the complication is polycystic ovary syndrome. In certain embodiments, the complication is hyperlipidemia.

In certain embodiments, the modulator is identifiable by a method according to a method of the first aspect. In certain embodiments, the modulator is identified according to a method of the first aspect. In certain embodiments, the modulator is not an antibody or an antigen-binding derivative thereof. In certain embodiments, the modulator is not a peptide. In certain embodiments, the modulator is a compound that stimulates glucose uptake in adipocytes obtained from a mammal. In certain embodiments, the modulator is a compound that stimulates glucose uptake in adipocytes obtained from the mammal. In certain embodiments, the modulator is a compound that stimulates glucose uptake in skeletal muscle cells obtained from a mammal. In certain embodiments, the modulator is a compound that stimulates glucose uptake in skeletal muscle cells obtained from the mammal. In certain embodiments, said modulator is selected from the group consisting of agonist, partial agonist, inverse agonist, and antagonist. In certain preferred embodiments, said modulator is an agonist. In certain embodiments, said agonist is a compound according to the second aspect

In certain embodiments, said modulator is selective for the GPCR.

In some embodiments, said modulator is Compound 1, Compound 2, or Compound 3. In some embodiments, said modulator is Compound 1. In some embodiments, said modulator is Compound 2. In some embodiments, said modulator is Compound 3.

In certain embodiments, said modulator is orally bioavailable. In some embodiments, said oral bioavailability is at least 1%, at least 5%, at least 10%, at least 15%, at least 20%, at least 25%, at least 30%, at least 35%, at least 40%, or at least 45% relative to intraperitoneal administration. In some embodiments, said oral bioavailablity is at least 20%, at least 25%, at least 30%, at least 35%, at least 40%, or at least 45% relative to intraperitoneal adminstration.

In certain embodiments, said orally bioavailable modulator is further able to cross the blood-brain barrier.

In some embodiments, said modulator is an agonist with an EC₅₀ of less than 10 μM, of less than 1 μM, of less than 100 nM, or of less than 10 nM. In some embodiments, said modulator is an agonist with an EC₅₀ of less than a value selected from the interval of 10 nM to 10 μM. In some embodiments, said modulator is an agonist with an EC₅₀ of less than a value selected from the in al of 10 nM to 1 μM. In some embodiments, said modulator is an agonist with an EC₅₀ of less than a value selected from the interval of 10 nM to 100 nM. In certain embodiments, said EC50 is determined using an assay selected from the group consisting of: whole cell cAMP assay carried using transfected HEK293 cells expressing recombinant RUP43 GPCR polypeptide having the amino acid sequence of SEQ ID NO:2 or 6; and melanophore assay carded out using transfected melanophores expressing recombinant RUP43 GPCR polypeptide having the amino acid sequence of SEQ ID NO:2 or 6. In some embodiments, said modulator is an agonist with an EC₅₀ of less than 10 μM, of less than 1 μM, of less than 100 nM or of less than 10 nM in said assay. In some embodiments, said modulator is an agonist with an EC₅₀ of less than 10 μM in said assay, of less than 9 μM in said assay, of less than 8 μM in said assay, of less than 7 μM in said assay, of less than 6 μM in said assay, of less than 5 μM in said assay, of less than 4 μM in said assay, of less than 3 μM in said assay, of less than 2 μM in said assay, of less than 1 μM in said assay, of less than 900 nM in said assay, of less than 800 nM in said assay, of less than 700 nM in said assay, of less than 600 nM in said assay, of less than500 nM in said assay, of less than 400 nM in said assay, of less than 300 nM in said assay, of less than 200 nM in said assay, of less than 100 nM in said assay, of less than 90 nM in said assay, of less than 80 nM in said assay, of less than 70 nM in said assay, of less than 60 nM in said assay, of less than 50 nM in said assay, of less than 40 nM n said assay, of less than 30 nM in said assay, of less than 20 nM in said assay, or of less than 10 nM in said assay. In some embodiments, said modulator is an agonist with an EC₅₀ in said assay of less than a value selected from the interval of 10 nM to 10 μM. In some embodiments, said modulator is an agonist with an EC50 in said assay of less than a value selected from the interval of 10 nM to 1 μM. In some embodiments, said modulator is an agonist with an EC₅₀ in said assay of less than a value selected from the interval of 10 nM to 100 nM.

In certain embodiments, said contacting comprises oral administration of said modulator to said individual.

In certain embodiments, said individual is a mammal. In certain embodiments, said individual is a non-human mammal. In certain embodiments, said mammal is a horse, cow, sheep, pig, cat, dog, rabbit, mouse, rat non-human primate or human. In certain embodiments, said mammal is a mouse, rat, non-human primate, or human. Most preferred is human.

In an eighth aspect, the invention features a pharmaceutical or physiologically acceptable composition comprising, consisting essentially of, or consisting of a modulator a RUP43 GPCR, said receptor comprising a GPR131 amino acid sequence.

In certain embodiments, the modulator is identifiable by a method according to a method of the first aspect. In certain embodiments, the modulator is identified according to a method of the first aspect. In certain embodiments, the modulator is not an antibody or an antigen-binding derivative thereof. In certain embodiments, the modulator is not a peptide. In certain embodiments, the modulator is a compound that stimulates glucose uptake in adipocytes obtained from a mammal. In certain embodiments, the modulator is a compound that stimulates glucose uptake in skeletal muscle cells obtained from a mammal. In certain embodiments, said modulator is selected from the group consisting of agonist, partial agonist, inverse agonist, and antagonist. In certain preferred embodiments, said modulator is an agonist. In certain embodiments, said agonist is a compound according to the second aspect.

In certain embodiments, said composition is pharmaceutical. In certain embodiments, the pharmaceutical composition comprises the modulator of a RUP43 GPCR. In certain embodiments, the pharmaceutical composition consists essentially of the modulator of a RUP43 GPCR. In certain embodiments, the pharmaceutical composition consists of the modulator of a RUP43 GPCR.

In certain embodiments, said composition is physiologically acceptable. In certain embodiments, the physiologically acceptable composition comprises the modulator of a RUP43 GPCR. In certain embodiments, the physiologically acceptable composition consists essentially of the modulator of a RUP43 GPCR. In certain embodiments, the physiologically acceptable composition consists of the modulator of a RUP43 GPCR.

In certain embodiments, said modulator is selective for the GPCR.

In some embodiments, said modulator is Compound 1, Compound 2, or Compound 3. In some embodiments, said modulator is Compound 1. In some embodiments, said modulator is Compound 2. In some embodiments, said modulator is Compound 3.

In certain embodiments, said modulator is orally bioavailable. In some embodiments, said oral bioavailability is at least 1%, at least 5%, at least 10%, at least 15%, at least 20%, at least 25%, at least 30%, at least 35%, at least 40%, or at least 45% relative to intraperitoneal administration. In some embodiments, said oral bioavailablity is at least 20%, at least 25%, at least 30%, at least 35%, at least 40%, or at least 45% relative to intraperitoneal administration.

In certain embodiments, said orally bioavailable modulator is further able to cross the blood-brain barrier.

In some embodiments, said modulator is an agonist with an EC₅₀ of less than 10 μM, of less than 1 μM, of less than 100 nM or of less than 10 nM. In some embodiments, said modulator is an agonist with an EC₅₀ of less than a value selected from the interval of 10 nM to 10 μM. In some embodiments, said modulator is an agonist with an EC₅₀ of less than a value selected from the interval of 10 nM to 1 μM. In some embodiments, said modulator is an agonist with an EC₅₀ of less than a value selected from the interval of 10 nM to 100 nM. In certain embodiments, said EC50 is determined using an assay selected from the group consisting of: whole cell cAMP assay carried using transfected HEK293 cells expressing recombinant RUP43 GPCR polypeptide having the amino acid sequence of SEQ ID NO:2 or 6; and melanophore assay carried out using transfected melanophores expressing recombinant RUP43 GPCR polypeptide having the amino acid sequence of SEQ ID NO:2 or 6. In some embodiments, said modulator is an agonist with an EC₅₀ of less than 10 μM, of less than 1 μM, of less than 100 nM, or of less than 10 nM in said assay. In some embodiments, said modulator is an agonist with an EC₅₀ of less than 10 μM in said assay, of less than 9 μM in said assay, of less than 8 μM in said assay, of less than 7 μM in said assay, of less than 6 μM in said assay, of less than 5 μM in said assay, of less than 4 μM in said assay, of less than 3 μM in said assay, of less than 2 μM in said assay, of less than 1 μM in said assay, of less than 900 nM in said assay, of less than 800 nM in said assay, of less than 700 nM in said assay, of less than 600 nM in said assay, of less than 500 nM in said assay, of less than 400 nM in said assay, of less than 300 nM in said assay, of less than 200 nM in said assay, of less than 100 nM in said assay, of less than 90 nM in said assay, of less than 80 nM in said assay, of less than 70 nM in said assay, of less than 60 nM in said assay, of less than 50 nM in said assay, of less than 40 nM n said assay, of less than 30 nM in said assay, of less than 20 nM in said assay, or of less than 10 nM in said assay. In some embodiments, said modulator is an agonist with an EC₅₀ in said assay of less than a value selected from the interval of 10 nM to 10 μM. In some embodiments, said modulator is an agonist with an EC₅₀ in said assay of less than a value selected from the interval of 10 nM to 1 μM. In some embodiments, said modulator is an agonist with an EC₅₀ in said assay of less than a value selected from the interval of 10 nM to 100 nM.

In a ninth aspect, the invention features a method of lowering blood glucose concentration comprising providing or administering to an individual in need of said lowering said pharmaceutical or physiologically acceptable composition of the eighth aspect.

The invention also features a method of preventing or treating a metabolic disorder comprising providing or administering to an individual in need of said prevention or treatment said pharmaceutical or physiologically acceptable composition of the eighth aspect. In certain embodiments, the metabolic disorder is selected from the group consisting of:

(a) diabetes;

(b). impaired glucose tolerance;

(c) insulin resistance; and

(d) hyperinsulinemia.

In some embodiments, diabetes is type 1 diabetes. In certain preferred embodiments, diabetes is type 2 diabetes. In certain embodiments, the metabolic disorder is diabetes. In certain embodiments, the metabolic disorder is type 1 diabetes. In certain embodiments, the metabolic disorder is type 2 diabetes. In certain embodiments, the metabolic disorder is impaired glucose tolerance. In certain embodiments, the metabolic disorder is insulin resistance. In certain embodiments, the metabolic disorder is hyperinsulinemia. In certain embodiments, the metabolic disorder is related to an elevated blood glucose concentration in the individual.

The invention also features a method of preventing or treating a complication of an elevated blood glucose concentration comprising providing or administering to an individual in need of said prevention or treatment said pharmaceutical or physiologically acceptable composition of the eighth aspect. In certain embodiments, the complication is selected from the group consisting of:

(a) Syndrome X;

(b) atherosclerosis;

(c) atheromatous disease;

(d) heart disease;

(e) hypertension;

(i) stroke;

(j) neuropathy;

(i) retinopathy;

(i) nephropathy; and

(j) peripheral vascular disease.

Heart disease includes, but is not limited to, cardiac insufficiency, coronary insufficiency, coronary artery disease, and high blood pressure. In certain embodiments, the complication is Syndrome X. In certain embodiments, the complication is atherosclerosis. In certain embodiments, the complication is atheromatous disease. In certain embodiments, the complication is heart disease. In certain embodiments, the complication is cardiac insufficiency. In certain embodiments, the complication is coronary insufficiency. In certain embodiments, the complication is coronary artery disease. In certain embodiments, the complication is high blood pressure. In certain embodiments, the complication is hypertension. In certain embodiments, the complication is stroke. In certain embodiments, the complication is neuropathy. In certain embodiments, the complication is retinopathy. In certain embodiments, the complication is neuropathy. In certain embodiments, the complication is peripheral vascular disease. In certain embodiments, the complication is polycystic ovary syndrome. In certain embodiments, the complication is hyperlipidemia.

In certain embodiments, said modulator is an agonist.

In certain embodiments, a therapeutically effective amount of said pharmaceutical or physiologically acceptable composition is provided or administered to said individual.

In certain embodiments, said providing or administering of said pharmaceutical or physiologically acceptable composition is oral.

In certain embodiments, said individual is a mammal. In certain embodiments, said individual is a non-human mammal. In certain embodiments, said mammal is a horse, cow, sheep, pig, cat, dog, rabbit, mouse, rat, non-human primate or human. In certain embodiments, said mammal is a mouse, rat, non-human primate, or human. Most preferred is human.

In a tenth aspect, the invention features a modulator of a RUP43 GPCR, said receptor comprising a GPR131 amino acid sequence, for use in a method of treatment of the human animal body by therapy.

In certain embodiments, the modulator is identifiable by a method according to a method of the first aspect. In certain embodiments, the modulator is identified according to a method of the first aspect. In certain embodiments, the modulator is not an antibody or an antigen-binding derivative thereof. In certain embodiments, the modulator is not a peptide. In certain embodiments, the modulator is a compound that stimulates glucose uptake in adipocytes obtained from a mammal. In certain embodiments, the modulator is a compound that stimulates glucose uptake in adipocytes obtained from the human or the animal. In certain embodiments, the modulator is a compound that stimulates glucose uptake in skeletal muscle cells obtained from a mammal. In certain embodiments, the modulator is a compound that stimulates glucose uptake in skeletal muscle cells obtained from the human or the animal. In certain embodiments, said modulator is selected from the group consisting of agonist, partial agonist, inverse agonist, and antagonist. In certain preferred embodiments, said modulator is an agonist. In certain embodiments, said agonist is a compound according to the second aspect.

In certain embodiments, said modulator is selective for the GPCR.

In some embodiments, said modulator is Compound 1, Compound 2, or Compound 3. In some embodiments, said modulator is Compound 1. In some embodiments, said modulator is Compound 2. In some embodiments, said modulator is Compound 3.

In certain embodiments, said modulator is orally bioavailable. In some embodiments, said oral bioavailability is at least 1%, at least 5%, at least 10%, at least 15%, at least 20%, at least 25%, at least 30%, at least 35%, at least 40%, or at least 45% relative to intraperitoneal administration. In some embodiments, said oral bioavallablity is at least 20%, at least 25%, at least 30%, at least 35%, at least 40%, or at least 45% relative to intraperitoneal administration.

In certain embodiments, said orally bioavailable modulator is further able to cross the blood-brain barrier.

In some embodiments, said modulator is an agonist with an EC50 of less than 10 μM, of less than 1 μM, of less than 100 nM, or of less than 10 nM. In some embodiments, said modulator is an agonist with an EC₅₀ of less than a value selected from the interval of 10 nM to 10 μM. In some embodiments, said modulator is an agonist with an EC₅₀ of less than a value selected from the interval of 10 nM to 1 μM. In some embodiments, said modulator is an agonist with an EC₅₀ of less than a value selected from the interval of 10 nM to 100 nM. In certain embodiments, said EC50 is determined using an assay selected from the group consisting of: whole cell cAMP assay carried using transfected HEK293 cells expressing recombinant RUP43 GPCR polypeptide having the amino acid sequence of SEQ ID NO:2 or 6; and melanophore assay carried out using transfected melanophores expressing recombinant RUP43 GPCR polypeptide having the amino acid sequence of SEQ ID NO:2 or 6. In some embodiments, said modulator is an agonist with an EC₅₀ of less than 10 μM, of less than 1 μM, of less than 100 nM, or of less than 10 nM in said assay. In some embodiments, said modulator is an agonist with an EC₅₀ of less than 10 μM in said assay, of less than 9 μM in said assay, of less than 8 μM in said assay, of less than 7 μM in said assay, of less than 6 μM in said assay, of less than 5 μM in said assay, of less than 4 μM in said assay, of less than 3 μM in said assay, of less than 2 μM in said assay, of less than 1 μM in said assay, of less than 900 nM in said assay, of less than 800 nM in said assay, of less than 700 nM in said assay, of less than 600 nM in said assay, of less than 500 nM in said assay, of less than 400 nM in said assay, of less than 300 nM in said assay, of less than 200 nM in said assay, of less tan 100 nM in said assay, of less than 90 nM in said assay, of less than 80 nM in said assay, of less than 70 nM in said assay, of less than 60 nM in said assay, of less than 50 nM in said assay, of less than 40 nM n said assay, of less than 30 nM in said assay, of less than 20 nM in said assay, or of less than 10 nM in said assay. In some embodiments, said modulator is an agonist with an EC₅₀ in said assay of less than a value selected from the interval of 10 nM to 10 μM. In some embodiments, said modulator is an agonist with an EC₅₀ in said assay of less than a value selected from the interval of 10 nM to 1 μM. In some embodiments, said modulator is an agonist with an EC₅₀ in said assay of less than a value selected from the interval of 10 nM to 100 nM.

In certain embodiments, said animal is a mammal. In certain embodiments, said mammal is a horse, cow, sheep, pig, cat, dog, rabbit, mouse, rat or non-human primate. More preferred of human or animal is human.

In an eleventh aspect, the invention features a modulator of a RUP43 GPCR, said receptor comprising a GPR131 amino acid sequence, for use in a method of lowering blood glucose concentration in the human animal body by therapy. In certain embodiments, the modulator is an agonist.

The invention also features a modulator of a RUP43 GPCR, said receptor comprising a GPR131 amino acid sequence, for use in a method of prevention of or treatment for a metabolic disorder in a human or animal body by therapy. In certain embodiments, the modulator is an agonist. In certain embodiments, the metabolic disorder is selected from the group consisting of:

(a) diabetes;

(b) impaired glucose tolerance;

(c) insulin resistance; and

(d) hyperinsulinemia.

In some embodiments, diabetes is type 1 diabetes. In certain preferred embodiments, diabetes is type 2 diabetes. In certain embodiments, the metabolic disorder is diabetes. In certain embodiments, the metabolic disorder is type 1 diabetes. In certain embodiments, the metabolic disorder is type 2 diabetes. In certain embodiments, the metabolic disorder is impaired glucose tolerance. In certain embodiments, the metabolic disorder is insulin resistance. In certain embodiments, the metabolic disorder is hyperinsulinemia. In certain embodiments, the metabolic disorder is related to an elevated blood glucose concentration in the individual.

The invention also features a modulator of a RUP43 GPCR, said receptor comprising a GPR131 amino acid sequence, for use in a method of prevention of or treatment for a complication of an elevated blood glucose concentration in a human or animal body by therapy. In certain embodiments, the modulator is an agonist. In certain embodiments, the complication is selected from the group consisting of:

(a) Syndrome X;

(b) atherosclerosis;

(c) atheromatous disease;

(d) heart disease;

(e) hypertension;

(f) stroke;

(g) neuropathy;

(h) retinopathy;

(i) nephropathy; and

(j) peripheral vascular disease.

Heart disease includes, but is not limited to, cardiac insufficiency, coronary insufficiency, coronary artery disease, and high blood pressure. In certain embodiments, the complication is Syndrome X. In certain embodiments, the complication is atherosclerosis. In certain embodiments, the complication is atheromatous disease. In certain embodiments, the complication is heart disease. In certain embodiments, the complication is cardiac insufficiency. In certain embodiments, the complication is coronary insufficiency. In certain embodiments, the complication is coronary artery disease. In certain embodiments, the complication is high blood pressure. In certain embodiments, the complication is hypertension. In certain embodiments, the complication is stroke. In certain embodiments, the complication is neuropathy. In certain embodiments, the complication is retinopathy. In certain embodiments, the complication is neuropathy. In certain embodiments, the complication is peripheral vascular disease. In certain embodiments, the complication is polycystic ovary syndrome. In certain embodiments, the complication is hyperlipidemia.

In certain embodiments, the modulator is identifiable by a method according to a method of the first aspect. In certain embodiments, the modulator is identified according to a method of the first aspect. In certain embodiments, the modulator is not an antibody or an antigen-binding derivative thereof. In certain embodiments, the modulator is not a peptide. In certain embodiments, the modulator is a compound that stimulates glucose uptake in adipocytes obtained from a mammal. In certain embodiments, the modulator is a compound that stimulates glucose uptake in adipocytes obtained from the human or animal. In certain embodiments, the modulator is a compound that stimulates glucose uptake in skeletal muscle cells obtained from a mammal. In certain embodiments, the modulator is a compound that stimulates glucose uptake in skeletal muscle cells obtained from the human or animal. In certain embodiments, said modulator is selected from the group consisting of agonist, partial agonist, inverse agonist and antagonist. In certain preferred embodiments, said modulator is an agonist. In certain embodiments, said agonist is a compound according to the second aspect.

In certain embodiments, said modulator is selective for the GPCR.

In some embodiments, said modulator is Compound 1, Compound 2, or Compound 3. In some embodiments, said modulator is Compound 1. In some embodiments, said modulator is Compound 2. In some embodiments, said modulator is Compound 3.

In certain embodiments, said modulator is orally bioavailable. In some embodiments, said oral bioavailability is at least 1%, at least 5%, at least 10%, at least 15%, at least 20%, at least 25%, at least 30%, at least 35%, at least 40%, or at least 45% relative to intraperitoneal administration. In some embodiments, said oral bioavailablity is at least 20%, at least 25%, at least 30%/c, at least 35%, at least 40%, or at least 45% relative to intraperitoneal administration.

In certain embodiments, said orally bioavailable modulator is further able to cross the blood-brain barrier.

In some embodiments, said modulator is an agonist with an EC₅₀ of less than 10 μM, of less than 1 μM, of less than 100 nM, or of less than 10 nM. In some embodiments, said modulator is an agonist with an EC₅₀ of less than a value selected from the interval of 10 nM to 10 μM. In some embodiments, said modulator is an agonist with an EC₅₀ of less than a value selected from the interal of 10 nM to 1 μM. In some embodiments, said modulator is an agonist with an EC₅₀ of less than a value selected from the interval of 10 nM to 100 nM. In certain embodiments, said EC50 is determined using an assay selected from the group consisting of: whole cell cAMP assay carried using transfected HEK293 cells expressing recombinant RUP43 GPCR polypeptide having the amino acid sequence of SEQ ID NO:2 or 6; and melanophore assay carried out using transfected melanophores expressing recombinant RUP43 GPCR polypeptide having the amino acid sequence of SEQ ID NO:2 or 6. In some embodiments, said modulator is an agonist with an EC₅₀ of less than 10 μM, of less than 1 μM, of less than 100 nM, or of less than 10 nM in said assay. In some embodiments, said modulator is an agonist with an EC₅₀ of less than 10 μM in said assay, of less than 9 μM in said assay, of less than 8 μM in said assay, of less than 7 μM in said assay, of less than 6 μM in said assay, of less than 5 μM in said assay, of less than 4 μM in said assay, of less than 3 μM in said assay, of less than 2 μM in said assay, of less than 1 μM in said assay, of less than 900 nM in said assay, of less than 800 nM in said assay, of less than 700 nM in said assay, of less than 600 nM in said assay, of less than 500 nM in said assay, of less than 400 nM in said assay, of less than 300 nM in said assay, of less than 200 nM in said assay, of less than 100 nM in said assay, of less than 90 nM in said assay, of less than 80 nM in said assay, of less than 70 nM in said assay, of less than 60 nM in said assay, of less than 50 nM in said assay, of less than 40 nM n said assay, of less than 30 nM in said assay, of less than 20 nM in said assay, or of less than 10 nM in said assay. In some embodiments, said modulator is an agonist with an EC₅₀ in said assay of less than a value selected from the interval of 10 nM to 10 μM. In some embodiments, said modulator is an agonist with an EC₅₀ in said assay of less than a value selected from the interval of 10 nM to 1 μM. In some embodiments, said modulator is an agonist with an EC₅₀ in said assay of less than a value selected from the interval of 10 nM to 100 nM.

In certain embodiments, said animal is a mammal. In certain embodiments, said mammal is a horse, cow, sheep, pig, cat, dog, rabbit, mouse, rat, or non-human primate. More preferred of human or animal is human.

In a twelfth aspect, the invention features a method of using a modulator of a RUP43 GPCR, said receptor comprising a GPR131 amino acid sequence, for the preparation of a medicament for the lowering of blood glucose concentration. In certain embodiments, the modulator is an agonist. In certain embodiments, the agonist of RUP43 GPCR is an agonist of GPR131 GPCR, where it is understood that GPR131 GPCR is endogenous RUP43GPCR.

The invention also features a method of using a modulator of a RUP43 GPCR, said receptor comprising a GPR131 amino acid sequence, for the preparation of a medicament for the prevention or treatment of a metabolic disorder. In certain embodiments, the modulator is an agonist. In certain embodiments, the agonist of RUP43 GPCR is an agonist of GPR131 GPCR, where it is understood that GPR131 GPCR is endogenous RUP43 GPCR. In certain embodiments, the metabolic disorder is selected from the group consisting of:

(a) diabetes;

(b) impaired glucose tolerance;

(c) insulin resistance; and

(d) hyperinsulinemia.

In some embodiments, diabetes is type 1 diabetes. In certain preferred embodiments, diabetes is type 2 diabetes. In certain embodiments, the metabolic disorder is diabetes. In certain embodiments, the metabolic disorder is type 1 diabetes. In certain embodiments, the metabolic disorder is type 2 diabetes. In certain embodiments, the metabolic disorder is impaired glucose tolerance. In certain embodiments, the metabolic disorder is insulin resistance. In certain embodiments, the metabolic disorder is hyperinsulinemia. In certain embodiments, the metabolic disorder is related to an elevated blood glucose concentration in the individual.

The invention also features a method of using a modulator of a RUP43 GPCR, said receptor comprising a GPR131 amino acid sequence, for the preparation of a medicament for the prevention or treatment of a complication of an elevated blood glucose concentration. In certain embodiments, the modulator is an agonist. In certain embodiments, the agonist of RUP43 GPCR is an agonist of GPR131 GPCR, where it is understood that GPR131 GPCR is endogenous RUP43 GPCR. In certain embodiments, the modulator is an agonist. In certain embodiments, the complication is selected from the group consisting of:

(a) Syndrome X;

(b) atherosclerosis;

(c) atheromatous disease;

(d) heart disease;

(e) hypertension;

(f) stroke;

(g) neuropathy;

(h) retinopathy;

(i) nephropathy; and

(j) peripheral vascular disease.

Heart disease includes, but is not limited to, cardiac insufficiency, coronary insufficiency, coronary artery disease, and high blood pressure. In certain embodiments, the complication is Syndrome X. In certain embodiments, the complication is atherosclerosis. In certain embodiments, the complication is atheromatous disease. In certain embodiments, the complication is heart disease. In certain embodiments, the complication is cardiac insufficiency. In certain embodiments, the complication is coronary insufficiency. In certain embodiments, the complication is coronary artery disease. In certain embodiments, the complication is high blood pressure. In certain embodiments, the complication is hypertension. In certain embodiments, the complication is stroke. In certain embodiments, the complication is neuropathy. In certain embodiments, the complication is retinopathy. In certain embodiments, the complication is neuropathy. In certain embodiments, the complication is peripheral vascular disease. In certain embodiments, the complication is polycystic ovary syndrome. In certain embodiments, the complication is hyperlipidemia.

In certain embodiments, the modulator is identifiable by a method according to a method of the first aspect. In certain embodiments, the modulator is identified according to a method of the first aspect. In certain embodiments, the modulator is not an antibody or an antigen-binding derivative thereof. In certain embodiments, the modulator is not a peptide. In certain embodiments, the modulator is a compound that stimulates glucose uptake in adipocytes obtained from a mammal. In certain embodiments, the modulator is a compound that stimulates glucose uptake in skeletal muscle cells obtained from a mammal. In certain embodiments, said modulator is selected from the group consisting of agonist, partial agonist, inverse agonist, and antagonist. In certain preferred embodiments, said modulator is an agonist. In certain embodiments, said agonist is a compound according to the second aspect.

In certain embodiments, said modulator is selective for the GPCR.

In some embodiments, said modulator is Compound 1, Compound 2, or Compound 3. In some embodiments, said modulator is Compound 1. In some embodiments, said modulator is Compound 2. In some embodiments, said modulator is Compound 3.

In certain embodiments, said modulator is orally bioavailable. In some embodiments, said oral bioavailability is at least 1%, at least 5%, at least 10%, at least 15%, at least 20%, at least 25%, at least 30%, at least 35%, at least 40%, or at least 45% relative to intraperitoneal administration. In some embodiments, said oral bioavailablity is at least 20%, at least 25%, at least 30.%, at least 35%, at least 40%, or at least 45% relative to intraperitoneal administration.

In certain embodiments, said orally bioavailable modulator is further able to cross the blood-brain barrier.

In some embodiments, said modulator is an agonist with an EC₅₀ of less than 10 μM, of less than 1 μM, of less than 100 nM, or of less than 10 nM. In some embodiments, said modulator is an agonist with an EC₅₀ of less than a value selected from the interval of 10 nM to 10 μM. In some embodiments, said modulator is an agonist with an EC₅₀ of less than a value selected from the interval of 10 nM to 1 μM. In some embodiments, said modulator is an agonist with an EC₅₀ of less than a value selected from the interval of 10 nM to 100 nM. In certain embodiments, said EC50 is determined using an assay selected from the group consisting of: whole cell cAMP assay carried using transfected HEK293 cells expressing recombinant RUP43 GPCR polypeptide having the amino acid sequence of SEQ ID NO:2 or 6; and melanophore assay carried out using transfected melanophores expressing recombinant RUP43 GPCR polypeptide having the amino acid sequence of SEQ ID NO:2 or 6. In some embodiments, said modulator is an agonist with an EC₅₀ of less than 10 μM, of less than 1 μM, of less than 100 nM, or of less than 10 nM in said assay. In some embodiments, said modulator is an agonist with an EC₅₀ of less than 10 μM in said assay, of less than 9 μM in said assay, of less than 8 μM in said assay, of less than 7 μM in said assay, of less than 6 μM in said assay, of less than 5 μM in said assay, of less than 4 μM in said assay, of less than 3 μM in said assay, of less than 2 μM in said assay, of less than 1 μM in said assay, of less than 900 nM in said assay, of less than 800 nM in said assay, of less than 700 nM in said assay, of less than 600 nM in said assay, of less than 500 nM in said assay, of less than 400 nM in said assay, of less than 300 nM in said assay, of less than 200 nM in said assay, of less than 100 nM in said assay, of less than 90 nM in said assay, of less than 80 nM in said assay, of less than 70 nM in said assay, of less than 60 nM in said assay, of less than 50 nM in said assay, of less than 40 nM n said assay, of less than 30 nM in said assay, of less than 20 nM in said assay, or of less than 10 nM in said assay. In some embodiments, said modulator is an agonist with an EC₅₀ in said assay of less than a value selected from the interval of 10 nM to 10 μM. In some embodiments, said modulator is an agonist with an EC₅₀ in said assay of less than a value selected from the interval of 10 nM to 1 μM. In some embodiments, said modulator is an agonist with an EC₅₀ in said assay of less than a value selected from the interval of 10nM to 100 nM.

In a thirteenth aspect, the invention features a method of modulating the activity of a RUP43 GPCR, said receptor comprising a GPR131 amino acid sequence, wherein said modulation is for lowering blood glucose in an individual in need of said modulation, comprising contacting said receptor with a therapeutically effective amount of a modulator of the receptor. In certain embodiments, said method comprises first performing a method according to the first aspect to thereby identify the modulator. In certain embodiments, the modulator is an agonist.

The invention also features a method of modulating the activity of a RUP43 GPCR, said receptor comprising a GPR131 amino acid sequence, wherein said modulation is for preventing or treating a metabolic disorder in an individual in need of said modulation, comprising contacting said receptor with a therapeutically effective amount of a modulator of the receptor. In certain embodiments, said method comprises first performing a method according to the first aspect to thereby identify the modulator. In certain embodiments, the modulator is an agonist. In certain embodiments, the metabolic disorder is selected from the group consisting of:

(a) diabetes;

(b) impaired glucose tolerance;

(c) insulin resistance; and

(d) hyperinsulinemia.

In some embodiments, diabetes is type 1 diabetes. In certain preferred embodiments, diabetes is type 2 diabetes. In certain embodiments, the metabolic disorder is diabetes. In certain embodiments, the metabolic disorder is type 1 diabetes. In certain embodiments, the metabolic disorder is type 2 diabetes. In certain embodiments, the metabolic disorder is impaired glucose tolerance. In certain embodiments, the metabolic disorder is insulin resistance. In certain embodiments, the metabolic disorder is hyperinsulinemia. In certain embodiments, the metabolic disorder is related to an elevated blood glucose concentration in the individual.

The invention also features a method of modulating the activity of a RUP43 GPCR, said receptor comprising a GPR131 amino acid sequence, wherein said modulation is for preventing or treating a complication of an elevated blood glucose concentration in an individual in need of said modulation, comprising contacting said receptor with a therapeutically effective amount of a modulator of the receptor. In certain embodiments, said method comprises first performing a method according to the first aspect to thereby identify the modulator. In certain embodiments, the modulator is an agonist. In certain embodiments, the modulator is an agonist. In certain embodiments, the complication is selected from the group consisting of:

(a) Syndrome X;

(b) atherosclerosis;

(c) atheromatous disease;

(d) heart disease;

(e) hypertension;

(f) stroke;

(g ) neuropathy;

(h) retinopathy;

(i) nephropathy; and

(j) peripheral vascular disease.

Heart disease includes, but is not limited to, cardiac insufficiency, coronary insufficiency, coronary artery disease, and high blood pressure. In certain embodiments, the complication is Syndrome X. In certain embodiments, the complication is atherosclerosis. In certain embodiments, the complication is atheromatous disease. In certain embodiments, the complication is heart disease. In certain embodiments, the complication is cardiac insufficiency. In certain embodiments, the complication is coronary insufficiency. In certain embodiments, the complication is coronary artery disease. In certain embodiments, the complication is high blood pressure. In certain embodiments, the complication is hypertension. In certain embodiments, the complication is stroke. In certain embodiments, the complication is neuropathy. In certain embodiments, the complication is retinopathy. In certain embodiments, the complication is neuropathy. In certain embodiments, the complication is peripheral vascular disease. In certain embodiments, the complication is polycystic ovary syndrome. In certain embodiments, the complication is hyperlipidemia.

In certain embodiments, the modulator is not an antibody or an antigen-binding derivative thereof. In certain embodiments, said modulator is not a peptide. In certain embodiments, the modulator is a compound that stimulates glucose uptake in adipocytes obtained from a mammal. In certain embodiments, the modulator is a compound that stimulates glucose uptake in skeletal muscle cells obtained from a mammal. In certain embodiments, said modulator is according to the third aspect. In certain embodiments, said modulator is selected from the group consisting of agonist, partial agonist inverse agonist, and antagonist. In certain preferred embodiments, said modulator is an agonist.

In certain embodiments, said modulator is selective for the GPCR.

In some embodiments, said modulator is Compound 1, Compound 2, or Compound 3. In some embodiments, said modulator is Compound 1. In some embodiments, said modulator is Compound 2. In some embodiments, said modulator is Compound 3.

In certain embodiments, said modulator is orally bioavailable. In some embodiments, said oral bioavailability is at least 1%, at least 5%, at leat 10%, at least 15%/, at least 20%, at least 25%, at least 30% at leat 35%, at least 40%, or at least 45% relative to intraperitoneal administration. In some embodiments, said oral bioavailablity is at least 20%, at least 25%, at least 30%, at least 35%, at least 40%, or at least 45% relative to intraperitoneal administration.

In certain embodiments, said orally bioavailable modulator is further able to cross the blood-brain barrier.

In some embodiments, said modulator is an agonist with an EC₅₀ of less than 10 μM, of less than 1 μM, of less than 100 nM, or of less than 10 nM. In some embodiments, said modulator is an agonist with an EC₅₀ of less than a value selected from the interval of 10 nM to 10 μM. In some embodiments, said modulator is an agonist with an EC₅₀ of less than a value selected from the inteval of 10 nM to 1 μM. In some embodiments, said modulator is an agonist with an EC₅₀ of less than a value selected from the interval of 10 nM to 100 nM. In certain embodiments, said EC50 is determined using an assay selected from the group consisting of: whole cells cAMP assay carried using transfected HEK293 cells expressing recombinant RUP43 GPCR polypeptide having the amino acid sequence of SEQ ID NO:2 or 6; and melanophore assay carried out using transfected melanophores expressing recombinant RUP43 GPCR polypeptide having the amino acid sequence of SEQ ID NO:2 or 6. In some embodiments, said modulator is an agonist with an EC₅₀ of less than 10 μM, of less than 1 μM, of less than 100 nM, or of less than 10 nM in said assay. In some embodiments, said modulator is an agonist with an EC₅₀ of less than 10 μM in said assay, of less than 9 μM in said assay, of less than 8 μM in said assay, of less than 7 μM in said assay, of less than 6 μM in said assay, of less than 5 μM in said assay, of less than 4 μM in said assay, of less than 3 μM in said assay, of less than 2 μM in said assay, of less than 1 μM in said assay, of less tan 900 nM in said assay, of less than 800 nM in said assay, of less than 700 nM in said assay, of less than 600 nM in said assay, of less than 500 nM in said assay, of less than 400 nM in said assay, of less than 300 nM in said assay, of less than 200 nM in said assay, of less than 100 nM in said assay, of less than 90 nM in said assay, of less than 80 nM in said assay, of less than 70 nM in said assay, of less than 60 nM in said assay, of less than 50 nM in said assay, of less than 40 nM n said assay, of less them 30 nM in said assay, of less than 20 nM in said assay, or of less than 10 nM in said assay. In some embodiments, said modulator is an agonist with an EC₅₀ in said assay of less than a value selected from the interval of 10 nM to 10 μM. In some embodiments, said modulator is an agonist with an EC₅₀ in said assay of less than a value selected from the interval of 10 nM to 1 μM. In some embodiments, said modulator is an agonist with an EC₅₀ in said assay of less than a value selected from the interval of 10 nM to 100 nM.

In certain embodiments, said contacting comprises oral administration of said modulator to said individual.

In certain embodiments, said individual is a mammal. In certain embodiments, said individual is a non-human mammal. In certain embodiments, said mammal is a horse, cow, sheep, pig, cat, dog, rabbit, mouse, rat, non-human primate or human. In certain embodiments, said mammal is a mouse, rat, non-human primate, or human. Most preferred is human.

In a fourteenth aspect, the invention features a method of lowering blood glucose in an individual in need of said lowering, comprising contacting a therapeutically effective amount of a modulator of a RUP43 GPCR with said receptor, said GPCR comprising a GPR131 amino acid sequence. In certain embodiments, said method comprises first performing a method according to the first aspect to thereby identify the modulator. In certain embodiments, the modulator is an agonist. The invention also features a method of preventing or treating a metabolic disorder in an individual in need of said prevention or treatment, comprising contacting a therapeutically effective amount of a modulator of a RUP43 GPCR with said receptor, said receptor comprising a GPR131 amino acid sequence. In certain embodiments, said method comprises first performing a method according to the first aspect to thereby identify the modulator. In certain embodiments, the modulator is an agonist. In certain embodiments, the metabolic disorder is selected from the group consisting of:

(a) diabetes;

(b) impaired glucose tolerance;

(c) insulin resistance; and

(d) hyperinsulinemia.

In some embodiments, diabetes is type 1 diabetes. In certain preferred embodiments, diabetes is type 2 diabetes. In certain embodiments, the metabolic disorder is diabetes. In certain embodiments, the metabolic disorder is type 1 diabetes. In certain embodiments, the metabolic disorder is type 2 diabetes. In certain embodiments, the metabolic disorder is impaired glucose tolerance. In certain embodiments, the metabolic disorder is insulin resistance. In certain embodiments, the metabolic disorder is hyperinsulinemia. In certain embodiments, the metabolic disorder is related to an elevated blood glucose concentration in the individual.

The invention also features a method of preventing or treating a complication of an elevated blood glucose concentration in an individual in need of said prevention or treatment, comprising contacting a therapeutically effective amount of a modulator of a RUP43 GPCR with said receptor, said receptor comprising a GPR131 amino acid sequence. In certain embodiments, said method comprises first performing a method according to the first aspect to thereby identify the modulator. In certain embodiments, the modulator is an agonist. In certain embodiments, the modulator is an agonist. In certain embodiments, the complication is selected from the group consisting of:

(a) Syndrome X;

(b) atherosclerosis;

(c) atheromatous disease;

(d) heart disease;

(e) hypertension;

(f) stroke;

(g) neuropathy;

(h) retinopathy;

(i) nephropathy; and

(j) peripheral vascular disease.

Heart disease includes, but is not limited to, cardiac insufficiency, coronary insufficiency, coronary artery disease, and high blood pressure. In certain embodiments, the complication is Syndrome X. In certain embodiments, the complication is atherosclerosis. In certain embodiments, the complication is atheromatous disease. In certain embodiments, the complication is heart disease. In certain embodiments, the complication is cardiac insufficiency. In certain embodiments, the complication is coronary insufficiency. In certain embodiments, the complication is coronary artery disease. In certain embodiments, the complication is high blood pressure. In certain embodiments, the complication is hypertension. In certain embodiments, the complication is stroke. In certain embodiments, the complication is neuropathy. In certain embodiments, the complication is retinopathy. In certain embodiments, the complication is neuropathy. In certain embodiments, the complication is peripheral vascular disease. In certain embodiments, the complication is polycystic ovary syndrome. In certain embodiments, the complication is hyperlipidemia.

In certain embodiments, the modulator is not an antibody or an antigen-binding derivative thereof. In certain embodiments, said modulator is not a peptide. In certain embodiments, the modulator is a compound that stimulates glucose uptake in adipocytes obtained from a mammal. In certain embodiments, the modulator is a compound that stimulates glucose uptake in skeletal muscle cells obtained from a mammal. In certain embodiments, said modulator is according to the third aspect. In certain embodiments, said modulator is selected from the group consisting of agonist, partial agonist, inverse agonist, and antagonist. In certain preferred embodiments, said modulator is an agonist.

In certain embodiments, said modulator is selective for the GPCR.

In some embodiments, said modulator is Compound 1, Compound 2, or Compound 3. In some embodiments, said modulator is Compound 1. In some embodiments, said modulator is Compound 2. In some embodiments, said modulator is Compound 3.

In certain embodiments, said modulator is orally bioavailable. In some embodiments, said oral bioavailability is at least 1%, at least 5%, at least 10%, at least 15%, at least 20%, at least 25%, at least 30%, at least 35%, at least 40%, or at least 45% relative to intraperitoneal administration. In some embodiments, said oral bioavaiiablity is at least 20%, at least 25%, at least 30%, at least 35%, at least 40%, or at least 45% relative to intraperitoneal administration.

In certain embodiments, said orally bioavailable modulator is further able to cross the blood-brain barrier.

In some embodiments, said modulator is an agonist with an EC₅₀ of less than 10 μM, of less than 1 μM, of less than 100 nM or of less than 10 nM. In some embodiments, said modulator is an agonist with an EC₅₀ of less than a value selected from the interval of 10 nM to 10 μM. In some embodiments, said modulator is an agonist with an EC₅₀ of less than a value selected from the interval of 10 nM to 1 μM. In some embodiments, said modulator is an agonist with an EC₅₀ of less than a value selected from the interval of 10 nM to 100 nM. In certain embodiments, said EC50 is determined using an assay selected from the group consisting of: whole cell cAMP assay carried using transfected HEK293 cells expressing recombinant RUP43 GPCR polypeptide having the amino acid sequence of SEQ ID NO:2 or 6; and melanophore assay carried out using transfected melanophores expressing recombinant RUP43 GPCR polypeptide having the amino acid sequence of SEQ ID NO:2 or 6. In some embodiments, said modulator is an agonist with an EC₅₀ of less than 10 μM, of less than 1 μM, of less than 100 nM, or of less than 10 nM in said assay. In some embodiments, said modulator is an agonist with an EC₅₀ of less than 10 μM in said assay, of less than 9 μM in said assay, of less than 8 μM in said assay, of less than 7 μM in said assay, of less than 6 μM in said assay, of less than 5 μM in said assay, of less than 4 μM in said assay, of less than 3 μM in said assay, of less than 2 μM in said assay, of less than 1 μM in said assay, of less than 900 nM in said assay, of less than 800 nM in said assay, of less than 700 nM in said assay, of less than 600 nM in said assay, of less than 500 nM in said assay, of less than 400 nM in said assay, of less than 300 nM in said assay, of less than 200 nM in said assay, of less than 100 nM in said assay, of less than 90 nM in said assay, of less than 80 nM in said assay, of less than 70 nM in said assay, of less thin 60 nM in said assay, of less than 50 nM in said assay, of less than 40 nM n said assay, of less than 30 nM in said assay, of less than 20 nM in said assay, or of less than 10 nM in said assay. In some embodiments, said modulator is an agonist with an EC₅₀ in said assay of less than a value selected from the interval of 10 nM to 10 μM. In some embodiments, said modulator is an agonist with an EC₅₀ in said assay of less than a value selected from the interval of 10 nM to 1 μM. In some embodiments, said modulator is an agonist with an EC₅₀ in said assay of less than a value selected from the interval of 10 nM to 100 nM.

In certain embodiments, said contacting comprises oral administration of said modulator to said individual.

In certain embodiments, said individual is a mammal. In certain embodiments, said individual is a non-human mammal. In certain embodiments, said mammal is a horse, cow, sheep, pig, cat, dog, rabbit, mouse, rat, non-human primate or human. In certain embodiments, said mammal is a mouse, rat, non-human primate, or human. Most preferred is human.

In a fifteenth aspect, the invention features a pharmaceutical or physiologically acceptable composition comprising, consisting essentially of, or consisting of a modulator a RUP43 GPCR, said receptor comprising a GPR131 amino acid sequence. In certain embodiments, said modulator is identifiable by performing a method according to the first aspect. In certain embodiments, said modulator is identified by performing a method according to the first aspect.

In certain embodiments, the modulator is not an antibody or an antigen-binding derivative thereof. In certain embodiments, said modulator is not a peptide. In certain embodiments, the modulator is a compound that stimulates glucose uptake in adipocytes obtained from a mammal. In certain embodiments, the modulator is a compound that stimulates glucose uptake in skeletal muscle cells obtained from a mammal. In certain embodiments, said modulator is according to the third aspect. In certain embodiments, said modulator is selected from the group consisting of agonist, partial agonist, inverse agonist, and antagonist. In certain preferred embodiments, said modulator is an agonist.

In certain embodiments, said composition is pharmaceutical. In certain embodiments, the pharmaceutical composition comprises the modulator of a RUP43 GPCR. In certain embodiments, the pharmaceutical composition consists essentially of the modulator of a RUP43 GPCR. In certain embodiments, the pharmaceutical composition consists of the modulator of a RUP43 GPCR.

In certain embodiments, said composition is physiologically acceptable. In certain embodiments, the physiologically acceptable composition comprises the modulator of a RUP43 GPCR. In certain embodiments, the physiologically acceptable composition consists essentially of the modulator of a RUP43 GPCR. In certain embodiments, the physiologically acceptable composition consists of the modulator of a RUP43 GPCR.

In certain embodiments, said modulator is selective for the GPCR.

In some embodiments, said modulator is Compound 1, Compound 2, or Compound 3. In some embodiments, said modulator is Compound 1. In some embodiments, said modulator is Compound 2. In some embodiments, said modulator is Compound 3.

In certain embodiments, said modulator is orally bioavailable. In some embodiments, said oral bioavailability is at least 1%, at least 5%, at least 10%, at least 15%, at least 20%, at least 25%, at least 30% at least 35%, at least 40%, or at least 45% relative to intraperitoneal administration. In some embodiments, said oral bioavailability is at least 20%, at least 25%, at least 30%, at least 35%, at least 40%, or at least 45% relative to intraperitoneal administration.

In certain embodiments, said orally bioavailable modulator is further able to cross the blood-brain barrier.

In some embodiments, said modulator is an agonist with an EC₅₀ of less than 10 μM, of less than 1 μM, of less than 100 nM, or of less than 10 nM. In some embodiments, said modulator is an agonist with an EC₅₀ of less than a value selected from the intal of 10 nM to 10 μM. In some embodiments, said modulator is an agonist with an EC₅₀ of less than a value selected from the interval of 10 nM to 1 μM. In some embodiments, said modulator is an agonist with an EC₅₀ of less than a value selected from the interval of 10 nM to 100 nM. In certain embodiments, said EC50 is determined using an assay selected from the group consisting of: whole cell cAMP assay carried using transfected HEK293 cells expressing recombinant RUP43 GPCR polypeptide having the amino acid sequence of SEQ ID NO:2 or 6; and melanophore assay carried out using transfected melanophores expressing recombinant RUP43 GPCR polypeptide having the amino acid sequence of SEQ ID NO:2 or 6. In some embodiments, said modulator is an agonist with an EC₅₀ of less than 10 μM, of less than 1 μM, of less than 100 nM or of less than 10 nM in said assay. In some embodiments, said modulator is an agonist with an EC₅₀ of less than 10 μM in said assay, of less than 9 μM in said assay, of less than 8 μM in said assay, of less than 7 μM in said assay, of less than 6 μM in said assay, of less than 5 μM in said assay, of less than 4 μM in said assay, of less than 3 μM in said assay, of less than 2 μM in said assay, of less than 1 μM in said assay, of less than 900 nM in said assay, of less than 800 nM in said assay, of less than 700 nM in said assay, of less than 600 nM in said assay, of less than 500 nM in said assay, of less than 400 nM in said assay, of less than 300 nM in said assay, of less than 200 nM in said assay, of less than 100 nM in said assay, of less than 90 nM in said assay, of less than 80 nM in said assay, of less than 70 nM in said assay, of less than 60 nM in said assay, of less than 50 nM in said assay, of less than 40 nM n said assay, of less than 30 nM in said assay, of less than 20 nM in said assay, or of less than 10 nM in said assay. In some embodiments, said modulator is an agonist with an EC₅₀ in said assay of less than a value selected from the interval of 10 nM to 10 μM. In some embodiments, said modulator is an agonist with an EC₅₀ in said assay of less than a value selected from the interval of 10 nM to 1 μM. In some embodiments, said modulator is an agonist with an EC₅₀ in said assay of less than a value selected from the interval of 10 nM to 100 nM.

In a sixteenth aspect, the invention features a method of lowering blood glucose concentration comprising providing or administering to an individual in need of said reduction said pharmaceutical or physiologically acceptable composition of the fifteenth aspect.

The invention also features a method of preventing or treating a metabolic disorder comprising providing or administering to an individual in need of said prevention or treatment said pharmaceutical or physiologically acceptable composition of the fifteenth aspect. In certain embodiments, the metabolic disorder is selected from the group consisting of:

(a) diabetes;

(b) impaired glucose tolerance;

(c) insulin resistance; and

(d) hyperinsulinemia.

In some embodiments, diabetes is type 1 diabetes. In certain preferred embodiments, diabetes is type 2 diabetes. In certain embodiments, the metabolic disorder is diabetes. In certain embodiments, the metabolic disorder is type 1 diabetes. In certain embodiments, the metabolic disorder is type 2 diabetes. In certain embodiments, the metabolic disorder is impaired glucose tolerance. In certain embodiments, the metabolic disorder is insulin resistance. In certain embodiments, the metabolic disorder is hyperinsulinemia. In certain embodiments, the metabolic disorder is related to an elevated blood glucose concentration in the individual.

The invention also features a method of preventing or treating a complication of an elevated blood glucose concentration comprising providing or administering to an individual in need of said prevention or treatment said pharmaceutical or physiologically acceptable composition of the fifteenth aspect. In certain embodiments, the complication is selected from the group consisting of:

(a) Syndrome X;

(b) atherosclerosis;

(c) atheromatous disease;

(d) heart disease;

(e) hypertension;

(f) stroke;

(g) neuropathy,

(h) retinopathy;

(i) nephropathy; and

(j) peripheral vascular disease.

Heart disease includes, but is not limited to, cardiac insufficiency, coronary insufficiency, coronary artery disease, and high blood pressure. In certain embodiments, the complication is Syndrome x. In certain embodiments, the complication is atherosclerosis. In certain embodiments, the complication is atheromatous disease. In certain embodiments, the complication is heart disease. In certain embodiments, the complication is cardiac insufficiency. In certain embodiments, the complication is coronary insufficiency. In certain embodiments, the complication is coronary artery disease. In certain embodiments, the complication is high blood pressure. In certain embodiments, the complication is hypertension. In certain embodiments, the complication is stroke. In certain embodiments, the complication is neuropathy. In certain embodiments, the complication is retinopathy. In certain embodiments, the complication is neuropathy. In certain embodiments, the complication is peripheral vascular disease. In certain embodiments, the complication is polycystic ovary syndrome. In certain embodiments, the complication is hyperlipidemia.

In certain embodiments, said modulator is an agonist.

In certain embodiments, a therapeutically effective amount of said pharmaceutical or physiologically acceptable composition is provided or administered to said individual.

In certain embodiments, said providing or administering of said pharmaceutical or physiologically acceptable composition is oral.

In certain embodiments, said individual is a mammal. In certain embodiments, said individual is a non-human mammal. In certain embodiments, said mammal is a horse, cow, sheep, pig, cat, dog, rabbit, mouse, rat, non-human primate or human. In certain embodiments, said mammal is a mouse, rat, non-human primate, or human. Most preferred is human.

In an seventeenth aspect, the invention features a modulator of a RUP43 GPCR, said receptor comprising a GPR131 amino acid sequence, for use in a method of treatment of the human or animal body by therapy. In certain embodiments, said modulator is identifiable by performing a method according to the first aspect. In certain embodiments, said modulator is identified by performing a method according to the first aspect.

In certain embodiments, the modulator is not an antibody or an antigen-binding derivative thereof. In certain embodiments, said modulator is not a peptide. In certain embodiments, the modulator is a compound that stimulates glucose uptake in adipocytes obtained from a mammal. In certain embodiments, the modulator is a compound that stimulates glucose uptake in skeletal muscle cells obtained from a mammal. In certain embodiments, said modulator is according to the third aspect. In certain embodiments, said modulator is selected from the group consisting of agonist, partial agonist, inverse agonist, and antagonist. In certain preferred embodiments, said modulator is an agonist.

In certain embodiments, said modulator is selective for the GPCR.

In some embodiments, said modulator is Compound 1, Compound 2, or Compound 3. In some embodiments, said modulator is Compound 1. In some embodiments, said modulator is Compound 2. In some embodiments, said modulator is Compound 3.

In certain embodiments, said modulator is orally bioavailable. In some embodiments, said oral bioavailability is at least 1%, at least 5%, at least 10%, at least 15%, at least 20%, at least 25%, at least 30%, at least 35%, at least 40%, or at least 45% relative to intraperitoneal administration. In some embodiments, said oral bioavailablity is at least 20%, at least 25%, at least 30%, at least 35%, at least 40%, or at least 45% relative to intraperitoneal administration.

In certain embodiments, said orally bioavailable modulator is further able to cross the blood-brain barrier.

In some embodiments, said modulator is an agonist with an EC₅₀ of less than 10 μM, of less than 1 μM, of less than 100 nM, or of less than 10 nM. In some embodiments, said modulator is an agonist with an EC₅₀ of less than a value selected from the interval of 10 nM to 10 μM. In some embodiments, said modulator is an agonist with an EC50 of less than a value selected from the interval of 10 nM to 1 μM. In some embodiments, said modulator is an agonist with an EC₅₀ of less than a value selected from the interval of 10 nM to 100 nM. In certain embodiments, said EC50 is determined using an assay selected from the group consisting of: whole cell cAMP assay carried using transfected HEK293 cells expressing recombinant RUP43 GPCR polypeptide having the amino acid sequence of SEQ ID NO:2 or 6; and melanophore assay carried out using transfected melanophores expressing recombinant RUP43 GPCR polypeptide having the amino acid sequence of SEQ ID NO:2 or 6. In some embodiments, said modulator is an agonist with an EC₅₀ of less than 10 μM, of less than 1 μM, of less than 100 nM, or of less than 10 nM in said assay. In some embodiments, said modulator is an agonist with an EC₅₀ of less than 10 μM in said assay, of less than 9 μM in said assay, of less than 8 μM in said assay, of less than 7 μM in said assay, of less than 6 μM in said assay, of less than 5 M in said assay, of less than 4 M in said assay, of less than 3 μM in said assay, of less than 2 μM in said assay, of less than 1 μM in said assay, of less than 900 nM in said assay, of less than 800 nM in said assay, of less than 700 nM in said assay, of less than 600 nM in said assay, of less than 500 nM in said assay, of less than 400 nM in said assay, of less than 300 nM in said assay, of less than 200 nM in said assay, of less than 100 nM in said assay, of less than 90 nM in said assay, of less than 80 nM in said assay, of less than 70 nM in said assay, of less than 60 nM in said assay, of less than 50 nM in said assay, of less than 40 nM n said assay, of less &fan 30 nM in said assay, of less than 20 nM in said assay, or of less than 10 nM in said assay. In some embodiments, said modulator is an agonist with an EC₅₀ in said assay of less than a value selected from the interval of 10 nM to 10 μM. In some embodiments, said modulator is an agonist with an EC₅₀ in said assay of less than a value selected from the interval of 10 nM to 1 μM. In some embodiments, said modulator is an agonist with an EC₅₀ in said assay of less than a value selected from the interval of 10 nM to 100 nM.

In certain embodiments, said animal is a mammal. In certain embodiments, said mammal is a horse, cow, sheep, pig, cat, dog, rabbit, mouse, rat, or non-human primate. More preferred of human or animal is human.

In an eighteenth aspect, the invention features a modulator of a RUP43 GPCR, said receptor comprising a GPR13 1 amino acid sequence, for use in a method of lowering blood glucose concentration in the human or animal body by therapy. In certain embodiments, said modulator is identifiable by performing a method according to the first aspect. In certain embodiments, said modulator is identified by performing a method according to the first aspect. In certain embodiments, the modulator is an agonist.

The invention also features a modulator of a RUP43 GPCR, said receptor comprising a GPR131 amino acid sequence, for use in a method of prevention of or treatment for a metabolic disorder in a human or animal body by therapy. In certain embodiments, said modulator is identifiable by performing a method according to the first aspect. In certain embodiments, said modulator is identified by performing a method according to the first aspect. In certain embodiments, the modulator is an agonist. In certain embodiments, the metabolic disorder is selected from the group consisting of:

(a) diabetes;

(b) impaired glucose tolerance;

(c) insulin resistance; and

(d) hyperinsulinemia.

In some embodiments, diabetes is type 1 diabetes. In certain preferred embodiments, diabetes is type 2 diabetes. In certain embodiments, the metabolic disorder is diabetes. In certain embodiments, the metabolic disorder is type 1 diabetes. In certain embodiments, the metabolic disorder is type 2 diabetes. In certain embodiments, the metabolic disorder is impaired glucose tolerance. In certain embodiments, the metabolic disorder is insulin resistance. In certain embodiments, the metabolic disorder is hyperinsulinemia. In certain embodiments, the metabolic disorder is related to an elevated blood glucose concentration in the individual.

The invention also features a modulator of a RUP43 GPCR, said receptor comprising a GPR131 amino acid sequence, for use in a method of prevention of or treatment for a complication of an elevated blood glucose concentration in a human or animal body by therapy. In certain embodiments, said modulator is identifiable by performing a method according to the first aspect. In certain embodiments, said modulator is identified by performing a method according to the first aspect. In certain embodiments, the modulator is an agonist. In certain embodiments, the complication is selected from the group consisting of:

(a) Syndrome X;

(b) atherosclerosis;

(c) atheromatous disease;

(d) heart disease;

(e) hypertension;

(f) stroke;

(g) neuropathy;

(h) retinopathy;

(i) nephropathy; and

(j) peripheral vascular disease.

Heart disease includes, but is not limited to, cardiac insufficiency, coronary insufficiency, coronary artery disease, and high blood pressure. In certain embodiments, the complication is Syndrome X. In certain embodiments, the complication is atherosclerosis. In certain embodiments, the complication is atheromatous disease. In certain embodiments, the complication is heart disease. In certain embodiments, the complication is cardiac insufficiency. In certain embodiments, the complication is coronary insufficiency. In certain embodiments, the complication is coronary artery disease. In certain embodiments, the complication is high blood pressure. In certain embodiments, the complication is hypertension. In certain embodiments, the complication is stroke. In certain embodiments, the complication is neuropathy. In certain embodiments, the complication is retinopathy. In certain embodiments, the complication is neuropathy. In certain embodiments, the complication is peripheral vascular disease. In certain embodiments, the complication is polycystic ovary syndrome. In certain embodiments, the complication is hyperlipidemia.

In certain embodiments, the modulator is not an antibody or an antigen-binding derivative thereof. In certain embodiments, said modulator is not a peptide. In certain embodiments, the modulator is a compound that stimulates glucose uptake in adipocytes obtained from a mammal. In certain embodiments, the modulator is a compound that stimulates glucose uptake in skeletal muscle cells obtained from a mammal. In certain embodiments, said modulator is according to the third aspect. In certain embodiments, said modulator is selected from the group consisting of agonist, partial agonist, inverse agonist, and antagonist. In certain preferred embodiments, said modulator is an agonist.

In certain embodiments, said modulator is selective for the GPCR.

In some embodiments, said modulator is Compound 1, Compound 2, or Compound 3. In some embodiments, said modulator is Compound 1. In some embodiments, said modulator is Compound 2. In some embodiments, said modulator is Compound 3.

In certain embodiments, said modulator is orally bioavailable. In some embodiments, said oral bioavailability is at least 1%, at least 5%, at least 10%, at least 15%, at least 20%, at least 25%, at least 30%, at least 35%, at least 40%, or at least 45% relative to intreperitoneal administration. In some embodiments, said oral bioavailablity is at least 20%, at least 25%, at least 30%, at least 35%, at least 40%, or at least 45% relative to intraperitoneal administration.

In certain embodiments, said orally bioavailable modulator is further able to cross the blood-brain barrier.

In some embodiments, said modulator is an agonist with an EC₅₀ of less than 10 μM, of less than 1 μM, of less than 100 nM, or of less than 10 nM. In some embodiments, said modulator is an agonist with an EC₅₀ of less than a value selected from the interval of 10 nM to 10 pμM. In some embodiments, said modulator is an agonist with an EC₅₀ of less than a value selected from the interval of 10 nM to 1 μM. In some embodiments, said modulator is an agonist with an EC₅₀ of less than a value selected from the interval of 10 nM to 100 nM. In certain embodiments, said EC50 is determined using an assay selected from the group consisting of: whole cell cAMP assay carried using transfected HEK293 cells expressing recombinant RUP43 GPCR polypeptide having the ammo acid sequence of SEQ ID NO:2 or 6; and melanophore assay carried out using transfected melanophores expressing recombinant RUP43 GPCR polypeptide having the amino acid sequence of SEQ ID NO:2 or 6. In some embodiments, said modulator is an agonist with an EC₅₀ of less than 10 μM, of less than 1 μM, of less than 100 nM, or of less than 10 nM in said assay. In some embodiments, said modulator is an agonist with an EC₅₀ of less than 10 μM in said assay, of less than 9 μM in said assay, of less than 8 μM in said assay, of less than 7 μM in said assay, of less than 6 μM in said assay, of less than 5 μM in said assay, of less than 4 μM in said assay, of less than 3 μM in said assay, of less than 2 μM in said assay, of less than 1 μM in said assay, of less than 900 nM in said assay, of less than 800 nM in said assay, of less than 700 nM in said assay, of less than 600 nM in said assay, of less than 500 nM in said assay, of less than 400 nM in said assay, of less than 300 nM in said assay, of less than 200 nM in said assay, of less than 100 nM in said assay, of less than 90 nM in said assay, of less than 80 nM in said assay, of less than 70 nM in said assay, of less than 60 nM in said assay, of less than 50 nM in said assay, of less than 40 nM n said assay, of less than 30 nM in said assay, of less than 20 nM in said assay, or of less than 10 nM in said assay. In some embodiments, said modulator is an agonist with an EC₅₀ in said assay of less than a value selected from the interval of 10 nM to 10 μM. In some embodiments, said modulator is an agonist with an EC₅₀ in said assay of less than a value selected from the interval of 10 nM to 1 μM. In some embodiments, said modulator is an agonist with an EC₅₀ in said assay of less than a value selected from the interval of 10 nM to 100 nM.

In certain embodiments, said animal is a mammal. In certain embodiments, said mammal is a horse, cow, sheep, pig, cat, dog, rabbit, mouse, rat, or non-human primate. More preferred of human or animal is human.

In a nineteenth aspect, the invention features a method of using a modulator of a RUP43 GPCR, said receptor comprising a GPR131 amino acid sequence, for the preparation of a medicament for the lowering of blood glucose concentration. In certain embodiments, said method comprises first performing a method according to the first aspect to thereby identify the modulator. In certain embodiments, the modulator is an agonist.

The invention also features a method of using a modulator of a RUP43 GPCR, said receptor comprising a GPR131 amino acid sequence, for the preparation of a medicament for the prevention or treatment of a metabolic disorder. In certain embodiments, said method comprises performing a method according to the first aspect to thereby identify a modulator. In certain embodiments, the modulator is an agonist. In certain embodiments, the metabolic disorder is selected from the group consisting of:

(a) diabetes;

(b) impaired glucose tolerance;

(c) insulin resistance; and

(d) hyperinsulinemia.

In some embodiments, diabetes is type 1 diabetes. In certain preferred embodiments, diabetes is type 2 diabetes. In certain embodiments, the metabolic disorder is diabetes. In certain embodiments, the metabolic disorder is type 1 diabetes. In certain embodiments, the metabolic disorder is type 2 diabetes. In certain embodiments, the metabolic disorder is impaired glucose tolerance. In certain embodiments, the metabolic disorder is insulin resistance. In certain embodiments, the metabolic disorder is hyperinsulinemia. In certain embodiments, the metabolic disorder is related to an elevated blood glucose concentration in the individual.

The invention also features a method of using a modulator of a RUP43 GPCR, said receptor comprising a GPR131 amino acid sequence, for the preparation of a medicament for the prevention or treatment of a complication of an elevated blood glucose concentration. In certain embodiments, said method comprises performing a method according to the first aspect to thereby identify a modulator. In certain embodiments, the modulator is an agonist. In certain embodiments, the complication is selected from the group consisting of:

(a) Syndrome X;

(b) atherosclerosis;

(c) atheromatous disease;

(d) heart disease;

(e) hypertension;

(f) stroke;

A) neuropathy;

(h) retinopathy;

(i) nephropathy; and

(j) peripheral vascular disease.

Heart disease includes, but is not limited to, cardiac insufficiency, coronary insufficiency, coronary artery disease, and high blood pressure. In certain embodiments, the complication is Syndrome X. In certain embodiments, the complication is atherosclerosis. In certain embodiments, the complication is atheromatous disease. In certain embodiments, the complication is heart disease. In certain embodiments, the complication is cardiac insufficiency. In certain embodiments, the complication is coronary insufficiency. In certain embodiments, the complication is coronary artery disease. In certain embodiments, the complication is high blood pressure. In certain embodiments, the complication is hypertension. In certain embodiments, the complication is stroke. In certain embodiments, the complication is neuropathy. In certain embodiments, the complication is retinopathy. In certain embodiments, the complication is neuropathy. In certain embodiments, the complication is peripheral vascular disease. In certain embodiments, the complication is polycystic ovary syndrome. In certain embodiments, the complication is hyperlipidemia.

In certain embodiments, the modulator is not an antibody or an antigen-binding derivative thereof. In certain embodiments, said modulator is not a peptide. In certain embodiments, the modulator is a compound that stimulates glucose uptake in adipocytes obtained from a mammal. In certain embodiments, the modulator is a compound that stimulates glucose uptake in skeletal muscle cells obtained from a mammal. In certain embodiments, said modulator is according to the third aspect. In certain embodiments, said modulator is selected from the group consisting of agonist, partial agonist, inverse agonist and antagonist. In certain preferred embodiments, said modulator is an agonist.

In certain embodiments, said modulator is selective for the GPCR.

In some embodiments, said modulator is Compound 1, Compound 2, or Compound 3. In some embodiments, said modulator is Compound 1. In some embodiments, said modulator is Compound 2. In some embodiments, said modulator is Compound 3.

In certain embodiments, said modulator is orally bioavailable. In some embodiments, said oral bioavailability is at least 1%, at least 5%Yo, at least 10%, at least 15%, at least 20%, at least 25%, at least 30%, at least 35%, at least 40%, or at least 45% relative to intraperitoneal administration In some embodiments, said oral bioavailablity is at least 20%, at least 25%, at least 30%, at least 35%, at least 40%, or at least 45% relative to intraperitoneal administration.

In certain embodiments, said orally bioavailable modulator is further able to cross the blood-brain barrier.

In some embodiments, said modulator is an agonist with an EC₅₀ of less than 10 μM, of less than 1 μM, of less than 100 nM, or of less than 10 nM. In some embodiments, said modulator is an agonist with an EC₅₀ of less than a value selected from the interval of 10 nM to 10 μM. In some embodiments, said modulator is an agonist with an EC₅₀ of less than a value selected from the interval of 10 nM to 1 μM. In some embodiments, said modulator is an agonist with an EC₅₀ of less than a value selected from the interval of 10 nM to 100 nM. In certain embodiments, said EC50 is determined using an assay selected from the group consisting of: whole cell cAMP assay carried using transfected HEK293 cells expressing recombinant RUP43 GPCR polypeptide having the amino acid sequence of SEQ ID NO:2 or 6; and melanophore assay carried out using transfected melanophores expressing recombinant RUP43 GPCR polypeptide having the amino acid sequence of SEQ ID NO:2 or 6. In some embodiments, said modulator is an agonist with an EC₅₀ of less than 10 μM, of less than 1 μM, of less than 100 nM, or of less than 10 nM in said assay. In some embodiments, said modulator is an agonist with an EC₅₀ of less than 10 μM in said assay, of less than 9 μM in said assay, of less than 8 μM in said assay, of less than 7 μM in said assay, of less than 6 μM in said assay, of less than 5 μM in said assay, of less than 4 M in said assay, of less than 3 μM in said assay, of less than 2 μM in said assay, of less than 1 μM in said assay, of less than 900 nM in said assay, of less than 800 nM in said assay, of less than 700 nM in said assay, of less than 600 nM in said assay, of less than 500 nM in said assay, of less than 400 nM in said assay, of less than 300 nM in said assay, of less than 200 nM in said assay, of less than 100 nM in said assay, of less than 90 nM in said assay, of less than 80 nM in said assay, of less than 70 nM in said assay, of less than 60 nM in said assay, of less than 50 nM in said assay, of less than 40 nM n said assay, of less than 30 nM in said assay, of less than 20 nM in said assay, or of less than 10 nM in said assay. In some embodiments, said modulator is an agonist with an EC₅₀ in said assay of less than a value selected from the interval of 10 nM to 10 μM. In some embodiments, said modulator is an agonist with an EC₅₀ in said assay of less than a value selected from the interval of 10 nM to 1 μM. In some embodiments, said modulator is an agonist with an EC₅₀ in said assay of less than a value selected from the interval of 10 nM to 100 nM.

In a twentieth aspect, the invention features a method of preparing a pharmaceutical or physiologically acceptable composition comprising admixing a compound according according to the second aspect and a carrier.

In certain embodiments, said composition is pharmaceutical. In certain embodiments, said composition is physiologically acceptable.

In certain embodiments, said compound is orally bioavailable. In some embodiments, said oral bioavailability is at least 1%, at least 5%, at least 10%, at least 15%, at least 20%, at least 25%, at least 30%, at least 35%, at least 40%, or at least 45% relative to intraperitoneal administration. In some embodiments, said oral bioavailablity is at least 20%, at least 25%, at least 30%, at least 35%, at least 40%, or at least 45% relative to intraperitoneal administration.

In certain embodiments, said orally bioavailable compound is further able to cross the blood-brain barrier.

In a twenty first aspect, the invention features a pharmaceutical or physiologically acceptable composition comprising, consisting essentially of, or consisting of a compound according to the second aspect.

In certain embodiments, said composition is pharmaceutical. In certain embodiments, the pharmaceutical composition comprises the compound according to the second aspect. In certain embodiments, the pharmaceutical composition consists essentially of the compound according to the second aspect. In certain embodiments, the pharmaceutical composition consists of the compound according to the second aspect.

In certain embodiments, said composition is physiologically acceptable. In certain embodiments, the physiologically acceptable composition comprises the compound according to the second aspect. In certain embodiments, the physiologically acceptable composition consists essentially of the compound according to the second aspect. In certain embodiments, the physiologically acceptable composition consists of the compound according to the second aspect.

In certain embodiments, said compound is orally bioavailable. In some embodiments, said oral bioavailability is at least 1%, at least 5%, at least 10%, at least 15%, at least 20%, at least 25%, at least 30%, at least 35%, at least 40%, or at least 45% relative to intraperitoneal administration. In some embodiments, said oral bioavailablity is at least 20%, at least 25%, at least 30%, at least 35%, at least 40%, or at least 45% relative to intraperitoneal administration.

In certain embodiments, said orally bioavailable compound is further able to cross the blood-brain barrier.

In a twenty-second aspect, the invention features a method of modulating the activity of a RUP43 GPCR, said receptor comprising a GPR131 amino acid sequence, wherein said modulation is for lowering blood glucose level in an individual in need of said modulation, comprising contacting said receptor with a therapeutically effective amount of a compound according to the second aspect or with a therapeutically effect amount of a pharmaceutical or physiologically acceptable composition according to the twenty-first aspect. In certain embodiments, said contacting is with a therapeutically effective amount of a compound according to the second aspect. In certain embodiments, said contacting is with a therapeutically effective amount of a pharmaceutical or physiologically acceptable composition according to the twenty-first aspect.

In certain embodiments, said compound is orally bioavailable. In some embodiments, said oral bioavailability is at least 1%, at least 5%, at least 10%, at least 15%, at least 20%, at least 25%, at least 30%, at least 35%, at least 40%, or at least 45% relative to intraperitoneal administration. In some embodiments, said oral bioavailablity is at least 20%, at least 25%, at least 30%, at least 35%, at least 40%, or at least 45% relative to intraperitoneal administration.

In certain embodiments, said orally bioavailable compound is further able to cross the blood-brain barrier.

In certain embodiments, said individual is a mammal. In certain emboodiments, said individual is a non-human mammal. In certain embodiments, said mammal is a horse, cow, sheep, pig, cat, dog, rabbit, mouse, rat, non-human primate or human. In certain embodiments, said mammal is a mouse, rat, non-human primate, or human. Most preferred is human.

In a twenty-third aspect, the invention features a method of modulating the activity of a RUP43 GPCR, said receptor comprising a GPR131 amino acid sequence, wherein said modulation is for preventing or treating a metabolic disorder in an individual in need of said modulation, comprising contacting said receptor with a therapeutically effective amount of a compound according to the second aspect or with a therapeutically effect amount of a pharmaceutical or physiologically acceptable composition according to the twenty-first aspect. In certain embodiments, said contacting is with a therapeutically effective amount of a compound according to the second aspect. In certain embodiments, said contacting is with a therapeutically effective amount of a pharmaceutical or physiologically acceptable composition according to the twenty-first aspect. In certain embodiments, the metabolic disorder is selected from the group consisting of:

(a) diabetes;

(b) impaired glucose tolerance;

(c) insulin resistance; and

(d) hyperinsulinemia.

In some embodiments, diabetes is type 1 diabetes. In certain preferred embodiments, diabetes is type 2 diabetes. In certain embodiments, the metabolic disorder is diabetes. In certain embodiments, the metabolic disorder is type 1 diabetes. In certain embodiments, the metabolic disorder is type 2 diabetes. In certain embodiments, the metabolic disorder is impaired glucose tolerance. In certain embodiments, the metabolic disorder is insulin resistance. In certain embodiments, the metabolic disorder is hyperinsulinemia. In certain embodiments, the metabolic disorder is related to an elevated blood glucose concentration in the individual.

The invention also features a method of modulating the activity of a RUP43 GPCR, said receptor comprising a GPR131 amino acid sequence, wherein said modulation is for preventing or treating a complication of an elevated blood glucose concentration in an individual in need of said modulation, comprising contacting said receptor with a therapeutically effective amount of a compound according to the second aspect or with a therapeutically effect amount of a pharmaceutical or physiologically acceptable composition according to the twenty-first aspect. In certain embodiments, said contacting is with a therapeutically effective amount of a compound according to the second aspect. In certain embodiments, said contacting is with a therapeutically effective amount of a pharmaceutical or physiologically acceptable composition according to the twenty-first aspect. In certain embodiments, the complication is selected from the group consisting of:

(a) Syndrome X;

(b) atherosclerosis;

(c) atheromatous disease;

(d) heart disease;

(e) hypertension;

(f) stroke;

(g) neuropathy;

(h) retinopathy;

(i) nephropathy; and

(j) peripheral vascular disease.

Heart disease includes, but is not limited to, cardiac insufficiency, coronary insufficiency, coronary artery disease, and high blood pressure. In certain embodiments, the complication is Syndrome X. In certain embodiments, the complication is atherosclerosis. In certain embodiments, the complication is atheromatous disease. In certain embodiments, the complication is heart disease. In certain embodiments, the complication is cardiac insufficiency. In certain embodiments, the complication is coronary insufficiency. In certain embodiments, the complication is coronary artery disease. In certain embodiments, the complication is high blood pressure. In certain embodiments, the complication is hypertension. In certain embodiments, the complication is stroke. In certain embodiments, the complication is neuropathy. In certain embodiments, the complication is retinopathy. In certain embodiments, the complication is neuropathy. In certain embodiments, the complication is peripheral vascular disease. In certain embodiments, the complication is polycystic ovary syndrome. In certain embodiments, the complication is hyperlipidemia.

In certain embodiments, said compound is orally bioavailable. In some embodiments, said oral bioavailability is at least 1%, at least 5%, at least 10%, at least 15%, at least 20%, at least 25%, at least 30%, at least 35%, at least 40%, or at least 45% relative to intraperitoneal administration. In some embodiments, said oral bioavailablity is at least 20%, at least 25%, at least 30%, at least 35%, at least 40%, or at least 45% relative to intraperitoneal administration.

In certain embodiments, said orally bioavailable compound is further able to cross the blood-brain barrier.

In certain embodiments, said individual is a mammal. In certain embodiments, said individual is a non-human mammal. In certain embodiments, said mammal is a horse, cow, sheep, pig, cat, dog, rabbit, mouse, rat, non-human primate or human. In certain embodiments, said mammal is a mouse, rat, non-human primate, or human. Most preferred is human.

In a twenty-fourth aspect, the invention features a method of lowering blood glucose concentration in an individual in need of said lowering, comprising contacting said receptor with a therapeutically effective amount of a compound according to the second aspect or with a therapeutically effective amount of a pharmaceutical or physiologically acceptable composition according to the twenty-first aspect with a RUP43 GPCR, said receptor comprising a GPR131 amino acid sequence. In certain embodiments, said contacting is with a therapeutically effective amount of a compound according to the second aspect. In certain embodiments, said contacting is with a therapeutically effective amount of a pharmaceutical or physiologically acceptable composition according to the twenty-first aspect.

In certain embodiments, said compound is orally bioavailable. In some embodiments, said oral bioavailability is at least 1%, at least 5%, at least 10%, at least 15%, at least 20%, at least 25%, at least 30%, at least 35%, at least 40%, or at least 45% relative to intraperitoneal administration. In some embodiments, said oral bioavailablity is at least 20%, at least 25%, at least 30%, at least 35%, at least 40%, or at least 45% relative to intraperitoneal administration. In certain embodiments, said orally bioavailable compound is further able to cross the blood-brain barrier.

In certain embodiments, said individual is a mammal. In certain embodiments, said individual is a non-human mammal. In certain embodiments, said mammal is a horse, cow, sheep, pig, cat, dog, rabbit, mouse, rat, non-human primate or human. In certain embodiments, said mammal is a mouse, rat, non-human primate, or human. Most prefered is human.

In a twenty-fifth aspect, the invention features a method of preventing or treating a metabolic disorder in an individual in need of said reducing, comprising contacting said receptor with a therapeutically effective amount of a compound according to the second aspect or with a therapeutically effect amount of a pharmaceutical or physiologically acceptable composition according to the twenty-first aspect with a RUP43 GPCR, said receptor comprising a GPR131 amino acid sequence. In certain embodiments, said contacting is with a therapeutically effective amount of a compound according to the second aspect. In certain embodiments, said contacting is with a therapeutically effective amount of a pharmaceutical or physiologically acceptable composition according to the twenty-first aspect. In certain embodiments, the metabolic disorder is selected from the group consisting of:

(a) diabetes;

(b) impaired glucose tolerance;

(c) insulin resistance; and

(d) hyperinsulinemia.

In some embodiments, diabetes is type 1 diabetes. In certain preferred embodiments, diabetes is type 2 diabetes. In certain embodiments, the metabolic disorder is diabetes. In certain embodiments, the metabolic disorder is type 1 diabetes. In certain embodiments, the metabolic disorder is type 2 diabetes. In certain embodiments, the metabolic disorder is impaired glucose tolerance. In certain embodiments, the metabolic disorder is insulin resistance. In certain embodiments, the metabolic disorder is hyperinsulinemia. In certain embodiments, the metabolic disorder is related to an elevated blood glucose concentration in the individual.

The invention also features a method of preventing or treating a complication of an elevated blood glucose concentration in an individual in need of said prevention or treatment, comprising contacting said receptor with a therapeutically effective amount of a compound according to the second aspect or with a therapeutically effect amount of a pharmaceutical or physiologically acceptable composition according to the twenty-first aspect with a RUP43 GPCR, said receptor comprising a GPR131 amino acid sequence. In certain embodiments, said contacting is with a therapeutically effective amount of a compound according to the second aspect. In certain embodiments, said contacting is with a therapeutically effective amount of a pharmaceutical or physiologically acceptable composition according to the twenty-first aspect. In certain embodiments, the complication is selected from the group consisting of:

(a) Syndrome X;

(b) atherosclerosis;

(c) atheromatous disease;

(d) heart disease;

(e) hypertension;

(f) stroke;

(g) neuropathy;

(h) retinopathy;

(i) nephropathy; and

(j) peripheral vascular disease.

Heart disease includes, but is not limited to, cardiac insufficiency, coronary insufficiency, coronary artery disease, and high blood pressure. In certain embodiments, the complication is Syndrome X. In certain embodiments, the complication is atherosclerosis. In certain embodiments, the complication is atheromatous disease. In certain embodiments, the complication is heart disease. In certain embodiments, the complication is cardiac insufficiency. In certain embodiments, the complication is coronary insufficiency. In certain embodiments, the complication is coronary artery disease. In certain embodiments, the complication is high blood pressure. In certain embodiments, the complication is hypertension. In certain embodiments, the complication is stroke. In certain embodiments, the complication is neuropathy. In certain embodiments, the complication is retinopathy. In certain embodiments, the complication is neuropathy. In certain embodiments, the complication is peripheral vascular disease. In certain embodiments, the complication is polycystic ovary syndrome. In certain embodiments, the complication is hyperlipidemia.

In certain embodiments, said compound is orally bioavailable. In some embodiments, said oral bioavailability is at least 1%, at least 5%, at least 10%, at least 15%, at least 20%, at least 25%, at least 30% at least 35%, at least 40%, or at least 45% relative to intraperitoneal administration. In some embodiments, said oral bioavailablity is at least 20%, at least 254, at least 30%, at least 35%, at least 40%, or at least 45% relative to intraperitoneal administration.

In certain embodiments, said orally bioavailable compound is further able to cross the blood-brain barrier.

In certain embodiments, said individual is a mammal. In certain embodiments, said individual is a non-human mammal. In certain embodiments, said mammal is a horse, cow, sheep, pig, cat, dog, rabbit, mouse, rat, non-human primate or human. In certain embodiments, said mammal is a mouse, rat, non-human primate, or human. Most preferred is human.

In a twenty-sixth aspect, the invention features a method of lowering blood glucose concentration comprising providing or administering to an individual in need of said reducing a compound according to the second aspect or with a therapeutically effective amount of a pharmaceutical or physiologically acceptable composition according to the twenty-first aspect. In certain embodiments, said providing or administering a compound is providing or administering a compound according to the second aspect. In certain embodiments, said providing or administering a pharmaceutical or physiologically acceptable composition is providing or administering a pharmaceutical or physiologically acceptable composition according to the twenty-first aspect.

In certain embodiments, said compound is orally bioavailable. In some embodiments, said oral bioavailability is at least 1%, at least 5%, at least 10%, at least 15%, at least 20%, at least 25%, at least 30% at least 35%, at least 40%, or at least 45% relative to intraperitoneal administration. In some embodiments, said oral bioavailablity is at least 20%, at least 25%, at least 30%, at least 35%, at least 40%, or at least 45% relative to intraperitoneal administration.

In certain embodiments, said orally bioavailable compound is further able to cross the blood-brain barrier.

In certain embodiments, said individual is a mammal. In certain embodiments, said individual is a non-human mammal. In certain embodiments, said mammal is a horse, cow, sheep, pig, cat, dog, rabbit, mouse, rat, non-human primate or human. In certain embodiments, said mammal is a mouse, rat) non-human primate, or human. Most preferred is human.

In a twenty-seventh aspect, the invention features a method of treating a metabolic disorder comprising providing or administering to an individual in need of said treating or preventing a compound according to the second aspect or with a therapeutically effective amount of a pharmaceutical or physiologically acceptable composition according to the twenty-first aspect. In certain embodiments, said providing or administering a compound is providing or administering a compound according to the second aspect. In certain embodiments, said providing or administering a pharmaceutical or physiologically acceptable composition is providing or administering a pharmaceutical or physiologically acceptable composition according to the twenty-first aspect. In certain embodiments, the metabolic disorder is selected from the group consisting of:

(a) diabetes;

(b) impaired glucose tolerance;

(c) insulin resistance; and

(d) hyperinsulinemia.

In some embodiments, diabetes is type 1 diabetes. In certain preferred embodiments, diabetes is type 2 diabetes. In certain embodiments, the metabolic disorder is diabetes. In certain embodiments, the metabolic disorder is type 1 diabetes. In certain embodiments, the metabolic disorder is type 2 diabetes. In certain embodiments, the metabolic disorder is impaired glucose tolerance. In certain embodiments, the metabolic disorder is insulin resistance. In certain embodiments, the metabolic disorder is hyperinsulinemia. In certain embodiments, the metabolic disorder is related to an elevated blood glucose concentration in the individual.

The invention also features a method of treating a complication of an elevated glucose concentration comprising providing or administering to an individual in need of said treating or preventing a compound according to the second aspect or with a therapeutically effective amount of a pharmaceutical or physiologically acceptable composition according to the twenty-first aspect. In certain embodiments, said providing or administering a compound is providing or administering a compound according to the second aspect. In certain embodiments, said providing or administering a pharmaceutical or physiologically acceptable composition is providing or administering a pharmaceutical or physiologically acceptable composition according to the twenty-first aspect. In certain embodiments, the complication is selected from the group consisting of:

(a) Syndrome X;

(b) atherosclerosis;

(c) atheromatous disease;

(d) heart disease;

(e) hypertension;

(f) stroke;

(g) neuropathy;

(h) retinopathy;

(i) nephropathy; and

(j) peripheral vascular disease.

Heart disease includes, but is not limited to, cardiac insufficiency, coronary insufficiency, coronary artery disease, and high blood pressure. In certain embodiments, the complication is Syndrome X. In certain embodiments, the complication is atherosclerosis. In certain embodiments, the complication is atheromatous disease. In certain embodiments, the complication is heart disease. In certain embodiments, the complication is cardiac insufficiency. In certain embodiments, the complication is coronary insufficiency. In certain embodiments, the complication is coronary artery disease. In certain embodiments, the complication is high blood pressure. In certain embodiments, the complication is hypertension. In certain embodiments, the complication is stroke. In certain embodiments, the complication is neuropathy. In certain embodiments, the complication is retinopathy. In certain embodiments, the complication is neuropathy. In certain embodiments, the complication is peripheral vascular disease. In certain embodiments, the complication is polycystic ovary syndrome. In certain embodiments, the complication is hyperlipidemia.

In certain embodiments, said compound is orally bioavailable. In some embodiments, said oral bioavailability is at least 1%, at least 5%, at least 10%, at least 15%, at least 20%, at least 25%, at least 30%, at least 35%, at least 40%, or at least 45% relative to intraperitoneal admninistration. In some embodiments, said oral bioavailablity is at least 20%, at least 25%, at least 30%, at least 35%, at least 40%/o, or at least 45% relative to intraperitoneal administration.

In certain embodiments, said orally bioavailable compound is further able to cross the blood-brain barrier.

In certain embodiments, said individual is a mammal. In certain embodiments, said individual is a non-human mammal. In certain embodiments, said mammal is a horse, cow, sheep, pig, cat, dog, rabbit, mouse, rat, non-human primate or human. In certain embodiments, said mammal is a mouse, rat, non-human primate, or human. Most preferred is human.

In a twenty-eighth aspect, the invention features a compound according to the second aspect for use in a method of treatment of the human or animal body by therapy.

In certain embodiments, said compound is orally bioavailable. In some embodiments, said oral bioavailability is at least 1%, at least 5%, at least 10%, at least 15%, at least 20%, at least 25%, at least 30%, at least 35%, at least 40%, or at least 45% relative to intraperitoneal administration. In some embodiments, said oral bioavailablity is at least 20%, at least 25%, at least 30%/o, at least 35%, at least 40%, or at least 45% relative to intraperitoneal administration.

In certain embodiments, said orally bioavailable compound is further able to cross the blood-brain barrier.

In certain embodiments, said animal is a mammal. In certain embodiments, said mammal is a horse, cow, sheep, pig, cat, dog, rabbit, mouse, rat, or non-human primate. More preferred of human or animal is human.

In a twenty-ninth aspect, the invention features a compound according to the second aspect for use in a method of lowering blood glucose concentration in the human or animal body by therapy.

In certain embodiments, said compound is orally bioavailable. In some embodiments, said oral bioavailability is at least 1%, at least 5%, at least 10%, at least 15%, at least 20%, at least 25%, at least 30%, at least 35%, at least 40%, or at least 45% relative to intraperitoneal administration. In some embodiments, said oral bioavailablity is at least 20%, at least 25%, at least 30%, at least 35%, at least 40%, or at least 45% relative to intraperitoneal administration.

In certain embodiments, said orally bioavailable compound is further able to cross the blood-brain barrier.

In certain embodiments, said animal is a mammal. In certain embodiments, said mammal is a horse, cow, sheep, pig, cat, dog, rabbit, mouse, rat, or non-human primate. More preferred of human or animal is human.

In a thirtieth aspect, the invention features a compound according to the second aspect for use in a method of prevention or treatment for a metabolic disorder in the human or animal body by therapy. In certain embodiments, the metabolic disorder is selected from the group consisting of:

(a) diabetes;

(b) impaired glucose tolerance;

(c) insulin resistance; and

(d) hyperinsulinemia.

In some embodiments, diabetes is type 1 diabetes. In certain preferred embodiments, diabetes is type 2 diabetes. In certain embodiments, the metabolic disorder is diabetes. In certain embodiments, the metabolic disorder is type 1 diabetes. In certain embodiments, the metabolic disorder is type 2 diabetes. In certain embodiments, the metabolic disorder is impaired glucose tolerance. In certain embodiments, the metabolic disorder is insulin resistance. In certain embodiments, the metabolic disorder is hyperinsulinemia. In certain embodiments, the metabolic disorder is related to an elevated blood glucose concentration in the individual.

The invention also features a compound according to the second aspect for use in a method of prevention or treatment for a complication of an elevated blood glucose concentration in the human or animal body by therapy. In certain embodiments, the complication is selected from the group consisting of:

(a) Syndrome X;

(b) atherosclerosis;

(c) atheromatous disease;

(d) heart disease;

(e) hypertension;

(f) stroke;

(g) neuropathy;

(h) retinopathy;

(i) nephropathy; and

(j) peripheral vascular disease.

Heart disease includes, but is not limited to, cardiac insufficiency, coronary insufficiency, coronary artery disease, and high blood pressure. In certain embodiments, the complication is Syndrome X. In certain embodiments, the complication is atherosclerosis. In certain embodiments, the complication is atheromatous disease. In certain embodiments, the complication is heart disease. In certain embodiments, the complication is cardiac insufficiency. In certain embodiments, the complication is coronary insufficiency. In certain embodiments, the complication is coronary artery disease. In certain embodiments, the complication is high blood pressure. In certain embodiments, the complication is hypertension. In certain embodiments, the complication is stroke. In certain embodiments, the complication is neuropathy. In certain embodiments, the complication is retinopathy. In certain embodiments, the complication is neuropathy. In certain embodiments, the complication is peripheral vascular disease. In certain embodiments, the complication is polycystic ovary syndrome. In certain embodiments, the complication is hyperlipidemia.

In certain embodiments, said compound is orally bioavailable. In some embodiments, said oral bioavailability is at least 1%, at least 5%, at least 10%, at least 15%, at least 20%, at least 25%, at least 30%, at least 35%, at least 40%, or at least 45% relative to intraperitoneal administration. In some embodiments, said oral bioavailablity is at least 20%, at least 25%, at least 30%, at least 35%, at least 40%, or at least 45% relative to intraperitoneal administration.

In certain embodiments, said orally bioavailable compound is further able to cross the blood-brain barrier.

In certain embodiments, said animal is a mammal. In certain embodiments, said mammal is a horse, cow, sheep, pig, cat, dog, rabbit, mouse, rat, or non-human primnate. More preferred of human or animal is human.

In a thirty-first aspect, the invention features a method of using a compound according to the second aspect for the preparation of a medicament for the reduction of blood glucose concentration.

In certain embodiments, said compound is orally bioavailable. In some embodiments, said oral bioavailability is at least 1%, at least 5%, at least 10%, at least 15%, at least 20%, at least 25%, at least 30%, at least 35%, at least 40%, or at least 45% relative to intraperitoneal administration. In some embodiments, said oral bioavaflablity is at least 20%, at least 25%, at least 30%, at least 35%, at least 40%, or at least 45% relative to intraperitoneal administration.

In certain embodiments, said orally bioavailable compound is further able to cross the blood-brain barrier.

In a thirty-second aspect, the invention features a method of using a compound according to the second aspect for the preparation of a medicament for the prevention of or treatment of a metabolic disorder. In certain embodiments, the metabolic disorder is selected from the group consisting of:

(a) diabetes;

(b) impaired glucose tolerance;

(c) insulin resistance; and

(d) hyperinsulinemia.

In some embodiments, diabetes is type 1 diabetes. In certain preferred embodiments, diabetes is type 2 diabetes. In certain embodiments, the metabolic disorder is diabetes. In certain embodiments, the metabolic disorder is type 1 diabetes. In certain embodiments, the metabolic disorder is type 2 diabetes. In certain embodiments, the metabolic disorder is impaired glucose tolerance. In certain embodiments, the metabolic disorder is insulin resistance. In certain embodiments, the metabolic disorder is hyperinsulinemia. In certain embodiments, the metabolic disorder is related to an elevated blood glucose concentration in the individual.

The invention also features a method of using a compound according to the second aspect for the preparation of a medicament for the prevention of or treatment of a complication of an elevated blood glucose concentration. In certain embodiments, the complication is selected from the group consisting of:

(a) Syndrome X;

(b) atherosclerosis;

(c) atheromatous disease;

(d) heart disease;

(e) hypertension;

(f) stroke;

(g) neuropathy;

(h) retinopathy;

(i) nephropathy; and

(j) peripheral vascular disease.

Heart disease includes, but is not limited to, cardiac insufficiency, coronary insufficiency, coronary artery disease, and high blood pressure. In certain embodiments, the complication is Syndrome X. In certain embodiments, the complication is atherosclerosis. In certain embodiments, the complication is atheromatous disease. In certain embodiments, the complication is heart disease. In certain embodiments, the complication is cardiac insufficiency. In certain embodiments, the complication is coronary insufficiency. In certain embodiments, the complication is coronary artery disease. In certain embodiments, the complication is high blood pressure. In certain embodiments, the complication is hypertension. In certain embodiments, the complication is stroke. In certain embodiments, the complication is neuropathy. In certain embodiments, the complication is retinopathy. In certain embodiments, the complication is neuropathy. In certain embodiments, the complication is peripheral vascular disease. In certain embodiments, the complication is polycystic ovary syndrome. In certain embodiments, the complication is hyperlipidemia.

In certain embodiments, said compound is orally bioavailable. In some embodiments, said oral bioavailability is at least 1%, at least 5%, at least 10%, at least 15%, at least 20%, at least 25%, at least 30%, at least 35%, at least 40%, or at least 45% relative to intraperitoneal administration. In some embodiments, said oral bioavailablity is at least 20%, at least 25%, at least 30%, at least 35%, at least 40%, or at least 45% relative to intraperitoneal administration.

In certain embodiments, said orally bioavallable compound is further able to cross the blood-brain barrier.

In a thirty-third aspect, the invention features a method of modulating a RUP43 GPCR, said receptor comprising a GPR131 amino acid sequence, comprising contacting said receptor with a compound according to the second aspect or with a pharmaceutical or physiologically acceptable composition according to the twenty-first aspect. In certain embodiments, said contacting is with a compound according to the second aspect. In certain embodiments, said contacting is with a pharmaceutical or physiologically acceptable composition according to the twenty-first aspect.

In certain embodiments, said compound is orally bioavailable. In some embodiments, said oral bioavailability is at least 1%, at least 5%, at least 10%, atleast 15%, at least 20%, at least 25%, at least 30%, at least 35%, at least 40%, or at least 45% relative to intraperitoneal administration. In some embodiments, said oral bioavailablity is at least 20%, at least 25%, at least 30%/o, at least 35%, at least 40%, or at least 45% relative to intraperitoneal administration.

In certain embodiments, said orally bioavailable compound is further able to cross the blood-brain barrier.

In a thirty-fourth aspect, the invention features a method of identifying one or more candidate compounds as a compound that binds to a RUP43 GPCR, said receptor comprising a GPR131 amino acid sequence, comprising the steps of:

(a) contacting the receptor with a detectably labeled known ligand of the GPCR in the presence or absence of the candidate compound; and

(b) determining whether the binding of said labeled ligand is inhibited in the presence of the candidate compound;

wherein said inhibition is indicative of the candidate compound being a compound that binds to a RUP43 GPCR.

In certain embodiments, the GPR131 amino acid sequence is selected from the group consisting of:
(a) the amino acid sequence of SEQ ID NO:2;
(b) amino acids 2-330 of SEQ ID NO:2;

(c) amino acids 2-330 of SEQ ID NO:2, with the proviso that the RUP43 G protein-coupled receptor does not comprise the methionine residue at amino acid position 1 of SEQ ID NO:2;

(d) the amino acid sequence of a G protein-coupled receptor encoded by a polynucleotide comprising a nucleic acid sequence, said nucleic acid sequence being obtainable by a process comprising performing PCR on a human DNA sample using primers SEQ ID NO:3 and SEQ ID NO:4;

(e) the amino acid sequence of SEQ ID NO:6;

(f) the amino acid sequence of a G protein-coupled receptor encoded by a polynucleotide comprising a nucleic acid sequence, said nucleic acid sequence being obtainable by a process comprising performing PCR on a human DNA sample using primers SEQ ID NO:7 and SEQ ID NO:8;

(g the amino acid sequence of SEQ ID NO:2 wherein the alanine at amino acid position 223 of SEQ ID NO:2 is substituted with lysine;

(h) amino acids 2-330 of SEQ ID NO:2 wherein the alanine at amino acid position 223 of SEQ ID NO:2 is substituted with lysine;

(i) amino acids 2-330 of SEQ ID NO:2 wherein the alanine at amino acid position 223 of SEQ ID NO:2 is substituted with lysine, with the proviso that the RUP43 G protein-coupled receptor does not comprise the methionine residue at amino acid position 1 of SEQ ID NO:2; and

(j) the amino acid sequence of a G protein-coupled receptor encoded by a polynucleotide that hybridizes under stringent conditions to the complement of SEQ ID NO: 1.

In certain embodiments, the RUP43 GPCR is recombinant. In certain embodiments, said contacting comprises contacting with a host cell or with membrane of a host cell that expresses the GPCR. In certain embodiments, said host cell that expresses the GPCR comprises an expression vector comprising a polynucleotide encoding the receptor.

In some embodiments, the GPR131 amino acid sequence is the amino acid sequence of SEQ ID NO:2. In some embodiments, the GPR131 amino acid sequence is a variant of the amino acid sequence of SEQ ID NO:2. In some embodiments, said variant of the amino acid sequence of SEQ ID NO:2 is an allelic variant or mammalian ortholog of said amino acid sequence. In some embodiments, said variant of the amino acid sequence of SEQ ID NO:2 is a non-endogenous, constitutively activated mutant of said amino acid sequence or of an allelic variant or mammalian ortholog of said amino acid sequence. In certain embodiments, said variant of the amino acid sequence of SEQ ID NO:2 is a biologically active fragment of said amino acid sequence or of an allelic variant or mammalian ortholog of said amino acid sequence. In certain embodiments, said biologically active fragment of the amino acid sequence of SEQ ID NO:2 or of an allelic variant or mammalian ortholog of said amino acid sequence is the amino acid sequence of SEQ ID NO:2 or of an allelic variant or mammalian ortholog of said amino acid sequence absent the N-terminal methionine. In certain embodiments, said variant of the amino acid sequence of SEQ ID NO:2 is at least about 75%, at least about 80%, at least about 85%, at least about 90%, at least about 91%, at least about 92%, at least about 93%, at least about 94%, at least about 95%, at least about 96%, at least about 97%, least about 98% or at least about 99% identical to the amino acid sequence of SEQ ID NO:2. In some embodiments, said variant of the amino acid sequence of SEQ ID NO:2 is at least about 90%, at least about 91%, at least about 92%, at least about 93%, at least about 94%, at least about 95%, at least about 96%, at least about 97%, at least about 98% or at least about 99% identical to the amino acid sequence of SEQ ID NO:2.

In certain embodiments, said membrane preparation is made by homogenization of the cells with a Brinkman Polytron™. In certain embodiments, said membrane preparation is made by homogenization with 3 bursts of 10-20 sec duration each of said polytron.

In certain embodiments, said candidate compound is not an antibody or derivative thereof.

In certain embodiments, said candidate compound is not a peptide.

In certain embodiments, said known ligand is a compound according to the second aspect.

In certain embodiments, said known ligand is a modulator according to the third aspect.

In certain embodiments, said known ligand is Compound 1, Compound 2, or Compound 3. In certain embodiments, said known ligand is Compound 1. In certain embodiments, said known ligand is Compound 2. In certain embodiments, said known ligand is Compound 3.

In certain embodiments, said known ligand is an antibody specific for the GPCR, or an antigen-binding derivative of the antibody.

In certain embodiments, said label is selected from the group consisting of:
(a) radioisotope;
(b) enzyme; and
(c) fluorophore.

In certain embodiments, said label is a radioisotope. In certain embodiments, said label is selected from the group consisting of ³H, ¹⁴C, ³⁵S, and ¹²⁵I.

Compound 1, Compound 2, or Compound 3 can be radiolabelled using techniques known in the art, infra. In certain embodiments, Compound 1, Compound 2, or Compound 3 is radiolabelled with ³H or ¹⁴C.

In other embodiments, said method further comprises the step of comparing the level of inhibition of binding of a labeled first known ligand by the candidate compound to a second level of inhibition of binding of said labeled first known ligand by a second ligand known to bind to the GPCR.

In a thirty-fifth aspect, the invention features a method for detecting ligands that bind to a RUP43 GPCR, said receptor comprising a GPR131 amino acid sequence, comprising the steps of: contacting a test ligand with a host cell or with membrane of a host cell that expresses said receptor, under conditions which permit interaction between said receptor and said test ligand; and

detecting a ligand bound to said receptor.

In certain embodiments, the GPR131 amino acid sequence is selected from the group consisting of:
(a) the amino acid sequence of SEQ m NO:2;
(b) amino acids 2-330 of SEQ ID NO:2;

(c) amino acids 2-330 of SEQ ID NO:2, with the proviso that the RUP43 G protein-coupled receptor does not comprise the methionine residue at amino acid position 1 of SEQ ID NO:2;

(d) the amino acid sequence of a G protein-coupled receptor encoded by a polynucleotide comprising a nucleic acid sequence, said nucleic acid sequence being obtainable by a process comprising performing PCR on a human DNA sample using primers SEQ ID NO:3 and SEQ ID NO:4;

(e) the amino acid sequence of SEQ ID NO:6;

(f) the amino acid sequence of a G protein-coupled receptor encoded by a polynucleotide comprising a nucleic acid sequence, said nucleic acid sequence being obtainable by a process comprising performing PCR on a human DNA sample using pruners SEQ ID NO:7 and SEQ ID NO:8;

(g) the amino acid sequence of SEQ ID NO:2 wherein the alanine at amino acid position 223 of SEQ ID NO:2 is substituted with lysine;

(h) amino acids 2-330 of SEQ ID NO:2 wherein the alanine at amino acid position 223 of SEQ ID NO:2 is substituted with lysine;

(i) amino acids 2-330 of SEQ ID NO:2 wherein the alanine at amino acid position 223 of SEQ ID NO:2 is substituted with lysine, with the proviso that the RUP43 G protein-coupled receptor does not comprise the methionine residue at amino acid position 1 of SEQ ID NO:2; and

(j) the amino acid sequence of a G protein-coupled receptor encoded by a polynucleotide that hybridizes under stringent conditions to the complement of SEQ ID NO:1.

In some embodiments, the GPR131 amino acid sequence is the amino acid sequence of SEQ ID NO:2. In some embodiments, the GPR131 amino acid sequence is a variant of the amino acid sequence of SEQ ID NO:2. In some embodiments, said variant of the amino acid sequence of SEQ ID NO:2 is an allelic variant or mammalian ortholog of said amino acid sequence. In some embodiments, said variant of the amino acid sequence of SEQ ID NO:2 is a non-endogenous, constitutively activated mutant of said amino acid sequence or of an allelic variant or mammalian ortholog of said amino acid sequence. In certain embodiments, said variant of the amino acid sequence of SEQ ID NO:2 is a biologically active fragment of said amino acid sequence or of an allelic variant or mammalian ortholog of said amino acid sequence. In certain embodiments, said biologically active fragment of the amino acid sequence of SEQ ID NO:2 or of an allelic variant or mammalian ortholog of said amino acid sequence is the amino acid sequence of SEQ ID NO:2 or of an allelic variant or mammalian ortholog of said amino acid sequence absent the N-terminal methionine. In certain embodiments, said variant of the amino acid sequence of SEQ ID NO:2 is at least about 75%, at least about 80%, at least about 850, at least about 90%, at least about 91%, at least about 92%, at least about 93%, at least about 94%, at least about 95%, at least about 96%, at least about 97%, at least about 98% or at least about 99% identical to the amino acid sequence of SEQ ID NO:2. In some embodiments, said variant of the amino acid sequence of SEQ ID NO:2 is at least about 90%, at least about 91%, at least about 92%, at least about 93%, at least about 94%, at least about 95%, at least about 96%, at least about 97%, at least about 98% or at least about 99% identical to the amino acid sequence of SEQ ID NO:2.

In certain embodiments, the RUP43 GPCR is recombinant. In certain embodinents, said contacting comprises contacting with a host cell or with membrane of a host cell that expresses the GPCR. In certain embodiments, said host cell that expresses the GPCR comprises an expression vector comprising a polynucleotide encoding the receptor.

In certain embodiments, said test ligand is not an antibody or an antigen-binding derivative thereof.

In certain embodiments, said test ligand is not a peptide.

In certain embodiments, said membrane preparation is made by homogenization of the cells with a Brinkman Polytron™. In certain embodiments, said membrane preparation is made by homogenization with 3 bursts of 10-20 sec duration each of said polytron.

In certain embodiments, said test ligand is labeled. In certain embodiments, said label is a radioisotope. In certain embodiments, said label is selected from the group consisting of ³H, ¹⁴C, ³⁵S, and ¹²⁵I.

Applicant reserves the right to exclude any one or more candidate compounds from any of the embodiments of the invention. Applicant also reserves the right to exclude any one or more modulators from any of the embodiments of the invention. Applicant further reserves the right to exclude any polynucleotide or polypeptide from any of the embodiments of the invention. Applicant additionally reserves the right to exclude any metabolic disorder or any complication of elevated blood glucose concentration. It is also expressly contemplated that metabolic disorders of the invention can be included in an embodiment either individually or in any combination. It is also expressly contemplated that complications of elevated blood glucose concentration of the invention can be included in an embodiment either individually or in any combination.

Throughout this application, various publications, patents and published patent applications are cited. The disclosures of these publications, patents and published patent applications referenced in this application are hereby incorporated by reference in their entirety into the present disclosure. Citation herein by Applicant of a publication, patent, or published patent application is not an admission by Applicant of said publication, patent, or published patent application as prior art.

Modifications and extension of the disclosed inventions that are within the purview of the skilled artisan are encompassed within the above disclosure and the claims that follow.

### BRIEF DESCRIPTION OF THE DRAWINGS

FIG. 1. By way of example and not limitation, FIG. 1 depicts results from a primary screen of candidate compounds against a “target receptor” which is a Gsα Fusion Protein construct of an endogenous, constitutively active Gs-coupled GPCR unrelated to RUP43. Results for “Compound A” are provided in well A2. Results for “Compound “B” are provided in well G9. (See, Example 7.)

FIG. 2. RT-PCR analysis of RUP43 expression by adipocytes and skeletal muscle cells. Human and mouse adipocytes express RUP43. Human and mouse skeletal muscle cells express RUP43. (See, Example 11.)

FIG. 3. Endogenous RUP43 couples to Gs. (See, Example 14.)

FIG. 4. Identification of Compound 1 as an agonist of RUP43. (See, Example 15.)

FIG. 5. Identification of Compound 2 as an agonist of RUP43. (See, Example 16.)

FIG. 6. Compound 2 stimulates glucose uptake in mouse 3T3L1 adipocytes by Compound 2. (See, Example 18.)

FIG. 7. Compound 2 enhances insulin-stimulated glucose uptake in mouse 3T3L1 adipocytes. (See, Example 19.)

FIG. 8. Compound 2 stimulates glucose uptake in primary human adipocytes. (See, Example 20.)

FIG. 9. Compound 2 stimulates glucose uptake in rat L6 myoblast cells. (See, Example 21.)

FIG. 10. Compound 2 enhances insulin-stimulated glucose uptake in rat L6 myoblast cells. (See, Example 22.)

FIG. 11. Compound 2 stimulates glucose uptake in primary human skeletal muscle cells. (See, Example 23.)

### DETAILED DESCRIPTION

Definitions

The scientific literature that has evolved around receptors has adopted a number of terms to refer to ligands having various effects on receptors. For clarity and consistency, the following definitions will be used throughout this patent document. To the extent that these definitions conflict with other definitions for these terms, the following definitions shall control:

AGONISTS shall mean materials (e.g., ligands, candidate compounds) that activate an intracellular response when they bind to the receptor. In some embodiments, AGONISTS are those materials not previously known to activate the intracellular response when they bind to the receptor (e.g. to enhance GTPγS binding to membranes or to elevate intracellular cAMP level). In some embodiments, AGONISTS are those materials not previously known to stimulate glucose uptake in adipocytes or in skeletal muscle cells obtained from a mammal when they bind to the receptor.

AMINO ACID ABBREVIATIONS used herein are set out in Table A:

TABLE A

| | | |
|---|---|---|
| ALANINE | ALA | A |
| ARGININE | ARG | R |
| ASPARAGINE | ASN | N |
| ASPARTIC ACID | ASP | D |
| CYSTEINE | CYS | C |
| GLUTAMIC ACID | GLU | E |
| GLUTAMINE | GLN | Q |
| GLYCINE | GLY | G |
| HISTIDINE | HIS | H |
| ISOLEUCINE | ILE | I |
| LEUCINE | LEU | L |
| LYSINE | LYS | K |
| METHIONINE | MET | M |
| PHENYLALANINE | PHE | F |
| PROLINE | PRO | P |
| SERINE | SER | S |
| THREONINE | THR | T |
| TRYPTOPHAN | TRP | W |
| TYROSINE | TYR | Y |
| VALINE | VAL | V |

ANTAGONISTS shall mean materials (e.g., ligands, candidate compounds) that competitively bind to the receptor at the same site as the agonists but which do not activate an intracellular response, and can thereby inhibit the intracellular responses elicited by agonists. ANTAGONISTS do not diminish the baseline intracellular response in the absence of an agonist. In some embodiments, ANTAGONISTS are those materials not previously known to compete with an agonist to inhibit the cellular response when they bind to the receptor, e.g. wherein the cellular response is GTPγS binding to membranes or the elevation of intracellular cAMP level.

ANTIBODIES are intended herein to encompass monoclonal antibodies and polyclonal antibodies. Antibodies are further intended to encompass IgG, IgA, IgD, IgE, and IgM Antibodies include whole antibodies, including single-chain whole antibodies, and antigen binding fragments thereof, including Fab, Fab′, F(ab)2 and F(ab′)2. Antibodies may be from any animal origin. Preferably, antibodies are human, murine, rabbit, goat, guinea pig, hamster, camel, donkey, sheep, horse or chicken. Preferably antibodies have binding affinities with a dissociation constant or Kd value less than 5×10⁻⁶M, 10⁻⁶M, 5×10⁻⁷M, 10⁻⁷M, 5×10⁻⁸M, 10⁻⁸M, 5×10⁻⁹M, 10⁻⁹M, 5×10⁻¹⁰M, 10⁻¹⁰M, 5×10⁻¹¹M, 10⁻¹¹M, 5×10⁻¹²M, 10⁻¹²M, 5×10⁻¹³M, 10⁻¹³M, 5×10⁻¹⁴M 10^{31 14}M, 5×10⁻¹⁵M and 10⁻¹⁵M. Antibodies of the present invention may be prepared by any suitable method known in the art. Derivatives of antibodies are intended to encompass, but not be limited to, antigen-binding fragments.

BIOLOGICALLY ACTIVE FRAGMENT of a GPCR polypeptide or amino acid sequence shall mean a fragment of the polypeptide or amino acid sequence having structural and biochemical functions of a naturally occurring GPCR. In certain embodiments, the biologically active fragment couples to a G protein. In certain embodiments, the biologically active fragment binds to an endogenous ligand.

CANDIDATE COMPOUND shall mean a molecule (for example, and not limitation, a chemical compound) that is amenable to a screening technique.

CHEMICAL GROUP, MOIETY OR RADICAL:

The term “C₁₋₄ alkyl” denotes a straight or branched carbon radical containing the number of carbons as indicated, for examples, in some embodiments, alkyl is a “C₁₋₄ alkyl” and the group contains 1 to 4 carbons, in still other embodiments, alkyl is a “C₂₋₆ alkyl” and the group contains 2 to 6 carbons. In some embodiments alkyl contains 1 to 3 carbons, some embodiments contain 1 to 2 carbons, and some embodiments contain 1 carbon. Examples of an alkyl include, but not limited to, methyl ethyl, n-propyl, iso-propyl, n-butyl, iso-butyl, t-butyl, sec-butyl, n-pentyl, iso-pentyl, sec-pentyl, neo-pentyl, hexyl, iso-hexyl, sec-hexyl, neo-hexyl, and the like.

The term “halogen” or “halo” denotes to a fluoro, chloro, bromo or iodo group.

CODON shall mean a grouping of three nucleotides (or equivalents to nucleotides) which generally comprise a nucleoside [adenosine (A), guanosine (G), cytidine (C), uridine (U) and thymidine (T)] coupled to a phosphate group and which, when translated, encodes an amino acid.

COMPOSITION means a material comprising at least one component. A “pharmaceutical composition” is an example of a composition.

COMPOUND EFFICACY shall mean a measurement of the ability of a compound to inhibit or stimulate receptor functionality; i.e. the ability to activate/inhibit a signal transduction pathway, in contrast to receptor binding affinity. Exemplary means of detecting compound efficacy are disclosed in the Example section of this patent document.

COMPRISING, CONSISTING ESSENTIALLY OF, and CONSISTING OF are defined herein according to their standard meaning. A defined meaning set forth in the M.P.E.P. controls over a defined meaning in the art and a defined meaning set forth in controlling Federal Circuit case law controls over a meaning set forth in the M.P.E.P.

CONSTITUTIVELY ACTIVE RECEPTOR shall mean a receptor stabilized in an active state by means other than through binding of the receptor to its ligand or a chemical equivalent thereof. A CONSTITUTIVELY ACTIVE RECEPTOR may be endogenous or non-endogenous.

CONSTITUTIVELY ACTIVATED RECEPTOR shall mean an endogenous receptor that has been modified so as to be constitutively active.

CONSTITUTIVE RECEPTOR ACTIVATION shall mean activation of a receptor in the absence of binding to its ligand or a chemical equivalent thereof.

CONTACT or CONTACTING shall mean bringing at least two moieties together, whether in an in vitro system or an in vivo system.

DECREASE is used to refer to a reduction in a measurable quantity and is used synonymously with the terms “reduce”, “diminish”, “lower”, and “lessen”.

ELEVATED BLOOD GLUCOSE CONCENTRATION shall mean a fasting blood glucose concentration in a mammal greater than the normal fasting blood glucose concentration for the mammal. By way of example, normal human fasting blood glucose concentration is less than 100 mg/dl. As used herein, an elevated human blood glucose concentration is a fasting blood glucose concentration of 100 mg/dl or greater. By way of illustration and not limitation, an elevated blood glucose concentration encompasses hyperglycemia.

ENDOGENOUS shall mean a material that a mammal naturally produces. ENDOGENOUS in reference to, for example and not limitation, the term “receptor,” shall mean that which is naturally produced by a mammal (for example, and not limitation, a human). ENDOGENOUS shall be understood to encompass allelic variants of a gene as well as the allelic polypeptide variants so encoded As used herein, “endogenous GPCR” and “native GPCR” are used interchangeably. By contrast the term NON-ENDOGENOUS in this context shall mean that which is not naturally produced by a mammal (for example, and not limitation, a human). For example, and not limitation, a receptor which is not constitutively active in its endogenous form, but when manipulated becomes constitutively active, is most preferably referred to herein as a “non-endogenous, constitutively activated receptor.”

EXPRESSION VECTOR is defined herein as a DNA sequence that is required for the transcription of cloned DNA and the translation of the transcribed mRNAs in an appropriate host cell recombinant for said EXPRESSION VECTOR. An appropriately constructed EXPRESSION VECTOR should contain an origin of replication for autonomous replication in host cells, selectable markers, a limited number of useful restriction enzyme sites, a potential for high copy number, and active promoters. Said cloned DNA to be transcribed is operably linked to a constitutively or conditionally active promoter within said expression vector. By way of illustration and not limitation, pCMV is an expression vector.

G PROTEIN COUPLED RECEPTOR FUSION PROTEIN and GPCR FUSION PROTEIN, in the context of the invention disclosed herein, each mean a non-endogenous protein comprising an endogenous, constitutively active GPCR or a non-endogenous, constitutively activated GPCR fused to at least one G protein, most preferably the alpha (a) subunit of such G protein (this being the subunit that binds GTP), with the G protein preferably being of the same type as the G protein that naturally couples with endogenous GPCR. For example, and not limitation, in an endogenous state, if the G protein “Gsα” is the predominate G protein that couples with the GPCR, a GPCR Fusion Protein based upon the specific GPCR would be a non-endogenous protein comprising the GPCR fused to Gsα; in some circumstances, as will be set forth below, a non-predominant G protein can be fused to the GPCR. The G protein can be fused directly to the C-terminus of the constitutively active GPCR or there may be spacers between the two.

HOST CELL shall mean a cell capable of having a vector incorporated therein. In certain embodiments, the vector is an expression vector. Exemplary host cells include but are not limited to 293, 293T, CHO, MCB3901, and COS-7 cells, as well as melanophore cells.

IN NEED OF PREVENTION OR TREATMENT as used herein refers to a judgement made by a caregiver (e.g. physician, nurse, nurse practitioner, etc. in the case of humans; veterinarian in the case of animals, including non-human mammals) that an individual or animal requires or will benefit from treatment. This judgement is made based on a variety of factors that are in the realm of a caregiver's expertise, but that include the knowledge that the individual or animal is ill, or will be ill, as the result of a condition that is treatable by the compounds of the invention.

INDIVIDUAL as used herein refers to any animal, including mammals, preferably mice, rats, other rodents, rabbits, dogs, cats, swine, cattle, sheep, horses, or primates, and most preferably humans.

INHIBIT or INHIBITING, in relationship to the term “response” shall mean that a response is decreased or prevented in the presence of a compound as opposed to in the absence of the compound.

IMPAIRED GLUCOSE TOLERANCE (IGT) as used herein is intended to indicate that condition associated with insulin-resistance that is intermediate between frank, type 2 diabetes and normal glucose tolerance (NGT). IGT is diagnosed by a procedure wherein an affected person's postprandial glucose response is determined to be abnormal as assessed by 2-hour postprandial plasma glucose levels. In this test, a measured amount of glucose is given to the patient and blood glucose levels are measured at regular intervals, usually every half hour for the first two hours and every hour thereafter. In a “normal” or non-IGT individual glucose levels rise during the first two hours to a level less than 140 mg/dl and then drop rapidly. In an IGT individual, the blood glucose levels are higher and the drop-off level is, at a slower rate.

INSULIN RESISTANCE as used herein is intended to encompass the usual diagnosis of insulin resistance made by any of a number of methods, including but not restricted to: the intravenous glucose tolerance test or measurement of the fasting insulin level. It is well known that there is an excellent correlation between the height of the fasting insulin level and the degree of insulin resistance. Therefore, one could use elevated fasting insulin levels as a surrogate maker for insulin resistance for the purpose of identifying which normal glucose tolerance (NGT) individuals have insulin resistance. A diagnosis of insulin resistance can also be made using the euglycemic glucose clamp test.

lNVERSE AGONISTS shall mean materials (e.g., ligand, candidate compound) that bind either to the endogenous form or to the constitutively activated form of the receptor so as to reduce the baseline intracellular response of the receptor observed in the absence of agonists.

ISOLATED shall mean that the material is removed from its original environment (e.g., the natural environment if it is naturally occurring). For example, a naturally occurring polynucleotide or polypeptide present in a living animal is not isolated, but the same polynucleotide or DNA or polypeptide, separated from some or all of the coexisting materials in the natural system, is isolated. Such a polynucleotide could be part of a vector and/or such a polynucleotide or polypeptide could be part of a composition, and still be isolated in that the vector or composition is not part of its natural environment.

LIGAND shall mean a molecule that specifically binds to a GPCR. A ligand may be, for example, a polypeptide, a lipid, a small molecule, an antibody. An endogenous ligand is a ligand that is an endogenous, natural ligand for a native GPCR. A ligand may be a GPCR “antagonist”, “agonist”, “partial agonist”, or “inverse agonist”, or the like.

As used herein, the terms MODULATE or MODIFY are meant to refer to an increase or decrease in the amount, quality, or effect of a particular activity, function or molecule. By way of illustration and not limitation, agonists, partial agonists, inverse agonists, and antagonists of a G protein-coupled receptor are modulators of the receptor.

PARTIAL AGONISTS shall mean materials (e.g., ligands, candidate compounds) that activate the intracellular response when they bind to the receptor to a lesser degree/extent than do full agonists.

PHARMACEUTICAL COMPOSITION shall mean a composition comprising at least one active ingredient, whereby the composition is amenable to investigation for a specified, efficacious outcome in a mammal (for example, and not limitation, a human). Those of ordinary skill in the art will understand and appreciate the techniques appropriate for determining whether an active ingredient has a desired efficacious outcome based upon the needs of the artisan.

POLYNUCLEOTIDES shall mean RNA, DNA, or RNA/DNA hybrid sequences of more than one nucleotide in either single chain or duplex form. The polynucleotides of the invention may be prepared by any known method, including synthetic, recombinant, ex vivo generation, or a combination thereof, as well as utilizing any purification methods known in the art.

POLYPEPTIDE shall refer to a polymer of amino acids without regard to the length of the polymer. Thus, PEPTIDES, oligopeptides, and proteins are included within the definition of polypeptide. This term also does not specify or exclude post-expression modifications of polypeptides. For example, polypeptides that include the covalent attachment of glycosyl groups, acetyl groups, phosphate groups, lipid groups and the like are expressly encompassed by the term POLYPEPTIDE.

PRIMER is used herein to denote a specific oligonucleotide sequence which is complementary to a target nucleotide sequence and used to hybridize to the target nucleotide sequence. A primer serves as an initiation point for nucleotide polymerization catalyzed by DNA polymerase, RNA polymerase, or reverse transcriptase.

PURIFIED is used herein to describe a polynucleotide or polynucleotide vector of the invention that has been separated from other compounds including, but not limited to, other nucleic acids, carbohydrates, lipids and proteins (such as the enzymes used in the synthesis of the polynucleotide). In certain embodiments, a polynucleotide is substantially pure when at least about 50%, at least about 60%, at least about 75%, at least about 85%, at least about 90%, at least about 95%, at least about 96%, at least about 97%, at least about 98%, at least about 99%, or at least about 99.5% of a sample contains a single polynucleotide sequence. In some embodiments, a substantially pure polynucleotide typically comprises about 50%, about 60%, about 70%, about 80%, about 90%, about 95%, about 96%, about 97%, about 98%, about 99% or about 99.5% weight/weight of a polynucleotide sample.

Similarly, the term PURIFIED is used herein to describe a polypeptide of the invention that has been separated from other compounds including, but not limited to, nucleic acids, lipids, carbohydrates and other proteins. In certain embodiments, a polypeptide is substantially pure when at least about 50%, at least about 60%, at least about 75%, at least about 85%, at least about 90%, at least about 95%, at least about 96%, at least about 97%, at least about 98%, at least about 99%, or at least about 99.5% of the polypeptide molecules of a sample have a single amino acid sequence. In some embodiments, a substantially pure polypeptide typically comprises about 50%, about 60%, about 70%, about 80%, about 90%, about 95%, about 96%, about 97%, about 98%, about 99% or about 99.5% weight/weight of a protein sample.

Similarly, the term PURIFIED is used herein to describe a modulator of the invention. In certain embodiments, a substantially pure modulator typically comprises at least about 70%, at least about 80%, at least about 90%, at least about 95%, at least about 96%, at least about 97%, at least about 98%, at least about 99% or at least about 99.5% weight/weight of a preparation of said modulator. In certain embodiments, the modulator has an “least” purity ranging from any number, to the thousandth position, between 90% and 100% (e.g., at least 99.995% pure).

Further, as used herein, the term PURIFIED does not require absolute purity; rather, it is intended as a relative definition.

RECEPTOR FUNCTIONALITY shall refer to the normal operation of a receptor to receive a stimulus and moderate an effect in the cell, including, but not limited to regulating gene transipton, regulating the influx or efflux of ions, effecting a catalytic reaction, and/or modulating activity through G-proteins.

SECOND MESSENGER shall mean an intracellular response produced as a result of receptor activation. A second messenger can include, for example, inositol triphosphate (IP₃), diacylglycerol (DAG), cyclic AMP (cAMP), cyclic GMP (cGMP), MAP kinase acitivity, and Ca²⁺. Second messenger response can be measured for a determination of receptor activation. In addition, second messenger response can be measured for the identification of candidate compounds as, for example, inverse agonists, partial agonists, agonists, and antagonists.

SIGNAL TO NOISE RATIO shall mean the signal generated in response to activation, amplification, or stimulation wherein the signal is above the background noise or the basal level in response to non-activation, non-amplification, or non-stimulation.

SPACER shall mean a translated number of amino acids that are located after the last codon or last amino acid of a gene, for example a GPCR of interest, but before the start codon or beginning regions of the G protein of interest, wherein the translated number amino acids are placed in-frame with the beginnings regions of the G protein of interest. The number of translated amino acids can be one, two, three, four, etc., and up to twelve.

STIMULATE or STIMULATING, in relationship to the term “response” shall mean that a response is increased in the presence of a compound as opposed to in the absence of the compound.

SUBJECT shall mean primates, including but not limited to humans and baboons, as well as pet animals such as dogs and cats, laboratory animals such as rats and mice, and farm animals such as horses, sheep, and cows.

THERAPEUTICALLY EFFECTIVE AMOUNT as used herein refers to the amount of active compound or pharmaceutical agent that elicits the biological or medicinal response in a tissue, system, animal, individual or human that is being sought by a researcher, veterinarian, medical doctor or other clinician, which includes one or more of the following:

(1) Preventing the disease; for example, preventing a disease, condition or disorder in an individual that may be predisposed to the disease, condition or disorder but does not yet experience or display the pathology or symptomatology of the disease,

(2) Inhibiting the disease; for example, inhibiting a disease, condition or disorder in an individual that is experiencing or displaying the pathology or symptomatology of the disease, condition or disorder (i.e., arresting further development of the pathology and/or symptomatology), and

(3) Ameliorating the disease; for example, ameliorating a disease, condition or disorder in an individual that is experiencing or displaying the pathology or symptomatology of the disease, condition or disorder (i.e., reversing the pathology and/or symptomatology).

VARIANT as the term is used herein, is a polynucleotide or polypeptide that differs from a reference polynucleotide or polypeptide respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. A typical variant of a polypeptide differs in amino acid sequence from another, reference polypeptide. A variant and reference polypeptide may differ in amino acid sequence by one or more substitutions, additions, deletions in any combination. A variant of a polynucleotide or polypeptide may be a naturally occurring one such as an ALLELIC VARIANT, or it may be a variant that is not known to occur naturally. Non-naturally occurring variants of polynucleotides and polypeptides may be made by mutagenesis techniques or by direct synthesis.

Introduction

The order of the following sections is set forth for presentational efficiency and is not intended, nor should be construed, as a limitation on the disclosure or the claims to follow.

B. Receptor Expression
1. GPCR Polypeptides of Interest

A RUP43 GPCR of the invention comprises a GPR131 amino acid sequence. As used herein, “a GPR131 amino acid sequence” is intended to encompass the endogenous human GPR131 amino acid sequence of SEQ ID NO:2 as well as a variant amino acid sequence at least about 75%, at least about 80%, at least about 85%, at least about 90%, at least about 91%/o, at least about 92%, at least about 93%, at least about 94%, at least about 95%, at least about 96%, at least about 97%/o, at least about 98% or at least about 99% identical to the amino acid sequence of SEQ ID NO:2. In other words, a GPCR comprising a variant of the amino acid sequence of SEQ ID NO:2 also may be used in the subject methods. In certain embodiments, a GPCR that may be used in the subject methods may comprise an allelic varant of the amino acid sequence of SEQ ID NO:2. In certain embodiments, an allelic variant of the amino acid sequence of SEQ ID NO:2 is encoded by an endogenous GPR131 nucleotide sequence obtainable by performing polymerase chain reaction (PCR) on a human DNA sample using specific primers SEQ ID NO:3 and SEQ ID NO:4. In some embodiments, an allelic variant of the amino acid sequence of SEQ ID NO:2 is encoded by an endogenous GPR131 nucleotide sequence obtainable by performing polymerase chain reaction (PCR) on a human DNA sample using a specific primer comprising SEQ ID NO:3 and a specific primer comprising SEQ ID NO:4. In certain embodiments, the human DNA sample is human genomic DNA. In certain embodiments, the process is RT-PCR (reverse transcription-polymerase chain reaction). RT-PCR techniques are well known to the skilled artisan. In certain embodiments, the human cDNA sample is human monocyte or macrophage cDNA. In certain embodiments, the human cDNA sample is human adipocyte cDNA. In certain embodiments, the human cDNA sample is human skeletal muscle cell cDNA. In certain embodiments, the human DNA sample is provided. In certain embodiments, the human DNA sample is obtained from a commercial source. In certain embodiments, a variant amino acid sequence that may be used in the subject methods is a mammalian ortholog of the amino acid sequence of SEQ ID NO:2. By way of illustration and not limitation, the GPR131 amino acid sequences of rabbit (GenBank® Accession No. BAC55237, e.g.), cow (GenBank® Accession No. NP_{—}778219, e.g.), mouse (GenBank® Accession No. NP_{—}778150, e.g.), and rat (GenBank® Accession No. NP_{—}808797, e.g.) are envisioned to be within the scope of “a GPR131 amino acid sequence”. It is understood that as used herein “GPR131 GPCR” is endogenous RUP43 GPCR; by way of illustration and not limitation, endogenous human RUP43 GPCR is human GPR131 of GenBank® Accession No. NM_{—}170699 (having an amino acid sequence identical to SEQ ID NO:2) and alleles thereof, endogenous rabbit RUP43 GPCR is rabbit GPR131 of GenBank® Accession No. BAC55237 and alleles thereof, endogenous cow RUP43 GPCR is cow GPR131 of GenBank® Accession No. NP_{—}778219 and alleles thereof, endogenous mouse RUP43 GPCR is mouse GPR131 of GenBank® Accession No. NP_{—}778150 and alleles thereof, and endogenous rat RUP43 GPCR is rat GPR131 of GenBank® Accession No. NP_{—}808797 and alleles thereof.

In certain embodiments, a GPCR that may be used in the subject methods may comprise a non-endogenous, constitutively activated mutant of the amino acid sequence of SEQ ID NO:2, an allele of SEQ ID NO:2, or a mammalian ortholog of SEQ ID NO:2. As is known in the art, a constitutively activated GPCR may be made using a variety of methods (see, e.g., PCT Application Number PCT/US98/07496 published as WO 98/46995 on 22 Oct. 1998; and U.S. Pat. No. 6,555,339; the disclosure of each of which is hereby incorporated by reference in its entirety.) A biologically active fragment of the amino acid sequence of SEQ ID NO:2, of an allele of SEQ ID NO:2, of a mammalian ortholog of SEQ ID NO:2, of a non-endogenous, constitutively activated mutant of endogenous GPR131, or of an amino acid sequence at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% identical to the amino acid sequence of SEQ ID NO:2 may be used in the subject invention. By way of illustration and not limitation, deletion of an N-terminal methionine or an N-teeeil signal peptide is envisioned to provide a biologically active fragment that may be used in the subject methods. By way of further illustration and not limitation, a RUP43 GPCR that may be used in the subject methods may comprise amino acids 2-330 of SEQ ID NO:2, with the proviso that the RUP43 G protein-coupled receptor does not comprise the methionine residue at amino acid position 1 of SEQ ID NO:2;

In certain embodiments, a GPCR that may be used in the subject methods may comprise an amino acid sequence at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% identical to the amino acid sequence of SEQ ID NO:2. In certain embodiments, a GPCR that may be used in the subject methods may comprise an amino acid sequence at least about 95%, at least about 96%, at least about 97%, at least about 98% or at least about 99% identical to the amino acid sequence of SEQ ID NO:2. In certain embodiments, a GPCR that may be used in the subject methods may comprise an amino acid sequence at least about 75% identical to the amino acid sequence of SEQ ID NO:2. In certain embodiments, a GPCR that may be used in the subject methods may comprise an amino acid sequence at least about 80% identical to the amino acid sequence of SEQ ID NO:2. In certain embodiments, a GPCR that may be used in the subject methods may comprise an amino acid sequence at least about 85% identical to the amino acid sequence of SEQ ID NO:2. In certain embodiments, a GPCR that may be used in the subject methods may comprise an amino acid sequence at least about 90% identical to the amino acid sequence of SEQ ID NO:2. In certain embodiments, a GPCR that may be used in the subject methods may comprise an amino acid sequence at least about 91% identical to the amino acid sequence of SEQ ID NO:2. In certain embodiments, a GPCR that may be used in the subject methods may comprise an amino acid sequence at least about 92% identical to the amino acid sequence of SEQ ID NO:2. In certain embodiments, a GPCR that may be used in the subject methods may comprise an amino acid sequence at least about 93% identical to the amino acid sequence of SEQ ID NO:2. In certain embodiments, a GPCR that may be used in the subject methods may comprise an amino acid sequence at least about 94% identical to the amino acid sequence of SEQ ID NO:2. In certain embodiments, a GPCR that may be used in the subject methods may comprise an amino acid sequence at least about 95% identical to the amino acid sequence of SEQ ID NO:2. In certain embodiments, a GPCR that may be used in the subject methods may comprise an amino acid sequence at least about 96% identical to the amino acid sequence of SEQ ID NO:2. In certain embodiments, a GPCR that may be used in the subject methods may comprise an amino acid sequence at least about 97% identical to the amino acid sequence of SEQ ID NO:2. In certain embodiments, a GPCR that may be used in the subject methods may comprise an amino acid sequence at least about 98% identical to the amino acid sequence of SEQ ID NO:2. In certain embodiments, a GPCR that may be used in the subject methods may comprise an amino acid sequence at least about 99% identical to the amino acid sequence of SEQ ID NO:2. By an amino acid sequence having at least, for example, 95% “identity” to the amino acid sequence of SEQ ID NO:2 is meant that the amino acid sequence is identical to the amino acid sequence of SEQ ID NO:2 except that it may include up to five amino acid alterations per each 100 amino acids of the amino acid sequence of SEQ ID NO:2. Thus, to obtain an amino acid sequence having at least 95% identity to that of SEQ ID NO:2, up to 5% (5 of 100) of the amino acid residues in the sequence may be inserted, deleted, or substituted with another amino acid compared with the amino acid sequence of SEQ ID NO:2. These alternations may occur at the amino or carboxy termini or anywhere between those terminal positions, interspersed either individually among residues in the sequence or in one or more contiguous groups within the sequence.

In some embodiments, a GPR131 amino acid sequence that may be used in the subject methods is the amino acid sequence of a G protein-coupled receptor encoded by a complementary sequence to the sequence of a polynucleotide that hybridizes under stringent conditions to filter-bound DNA having the sequence set forth in SEQ ID NO:1. By way of illustration and not limitation, a GPR131 amino acid sequence that may be used in the subject methods is the amino acid sequence of a G protein-coupled receptor encoded by a polynucleotide that hybridizes under stringent conditions to the complement of SEQ ID NO:1. Hybridization techniques are well known to the skilled artisan. Preferred stringent hybridization conditions include overnight incubation at 42° C. in a solution comprising 50% formmade, 5×SSC (150 mM NaCl, 15 mM trisodium citrate), 50 mM sodium phosphate (pH 7.6), 5× Denhardt's solution, 10% dextran sulfate, and 20 μg/ml denatured, sheared salmon sperm DNA; followed by washing the filter in 0.1×SSC at about 65° C.

a. Sequence Identity

A preferred method for determining the best overall match between a query sequence (e.g., the amino acid sequence of SEQ ID NO:2) and a sequence to be interrogated, also referred to as a global sequence alignment, can be determined using the FASTDB computer program based on the algorithm of Brutlag et al. [Comp App Biosci (1990) 6:237-245; the disclosure of which is hereby incorporated by reference in its entirety]. In a sequence alignment the query and interrogated sequences are both amino acid sequences. The results of said global sequence alignment is in percent identity. Preferred parameters used in a FASTDB amino acid alignment are: Matrix=PAM 0, k-tuple=2, Mismatch Penalty=1, Joining Penalty=20, Randomization Group=25, Length=0, Cutoff Score=1, Window Size=sequence length, Gap Penalty=5, Gap Size Penalty=0.05, Window Size=247 or the length of the interrogated amino acid sequence, whichever is shorter.

If the interrogated sequence is shorter than the query sequence due to N— or C-terminal deletions, not because of internal deletions, the results, in percent identity, must be manually corrected because the FASTDB program does not account for N— and C-terminal truncations of the interrogated sequence when calculating global percent identity: For interrogated sequences truncated at the N— and C-termini, relative to the query sequence, the percent identity is corrected by calculating the number of residues of the query sequence that are N— and C-terminal of the interrogated sequence, that are not matched/aligned with a corresponding interrogated sequence residue, as a percent of the total bses of the query sequence. Whether a residue is matched/aligned is determined by results of the FASTDB sequence alignment. This percentage is then subtracted from the perecent identity, calculated by the above FASTDB program using the specified parameters, to arrive at a final percent identity score. This final percent identity score is what is used for the purposes of the present invention. Only residues to the N— and C-termini of the interrogated sequence, which are not matched/aligned with the query sequence, are considered for the purposes of manually adjusting the percent identity score. That is, only querey amino acid residues outside the farthest N— and C-terminal residues of the interrogated sequence.

For example, a 90 amino acid residue interrogated sequence is aligned with a 100-residue query sequence to determine percent identity. The deletion occurs at the N-terminus of the interrogated sequence and therefore, the FASTDB alignment does not match/align with the first residues at the N-terminus. The 10 unpaired residues represent 10% of the sequence (number of residues at the N— and C-termini not matched/total number of residues in the query sequence) so 10% is subtracted from the percent identity score calculated by the FASTDB program. If the remaining 90 residues were perfectly matched, the final percent identity would be 90%.

In another example, a 90-residue interrogated sequence is compared with a 100-residue query sequence. This time the deletions are internal so there are no residues at the N— or C-termini of the interrogated sequence, which are not matched/aligned with the query. In this case, the percent identity calculated by FASTDB is not manually corrected. Once again, only residue positions outside the N— and C-terminal ends of the subject sequence, as displayed in the FASTDB alignment, which are not matched/aligned with the query sequence are manually corrected. No other corrections are made for the purposes of the present invention.

b. Fusion Proteins

In certain embodiments, a polypeptide of interest is a fusion protein, and may contain, for example, an affinity tag domain or a reporter domain. Suitable affinity tags include any amino acid sequence that may be specifically bound to another moiety, usually another polypeptide, most usually an antibody. Suitable affinity tags include epitope tags, for example, the the V5 tag, the FLAG tag, the HA tag (from hemagglutinin influenza virus), the myc tag, and the like, as is known in the art. Suitable affinity tags also include domains for which, binding substrates are known, e.g., HIS, GST and MBP tags, as is known in the art, and domains from other proteins for which specific binding partners, e.g., antibodies, particularly monoclonal antibodies, are available. Suitable affinity tags also include any protein-protein interaction domain, such as a IgG Fc region, which may be specifically bound and detected using a suitable binding partner, e.g. the IgG Fc receptor. It is expressly contemplated that such a fusion protein may contain a heterologous N-terminal domain (e.g., an epitope tag) fused in-frame with a GPCR that has had its N-terminal methionine residue either deleted or substituted with an alternative amino acid.

Suitable reporter domains include any domain that can report the presence of a polypeptide. While it is recognized that an affinity tag may be used to report the presence of a polypeptide using, e.g., a labeled antibody that specifically binds to the tag, light emitting reporter domains are more usually used. Suitable light emitting reporter domains include luciferase (from e.g., firefly, Vargula Renilla reniformis or Renilla muelleri), or light emitting variants thereof. Other suitable reporter domains include fluorescent proteins, (from e.g., jellyfish, corals and other coelenterates as such those from Aequoria, Renilla, Ptilosarcus, Stylatula species), or light emitting variants thereof. Light emitting variants of these reporter proteins are very well known in the art and may be brighter, dimmer, or have different excitation and/or emission spectra, as compared to a native reporter protein. For example, some variants are altered such that they no longer appear green, and may appear blue, cyan, yellow, enhanced yellow red (termed BFP, CFP, YFP eYFP and RFP, respectively) or have other emission spectra, as is known in the art. Other suitable reporter domains include domains that can report the presence of a polypeptide through a biochemical or color change, such as β-galactosidase, β-glucuronidase, chloramphenicol acetyl transferase, and secreted embryonic alkaline phosphatase.

Also as is known in the art, an affinity tags or a reporter domain may be present at any position in a polypeptide of interest. However, in most embodiments, they are present at the C— or N-terminal end of a polypeptide of interest

2. Nucleic Acids Encoding GPCR Polypeptides of Interest

Since the genetic code and recombinant techniques for manipulating nucleic acid are known, and the amino acid sequences of GPCR polypeptides of interest described as above, the design and production of nucleic acids encoding a GPCR polypeptide of interest is well within the skill of an artisan. In certain embodiments, standard recombinant DNA technology (Ausubel, et al, Short Protocols in Molecular Biology, 3rd ed., Wiley & Sons, 1995; Sambrook, et al., Molecular Cloning: A Laboratory Manual, Second Edition, (1989) Cold Spring Harbor, N.Y.) methods are used. For example, GPCR coding sequences may be isolated from a library of GPCR coding sequence using any one or a combination of a variety of recombinant methods that do not need to be described herein. Subsequent substitution, deletion, and/or addition of nucleotides in the nucleic acid sequence encoding a protein may also be done using standard recombinant DNA techniques.

For example, site directed mutagenesis and subcloning may be used to introduce/delete/substitute nucleic acid residues in a polynucleotide encoding a polypeptide of interest. In other embodiments, PCR may be used. Nucleic acids encoding a polypeptide of interest may also be made by chemical synthesis entirely from oligonucleotides (e.g., Cello et al., Science (2002) 297:1016-8).

In some embodiments, the codons of the nucleic acids encoding polypeptides of interest are optimized for expression in cells of a particular species, particularly a mammalian, e.g., mouse, rat, hamster, non-human primate, or human, species. In some embodiments, the codons of the nucleic acids encoding polypeptides of interest are optimized for expression in cells of a particular species, particularly an amphibian species.

a. Vectors

The invention further provides vectors (also referred to as “constructs”) comprising a subject nucleic acid. In many embodiments of the invention, the subject nucleic acid sequences will be expressed in a host after the sequences have been operably linked to an expression control sequence, including, e.g. a promoter. The subject nucleic acids are also typically placed in an expression vector that can replicate in a host cell either as an episome or as an integral part of the host chromosomal DNA. Commonly, expression vectors will contain selection markers, e.g., tetracycline or neomycin, to permit detection of those cells transformed with the desired DNA sequences (see, e.g., U.S. Pat No. 4,704,362, which is incorporated herein by reference). Vectors, including single and dual expression cassette vectors are well known in the art (Ausubel, et al, Short Protocols in Molecular Biology, 3rd ed., Wiley & Sons, 1995; Sambrook, et al., Molecular Cloning: A Laboratory Manual, Second Edition, (1989) Cold Spring Harbor, N.Y.). Suitable vectors include viral vectors, plasmids, cosmids, artificial chromosomes (human artificial chromosomes, bacterial artificial chromosomes, yeast artificial chromosomes, etc.), mini-chromosomes, and the like. Retroviral, adenoviral and adeno-associated viral vectors may be used.

A variety of expression vectors are available to those in the art for purposes of producing a polypeptide of interest in a cell. One suitable vector is pCMV, which is used in certain embodiments. This vector was deposited with the American Type Culture Collection (ATCC) on Oct. 13, 1998 (10801 University Blvd., Manassas, Va. 20110-2209 USA) under the provisions of the Budapest Treaty for the International Recognition of the Deposit of Microorganisms for the Purpose of Patent Procedure. The DNA was tested by the ATCC and determined to be viable. The ATCC has assigned the following deposit number to pCMV: ATCC #203351.

The subject nucleic acids usually comprise an single open reading frame encoding a subject polypeptide of interest, however, in certain embodiments, since the host cell for expression of the polypeptide of interest may be a eukaryotic cell, e.g., a mammalian cell, such as a human cell, the open reading frame may be interrupted by introns. Subject nucleic acid are typically part of a transcriptional unit which may contain, in addition to the subject nucleic acid 3′ and 5′ untranslated regions (UTRs) which may direct RNA stability, translational efficiency, etc. The subject nucleic acid may also be part of an expression cassette which contains, in addition to the subject nucleic acid a promoter, which directs the transcription and expression of a polypeptide of interest, and a transcriptional terminator.

Eukaryotic promoters can be any promoter that is functional in a eukaryotic host cell, including viral promoters and promoters derived from eukaryotic genes. Exemplary eukaryotic promoters include, but are not limited to, the following: the promoter of the mouse metallothionein I gene sequence Hamer et al., J. Mol. Appl. Gen. 1:273-288, 1982); the TK promoter of Herpes virus (McKnight, Cell 31:355-365, 1982); the SV40 early promoter (Benoist et al., Nature (London) 290:304-310, 1981); the yeast gall gene sequence promoter (Johnston et al., Proc. Natl. Acad. Sci. (USA) 79:6971-6975, 1982); Silver et al., Proc. Natl. Acad. Sci. (USA) 81:5951-59SS, 1984), the CMV promoter, the EF-1 promoter, Ecdysone-responsive promoter(s), tetracycline-responsive promoter, and the like. Viral promoters may be of particular interest as they are generally particularly strong promoters. In certain embodiments, a promoter is used that is a promoter of the target pathogen. Promoters for use in the present invention are selected such that they are functional in the cell type (and/or animal) into which they are being introduced. In certain embodiments, the promoter is a CMV promoter.

In certain embodiments, a subject vector may also provide for expression of a selectable marker. Suitable vectors and selectable markers are well known in the art and discussed in Ausubel, et al. (Short Protocols in Molecular Biology, 3rd ed., Wiley & Sons, 1995) and Sambrook, et al, (Molecular Cloning: A Laboratory Manual, Third Edition, (2001) Cold Spring Harbor, N.Y.). A variety of different genes have been employed as selectable markers, and the particular gene employed in the subject vectors as a selectable marker is chosen primarily as a matter of convenience. Known selectable marker genes include: the thymidine kinase gene, the dihydrofolate reductase gene, the xanthine-guanine phosphoribosyl transferase gene, CAD, the adenosine deaminase gene, the asparagine synthetase gene, the antibiotic resistance genes, e.g. tetr, ampr, Cmr or cat, kanr or neor (aminoglycoside phosphotransferase genes), the hygromycin B phosphotransferase gene, and the like.

As mentioned above, polypeptides of interest may be fusion proteins that contain an affinity domain and/or a reporter domain. Methods for making fusions between a reporter or tag and a GPCR, for example, at the C— or N-terminus of the GPCR, are well within the skill of one of skill in the art (e.g. McLean et al, Mol. Pharma. Mol Pharmacol. 1999 56:1182-91; Ramsay et al., Br. J. Pharmacology, 2001, 315-323) and will not be described any further. It is expressly contemplated that such a fusion protein may contain a heterologous N-terminal domain (e.g., an epitope tag) fused in-frame with a GPCR that has had its N-terminal methionine residue either deleted or substituted with an alternative amino acid. It is appreciated that a polypeptide of interest may first be made from a native polypeptide and then operably linked to a suitable reporter/tag as described above.

The subject nucleic acids may also contain restriction sites, multiple cloning sites, primer binding sites, ligatable ends, recombination sites etc., usually in order to facilitate the construction of a nucleic acid encoding a polypeptide of interest.

b. Host Cells

The invention further provides host cells comprising a vector comprising a subject nucleic acid. Suitable host cells include prokaryotic, e.g., bacterial cells (for example E. coli), as well as eukaryotic cells e.g. an animal cell (for example an insect, mammal, fish, amphibian, bird or reptile cell), a plant cell (for example a maize or Arabidopsis cell), or a fungal cell (for example a S. cerevisiae cell). In certain embodiments, any cell suitable for expression of a polypeptide of interest-encoding nucleic acid may be used as a host cell. Usually, an animal host cell line is used, examples of which are as follows: monkey kidney cells (COS cells), monkey kidney CVI cells transformed by SV40 (COS-7, ATCC CRL 165 1); human embryonic kidney cells (HEK-293 [293], Graham et al. J. Gen Virol. 36:59 (1977)); HEK-293T [“293T”] cells; baby hamster kidney cells (BHK, ATCC CCL 10); chinese hamster ovary-cells (CHO, Urlaub and Chasin, Proc. Natl. Acad Sci. (USA) 77:4216, (1980); Syrian golden hamster cells MCB3901 (ATCC CRL-9595); mouse sertoli cells (TM4, Mather, Biol. Reprod. 23:243-251 (1980)); monkey kidney cells (CVI ATCC CCL 70); african green monkey kidney cells (VERO-76, ATCC CRL-1587); human cervical carcinoma cells (HELA, ATCC CCL 2), canine kidney cells (MDCK, ATCC CCL 34); buffalo rat liver cells (BRL 3A, ATCC CRL 1442); human lung cells (W138, ATCC CCL 75); human liver cells (hep G2, HB 8065); mouse mammary tumor (MMT 060562, ATCC CCL 51); TRI cells (Mather et al., Annals N.Y. Acad. Sci 383:44-68 (1982)); NIH/3T3 cells (ATCC CRL-1658); and mouse L cells (ATCC CCL-1). In certain embodiments, melanophores are used. Melanophores are skin cells found in lower vertebrates. Relevant materials and methods will be followed according to the disclosure of U.S. Pat. No. 5,462,856 and U.S. Pat. No. 6,051,386. These patent disclosures are hereby incorporated by reference in their entirety. Additional cell lines will become apparent to those of ordinary skill in the art and a wide variety of cell lines are available from the American Type Culture Collection, 10801 University Boulevard, Manassas, Va. 20110-2209.

C. Screening of Candidate Compounds
1. Generic GPCR Screening Assay Techniques

When a G protein receptor becomes active, it binds to a G protein (e.g., Gq, Gs, Gi, Gz, Go) and stimulates the binding of GTP to the G protein. The G protein then acts as a GTPase and slowly hydrolyzes the GTP to GDP, whereby the receptor, under normal conditions, becomes deactivated. However, activated receptors continue to exchange GDP to GTP. A non-hydrolyzable analog of GTP, [³⁵S]GTPγS, can be used to monitor enhanced binding to membranes which express activated receptors. It is reported that [³⁵S]GTPγS can be used to monitor G protein coupling to membranes in the absence and presence of ligand An example of this monitoring, among other examples well-known and available to those in the art, was reported by Traynor and Nahorski in 1995. A preferred use of this assay system is for initial screening of candidate compounds because the system is generically applicable to all G protein-coupled receptors regardless of the particular G protein that interacts with the intracellular domain of the receptor.
2. Specific GPCR Screening Assay Techniques

Once candidate compounds are identified using the “generic” G protein-coupled receptor assay (i.e., an assay to select compounds that are agonists or inverse agonists), in some embodiments further screening to confirm that the compounds have interacted at the receptor site is preferred. For example, a compound identified by the “generic” assay may not bind to the receptor, but may instead merely “uncouple” the G protein from the intracellular domain.

Gs, Gz and Gi.

Gs stimulates the enzyme adenylyl cyclase. Gi (and Gz and Go), on the other hand, inhibit adenylyl cyclase. Adenylyl cyclase catalyzes the conversion of ATP to cAMP; thus, activated GPCRs that couple the Gs protein are associated with increased cellular levels of cAMP. On the other hand, activated GPCRs that couple Gi (or Gz, Go) protein are associated with decreased cellular levels of cAMP. See, generally, “Indirect Mechanisms of Synaptic Transmission,” Chpt 8, From Neuron To Brain (3^{rd} Ed.) Nichols, J. G. et al eds. Sinauer Associates, Inc. (1992). Thus, assays that detect cAMP can be utilized to determine if a candidate compound is, e.g., an inverse agonist to the receptor (i.e., such a compound would decrease the levels of cAMP). A variety of approaches known in the art for measuring cAMP can be utilized, in some embodiments a preferred approach relies upon the use of anti-cAMP antibodies in an ELISA-based format. Another type of assay that can be utilized is a whole cell second messenger reporter system assay. Promoters on genes drive the expression of the proteins that a particular gene encodes. Cyclic AMP drives gene expression by promoting the binding of a cAMP-responsive DNA binding protein or transcription factor (CREB) that then binds to the promoter at specific sites called cAMP response elements and drives the expression of the gene. Reporter systems can be constructed which have a promoter containing multiple cAMP response elements before the reporter gene, e.g., β-galactosidase or luciferase. Thus, an activated Gs-linked receptor causes the accumulation of cAMP that then activates the gene and expression of the reporter protein. The reporter protein such as β-galactosidase or luciferase can then be detected using standard biochemical assays (Chen et al. 1995).

Go and Gq.

Gq and Go are associated with activation of the enzyme phospholipase C, which in turn hydrolyzes the phospholipid PIP₂, releasing two intracellular messengers: diacyclglycerol (DAG) and inositol 1,4,5-triphosphate (IP₃). Increased accumulation of IP₃ is associated with activation of Gq- and Go-associated receptors. See, generally, “Indirect Mechanisms of Synaptic Transmission,” Chpt 8, From Neuron To Brain (3^{rd} Ed.) Nichols, J. G. et al eds. Sinauer Associates, Inc. (1992). Assays that detect IP₃ accumulation can be utilized to determine if a candidate compound is, e.g., an inverse agonist to a Gq- or Go-associated receptor (i.e., such a compound would decrease the levels of IP₃). Gq-associated receptors can also been examined using an AP1 reporter assay in that Gq-dependent phospholipase C causes activation of genes containing AP1 elements; thus, activated Gq-associated receptors will evidence an increase in the expression of such genes, whereby inverse agonists thereto will evidence a decrease in such expression, and agonists will evidence an increase in such expression. Commercially available assays for such detection are available.

3. GPCR Fusion Protein

The use of an endogenous, constitutively active GPCR or a non-endogenous, constitutively activated GPCR, for use in screening of candidate compounds for the direct identification of inverse agonists or agonists provides an interesting screening challenge in that, by definition, the receptor is active even in the absence of an endogenous ligand bound thereto. Thus, in order to differentiate between, e.g., the non-endogenous receptor in the presence of a candidate compound and the non-endogenous receptor in the absence of that compound, with an aim of such a differentiation to allow for an understanding as to whether such compound may be an inverse agonist or agonist or have no affect on such a receptor, in some embodiments it is preferred that an approach be utilized that can enhance such differentiation. In some embodiments, a preferred approach is the use of a GPCR Fusion Protein

Generally, once it is determined that a non-endogenous GPCR has been constitutively activated using the assay techniques set forth above (as well as others known to the art-skilled), it is possible to determine the predominant G protein that couples with the endogenous GPCR Coupling of the G protein to the GPCR provides a signaling pathway that can be assessed. In some embodiments it is preferred that screening take place using a mammalian expression system, as such a system will be expected to have endogenous G protein therein. Thus, by definition, in such a system, the non-endogenous, constitutively activated GPCR will continuously signal. In some embodiments it is preferred that this signal be enhanced such that in the presence of, e.g., an inverse agonist to the receptor, it is more likely that it will be able to more readily differentiate, particularly in the context of screening, between the receptor when it is contacted with the inverse agonist

The GPCR Fusion Protein is intended to enhance the efficacy of G protein coupling with the non-endogenous GPCR. The GPCR Fusion Protein may be preferred for screening with either an endogenous, constitutively active GPCR or a non-endogenous, constitutively activated GPCR because such an approach increases the signal that is generated in such screening techniques. This is important in facilitating a significant “signal to noise” ratio; such a significant ratio is preferred for the screening of candidate compounds as disclosed herein.

The construction of a construct useful for expression of a GPCR Fusion Protein is within the purview of those having ordinary skill in the art. Commercially available expression vectors and systems offer a variety of approaches that can fit the particular needs of an investigator. Important criteria in the construction of such a GPCR Fusion Protein construct include but are not limited to, that the GPCR sequence and the G protein sequence both be in-frame (preferably, the sequence for the endogenous GPCR is upstream of the G protein sequence), and that the “stop” codon of the GPCR be deleted or replaced such that upon expression of the GPCR, the G protein can also be expressed. The GPCR can be linked directly to the G protein, or there can be spacer residues between the two (preferably, no more than about 12, although this number can be readily ascertained by one of ordinary skill in the art). Based upon convenience, it is preferred to use a spacer. In some embodiments, it is preferred that the G protein that couples to the non-endogenous GPCR will have been identified prior to the creation of the GPCR Fusion Protein construct. Because there are only a few G proteins that have been identified, it is preferred that a construct comprising the sequence of the G protein (i.e., a universal G protein construct, see Example 5(a) below) be available for insertion of an endogenous GPCR sequence therein; this provides for further efficiency in the context of large-scale screening of a variety of different endogenous GPCRs having different sequences.

As noted above, activated GPCRs that couple to Gi, Gz and Go are expected to inhibit the formation of cAMP making assays based upon these types of GPCRs challenging [i.e., the cAMP signal decreases upon activation, thus making the direct identification of, e.g., agonists (which would further decrease this signal) challenging]. As will be disclosed herein, it has been ascertained that for these types of receptors, it is possible to create a GPCR Fusion Protein that is not based upon the GPCR's endogenous G protein, in an effort to establish a viable cyclase-based assay. Thus, for example, an endogenous Gi coupled receptor can be fused to a Gs protein—such a fusion construct, upon expression, “drives” or “forces” the endogenous GPCR to couple with, e.g., Gs rather than the “natural” Gi protein, such that a cyclase-based assay can be established. Thus, for Gi, Gz and Go coupled receptors, in some embodiments it is preferred that when a GPCR Fusion Protein is used and the assay is based upon detection of adenylyl cyclase activity, that the fusion construct be established with Gs (or an equivalent G protein that stimulates the formation of the enzyme adenylyl cyclase).

TABLE B

| | Effect on cAMP | Effect on IP₃ | | |
|---|---|---|---|---|
| | Production upon | Accumulation | | |
| | Activation of | upon Activation of | | |
| | GPCR (i.e., | GPCR (i.e., | Effect on cAMP | Effect on IP₃ |
| | constitutive | constitutive | Production upon | Accumulation upon |
| | activation or | activation or | contact with an | contact with an |
| G protein | agonist binding) | agonist binding) | Inverse Agonist | Inverse Agonist |
| Gs | Increase | N/A | Decrease | N/A |
| Gi | Decrease | N/A | Increase | N/A |
| Gz | Decrease | N/A | Increase | N/A |
| Go | Decrease | Increase | Increase | Decrease |
| Gq | N/A | Increase | N/A | Decrease |

Equally effective is a G Protein Fusion construct that utilizes a Gq Protein fused with a Gs, Gi, Gz or Go Protein. In some embodiments a preferred fusion construct can be accomplished with a Gq Protein wherein the first six (6) amino acids of the G-protein α-subunit (“Gαq”) is deleted and the last five (5) amino acids at the C-terminal end of Gαq is replaced with the corresponding amino acids of the Gα of the G protein of interest. For example, a fusion construct can have a Gq (6 amino acid deletion) fused with a Gi Protein, resulting in a “Gq/Gi Fusion Construct”. This fusion construct will forces the endogenous Gi coupled receptor to couple to its non-endogenous G protein, Gq, such that the second messenger, for example, inositol triphosphate or diacylglycerol can be measured in lieu of cAMP production.

Co-transfection of a Target Gi Coupled GPCR with a Signal-Enhancer Gs Coupled GPCR (cAMP Based Assays)

A Gi coupled receptor is known to inhibit adenylyl cyclase, and, therefore, decreases the level of cAMP production, which can make the assessment of cAMP levels challenging. In certain embodiments, an effective technique in measuring the decrease in production of cAMP as an indication of activation of a receptor that predominantly couples Gi upon activation can be accomplished by co-transfecting a signal enhancer, e.g., a non-endogenous, constitutively activated receptor that predominantly couples with Gs upon activation (e.g., TSHR-A623I; see infra), with the Gi linked GPCR As is apparent, activation of a Gs coupled receptor can be determined based upon an increase in production of cAMP. Activation of a Gi coupled receptor leads to a decrease in production cAMP. Thus, the co-transfection approach is intended to advantageously exploit these “opposite” affects. For example, co-transfection of a non-endogenous, constitutively activated Gs coupled receptor (the “signal enhancer”) with expression vector alone provides a baseline cAMP signal (i.e., although the Gi coupled receptor will decrease cAMP levels, this “decrease” will be relative to the substantial increase in cAMP levels established by constitutively activated Gs coupled signal enhancer). By then transfecting the signal enhancer with the “target receptor”, an inverse agonist of the Gi coupled target receptor will increase the measured CAMP signal, while an agonist of the Gi coupled target receptor will decrease this signal.

Candidate compounds that are directly identified using this approach should be assessed independently to ensure that these do not target the signal enhancing receptor (this can be done prior to or after screening against the co-transfected receptors).

D. Medicinal Chemistry

Candidate Compounds

Any molecule known in the art can be tested for its ability to modulate (increase or decrease) the activity of a GPCR of the present invention. For identifying a compound that modulates activity, candidate compounds can be directly provided to a cell expressing the receptor.

This embodiment of the invention is well suited to screen chemical libraries for molecules which modulate, e.g., inhibit, antagonize, or agonize, the amount of, or activity of, a receptor. The chemical libraries can be peptide libraries, peptidomimetic libraries, chemically synthesized libraries, recombinant, e.g., phage display libraries, and in vitro translation-based libraries, other non-peptide synthetic organic libraries, etc. This embodiment of the invention is also well suited to screen endogenous candidate compounds comprising biological materials, including but not limited to plasma and tissue extracts, and to screen libraries of endogenous compounds known to have biological activity.

In some embodiments direct identification of candidate compounds is conducted in conjunction with compounds generated via combinatorial chemistry techniques, whereby thousands of compounds are randomly prepared for such analysis. The candidate compound may be a member of a chemical library. This may comprise any convenient number of individual members, for example tens to hundreds to thousand to millions of suitable compounds, for example peptides, peptoids and other oligomeric compounds (cyclic or linear), and template-based smaller molecules, for example benzodiazepines, hydantoins, biaryls, carbocyclic and polycyclic compounds (e.g., naphthalenes, phenothiazines, acridines, steroids etc.), carbohydrate and amino acid derivatives, dihydropyridines, benzhydryls and heterocycles (e.g., trizines, indoles, thiazolidines etc.). The numbers quoted and the types of compounds listed are illustrative, but not limiting. Preferred chemical libraries comprise chemical compounds of low molecular weight and potential therapeutic agents.

Exemplary chemical libraries are commercially available from several sources (ArQule, Tripos/PanLabs, ChemDesign, Pharmacopoeia). In some cases, these chemical libraries are generated using combinatorial strategies that encode the identity of each member of the library on a substrate to which the member compound is attached, thus allowing direct and immediate identification of a molecule that is an effective modulator. Thus, in many combinatorial approaches, the position on a plate of a compound specifies that compound's composition. Also, in one example, a single plate position may have from 1-20 chemicals that can be screened by administration to a well containing the interactions of interest. Thus, if modulation is detected, smaller and smaller pools of interacting pairs can be assayed for the modulation activity. By such methods, many candidate molecules can be screened.

Many diversity libraries suitable for use are known in the art and can be used to provide compounds to be tested according to the present invention. Alternatively, libraries can be constructed using standard methods. Further, more general, structurally constrained, organic diversity (e.g., nonpeptide) libraries, can also be used. By way of example, a benzodiazepine library (see e.g., Bunin et al., 1994, Proc. Natl. Acad. Sci. USA 91:4708-4712) may be used.

In another embodiment of the present invention, combinatorial chemistry can be used to identify modulators of the GPCRs of the present invention. Combinatorial chemistry is capable of creating libraries containing hundreds of thousands of compounds, many of which may be structurally similar. While high throughput screening programs are capable of screening these vast libraries for affinity for known targets, new approaches have been developed that achieve libraries of smaller dimension but which provide maximum chemical diversity. (See e.g., Matter, 1997, Journal of Medicinal Chemistry 40:1219-1229).

One method of combinatorial chemistry, affinity fingerprinting, has previously been used to test a discrete library of small molecules for binding affinities for a defined panel of proteins. The fingerprints obtained by the screen are used to predict the affinity of the individual library members for other proteins or receptors of interest (in the instant invention, the receptors of the present invention). The fingerprints are compared with fingerprints obtained from other compounds known to react with the protein of interest to predict whether the library compound might similarly react. For example, rather than testing every ligand in a large library for interaction with a complex or protein component, only those ligands having a fingerprint similar to other compounds known to have that activity could be tested (See, e.g., Kauvar et al., 1995, Chemistry and Biology 2:107-118; Kauvar, 1995, Affinity fingerprinting, Pharmaceutical Manufacturing International. 8:25-28; and Kauvar, Toxic-Chemical Detection by Pattern Recognition in New Frontiers in Agrochemical Immunoassay, D. Kurtz. L. Stanker and J. H. Skerritt Editors, 1995, AOAC: Washington, D.C., 305-312).

Candidate Compounds Identified as Modulators

Generally, the results of such screening will be compounds having unique core structures; thereafter, these compounds may be subjected to additional chemical modification around a preferred core structure(s) to further enhance the medicinal properties thereof. Such techniques are known to those in the art and will not be addressed in detail in this patent document.

In certain embodiments, said identified modulator is bioavailable. A number of computational approaches available to those of ordinary skill in the art have been developed for prediction of oral bioavailability of a drug [Ooms et al., Biochim Biophys Acta (2002) 1587:118-25; Clark & Grootenhuis, Curr OpinDrug Discov Devel (2002) 5:382-90; Cheng et al., J Comput Chem (2002) 23:172-83; Norinder & Haeberlein, Adv Drug Deliv Rev (2002) 54:291-313; Matter et al., Comb Chem High Throughput Screen (2001) 4:453-75; Podlogar & Muegge, Curr Top Med Chem (2001) 1:257-75; the disclosure of each of which is hereby incorporated by reference in its entirety). Furthermore, positron emission tomography (PET) has been successfully used by a number of groups to obtain direct measurements of drug distribution, including an assessment of oral bioavailability, in the mammalian body following oral administration of the drug, including non-human primate and human body [Noda et al., J Nucl Med (2003) 44:105-8; Gulyas et al., Eur J Nucl Med Mol Imaging (2002) 29:1031-8; Kanerva et al., Psychopharmacology (1999) 145:76-81; the disclosure of each of which is hereby incorporated by reference in its entirety]. Also, see infra, including Example 25.

In certain embodiments, said bioavailable identified modulator further is able to cross the blood-brain barrier. A number of computational approaches available to those of ordinary skill in the art have been developed for prediction of the permeation of the blood-brain barrier [Ooms et al., Biochim Biophys Acta (2002) 1587:118-25; Clark & Grootenhuis, Curr OpinDrug Discov Devel (2002) 5:382-90; Cheng et al., J Comput Chem (2002) 23:172-83; Norinder & Haeberlein, Adv Drug Deliv Rev (2002) 54:291-313; Matter et al., Comb Chem High Throughput Screen (2001) 4:453-75; Podlogar & Muegge, Curr Top Med Chem (2001) 1:257-75; the disclosure of each of which is hereby incorporated by reference in its entirety). A number of in vitro methods have been developed to predict blood-brain barrier permeability of durgs [Lohmann et al., J Drug Target (2002) 10:263-76; Hansen et al., J Pharm Biomed Anal (2002) 27:945-58; Otis et al., J Pharmocol Toxicol Methods (2001) 45:71-7; Dehouck et al, J Neurochem (1990) 54:1798-801; the disclosure of each of which is hereby incorporated by reference in its entirety]. Furthermore, a number of strategies have been developed to enhance drug delivery across the blood-brain barrier [Scherrmann, Vascul Pharmacol (2002) 38:349-54; Pardridge, Arch Neurol (2002) 59:3540; Pardridge, Neuron (2002) 36:555-8; the disclosure of each of which is hereby incorporated by refrence in its entirety]. Finally, positron emission tomography (PET) has been successfully used by a number of groups to obtain direct measurements of drug distribution, including that within brain, in the mammalian body, including non-human primate and human body [Noda et al., J Nucl Med (2003) 44:105-8; Gulyas et al., Eur J Nucl Med Mol Imaging (2002) 29:1031-8; Kanerva et al., Psychopharmacology (1999) 145:76-81; the disclosure of each of which is hereby incorporated by reference in its entirety]. Also, see infra, including Example 26.

E. Compounds of the Invention

One aspect of the present invention pertains to a compound of Formula (II):

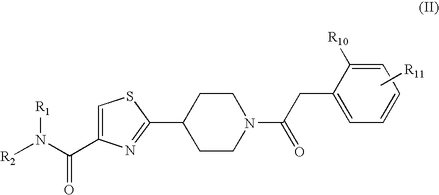

or a pharmaceutically acceptable salt thereof,

wherein:

R₁ is H or C₁₋₆ alkyl,

R₂ is a 2-methyl-4,5,6,7-tetrahydro-2H-indazol-3-yl group; or

R₁ and R₂ together with the nitrogen to which they are bonded form a 3,4-dihydro-2H-quinoline-1-yl group; and

R₁₀ and R₁₁ are each independently H or halogen.

F. Synthetic Methods for Making Compounds of the Invention

Preparation of Compounds of the Present Invention—General Synthetic Methods

The novel compounds of the present invention can be readily prepared according to a variety of synthetic methods, all of which would be familiar to one skilled in the art. Certain methods for the preparation of compounds of the present invention include, but are not limited to, those described in Schemes 1-3, infra.

The intermediate (AD) of the novel 2-piperidin-4-yl-thiazoles can be prepared as shown in Scheme 1. The thioamide (AA), protected at the nitrogen with a suitable protecting group (i.e. PG), is cyclized via a Hantzsch-like reaction with 3-halo-2-oxo-propionic acid (AB), protected at the carboxylic acid, to give di-protected 2-piperidin-4-yl-thiazole (AC). Generally the two protecting groups are different. Suitable solvents for the cyclization include, for example, alcohols (such as, methanol ethanol and propanol), lower halocarbons (such as, dichloromethane, dichloroethane and chloroform), DMF, and the like. Reaction temperatures for the cyclization can range from about room temperature to about the boiling point of the solvent used; generally the temperature range is about 50° C. to about 90° C.

Suitable protecting groups for thioamide (AA) include t-butyl carbamate (BOC), benzyl carbamate (Cbz), p-methoxybenzyl carbamate (Moz), and the like. Various methods can be used to protect the nitrogen of thioamide (AA). For example, the t-butyl carbamate group can be introduced using a variety of reagents, such as (BOC)₂O, with a suitable base (such as, NaOH, KOH, or Me₄NOH) and in a suitable solvent(s) (THF, CH₃CN, DMF, EtOH, MeOH, H₂O, or mixtures thereof) at a temperature of about 0° C. to about 50° C.

Suitable protecting groups for 3-halo-2-oxo-propionic acid (AB) include alkyl esters (such as methyl ethyl, propyl and t-butyl), substituted methyl esters (such as, methoxymethyl, methoxyethoxymethyl, and benzyloxymethyl), optionally substituted benzyl esters (such as, benzyl 4-methoxybenzyl, and 2,6dimethoxybenzyl), and the like. One particular useful protected 3-halo-2-oxo-propionic acid (AB) is 3-bromo-2-oxo-propionic acid ethyl ester, also commonly referred to as ethyl bromopyruvate.

Other representative protecting groups suitable for a wide variety of synthetic transformations are disclosed in Greene and Wuts, Protective Groups in Organic Synthesis, third edition, John Wiley & Sons, New York, 1999, the disclosure of which is incorporated herein by reference in its entirety.

For convenience, the two protecting groups in 2-piperidin-4-yl-thiazole (AC) are selected so one protecting group can be substantially removed without substantially affecting the other protecting group. This type of strategy is referred to as orthogonal protection. One example includes, protecting the nitrogen with a BOC group and protecting the carboxylic acid as a methyl or ethyl ester. In this example, the BOC group can be removed under acidic conditions without substantially affecting the ester group. Alternatively, since the BOC group is not substantially hydrolyzed under basic conditions the ester can be removed without substantially affecting the BOC group. Many orthogonal protection schemes are known in the art and can be applied herein.

Subsequently, as shown in Scheme 1, the nitrogen protecting group for 2-piperidin4-yl-thiazoles (AC) is removed (i.e. deprotected), while substantially maintaining the carboxylic acid protection, to give common intermediate (AD). In the case when the nitrogen is protected with a BOC group effective cleavage can be achieved in the presence of an acid and optionally in a suitable solvent. Suitable acids include, HCl (aqueous or anhydrous), HBr (aqueous or anhydrous), H₂SO₄, trifluoroacetic acid, p-toluenesulfonic acid, and the like. When present, suitable solvents include, ester solvents (such as, ethyl acetate), alkyl alcohols (such as, methanol, ethanol, i-propanol, n-propanol and n-butanol), ethereal solvents (such as, tetrahydrofuran and dioxane), and the like or mixtures thereof. Optionally a scavenger can be added to capture the liberated cations. Suitable scavengers include, thiophenol anisole, thioanisole, thiocresol, cresol, dimethyl sulfide and the like. Reaction temperature ranges for the deprotection of the nitrogen in 2-piperidin-4-yl-thiazoles (AC) can range from about −20° C. to about the boiling point of the solvent used; generally the temperature range is about −10° C. to about 50° C.

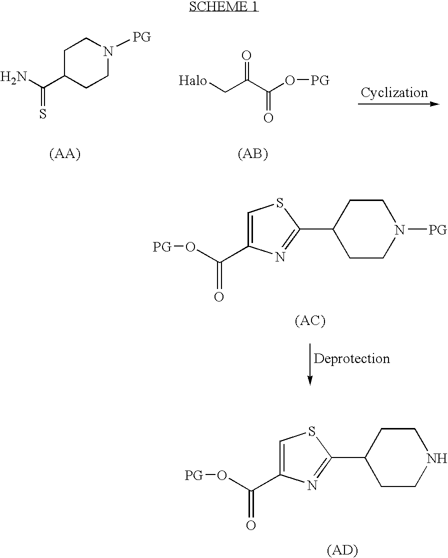

The intermediate (AD) is coupled with a carboxylic acid in the presence of a dehydrating condensing agent and an inert solvent with or without a base to provide amide (AE) as illustrated in Scheme 2, Method A. Suitable dehydrating condensing agents include dicyclohexylcarbo-diimide (DCC), 1-ethyl-3-(3-dimethylaminopropyl)carbodiimide hydrochloride (EDC.HCl), bromo-tis-pyrrolidino-phosnium hexafluorophosphate (PyBroP), O-7-azabenzotriazol-1-yl)-1,1,3,3-tetramethyluronium hexafluorophosphate (HATU), 1-cyclohexyl-3-methylpolystyrene-carbodiimide and the like. Suitable bases include tertiary amines (such as, N,N-diisopropyl-ethylamine, N-methylmorpholine, and triethylamine). Suitable inert solvents include lower halocarbon solvents (preferably dichloromethane, dichloroethane, and chloroform), ethereal solvents (such as, tetrahydrofuran and dioxane), nitrile solvents (such as acetonitrile), amide solvents (such as, N,N-dimethylformamide, and N,N-dimethylacetamide), or mixtures thereof. Optionally, other reagents can be used in the coupling reaction and these reagents include, 1-hydroxybenzotriazole (HOBT), HOBT-6-carboxaamidomethyl polystyrene, 1-hydroxy-7-azabenzotriazole (HOAT) and the like. Suitable reaction temperature ranges from about −25° C. to about 60° C., and about 0° C. to about 35° C.

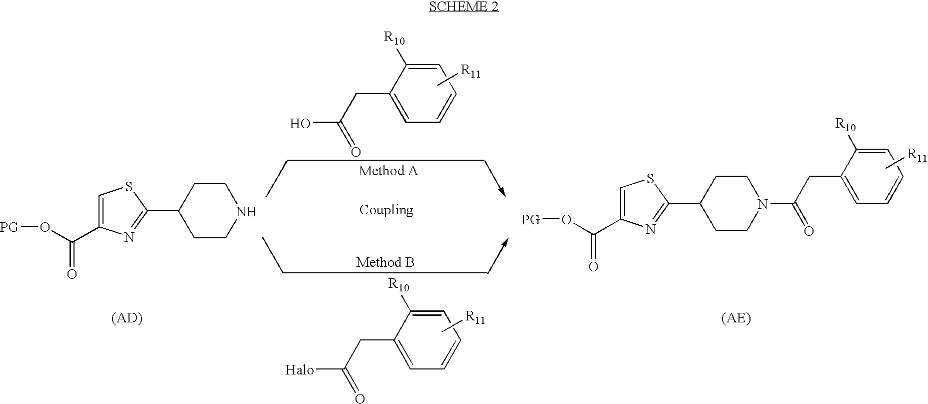

Alternatively, amide (AE) can be obtained by an amidation reaction using an acid halide with intermediate (AD) in the presence of a base and an inert solvent as shown in Scheme 2, Method B. Suitable acid halides, include, acid chlorides or acid bromides. Suitable bases include alkali metal carbonates (such as, sodium carbonate and potassium carbonate), alkali metal hydrogencarbonates (such as, sodium hydrogencarbonate and potassium hydrogencarbonate), alkali hydroxides (such as, sodium hydroxide and potassium hydroxide), tertiary amines (such as, N,N-diisopropylethylamine, triethylamine, and N-methylmorpholine), and aromatic amines (such as, pyridine, imidazole, and poly-(4-vinylpyridine)). Suitable inert solvents include lower halocarbon solvents (such as, dichloromethane, dichloroethane, and chloroform), ethereal solvents (such as, tetrahydrofuran and dioxane), amide solvents (such as, N,N-dimethylformamide, and N,N-dimethylacetamide), and aromatic solvents (such as toluene, benzene, and pyridine). Suitable reaction temperature ranges from about −25° C. to about 55° C., preferably about −5° C. to about 40° C.

The protected acid group in amide (AE) is removed to give the corresponding carboxylic acid as shown in Scheme 3. Suitable methods for deprotecting the carboxylic acid are known to those of originally skill in the art. For example, alkyl esters (such as, methyl, ethyl, and n-propyl) can be converted to carboxylic acids via hydrolysis in the presence of a base and in a suitable solvent. Suitable bases include, alkali metal carbonates (such as, sodium carbonate and potassium carbonate), alkali metal hydrogencarbonates (such as, sodium hydrogencarbonate and potassium hydrogencarbonate), and alkali hydroxides (such as, lithium hydroxide, sodium hydroxide and potassium hydroxide). Suitable solvents for the deprotection include, alkyl alcohols (such as, methanol ethanol i-propanol, n-propanol and n-butanol), ethereal solvents (such as, tetrahydrofuran and dioxane), and the like or mixture thereof, preferably the hydrolysis is conducted in the presence of H₂O. Reaction temperatures for the deprotection of the acid group in amide (AE) can range from about room temperature to about the boiling point of the solvent used; generally the temperature range is about 50° C. to about 90° C. Other deprotection methods for esters as well as other additional suitable protecting groups are described in Greene and Wuts, Protect Groups in Organic Synthesis, third edition, John Wiley & Sons, New York, 1999, supra. The carboxylic acid (AF) is coupled with either methyl-(2-methyl-4,5,6,7-tetrahydro-2H-indazol-3-yl)-amine or 1,2,3,4tetrahydro-quinoline to give compounds of Formulae (AG) and (AH) respectively. Generally the coupling can be conducted in the presence of a dehydrating condensing agent and an inert solvent with or without a base, or by an amidation reaction using an acid halide generated from carboxylic acid (AF) in the presence of a base and an inert solvent, each method is as described for Scheme 2, supra.

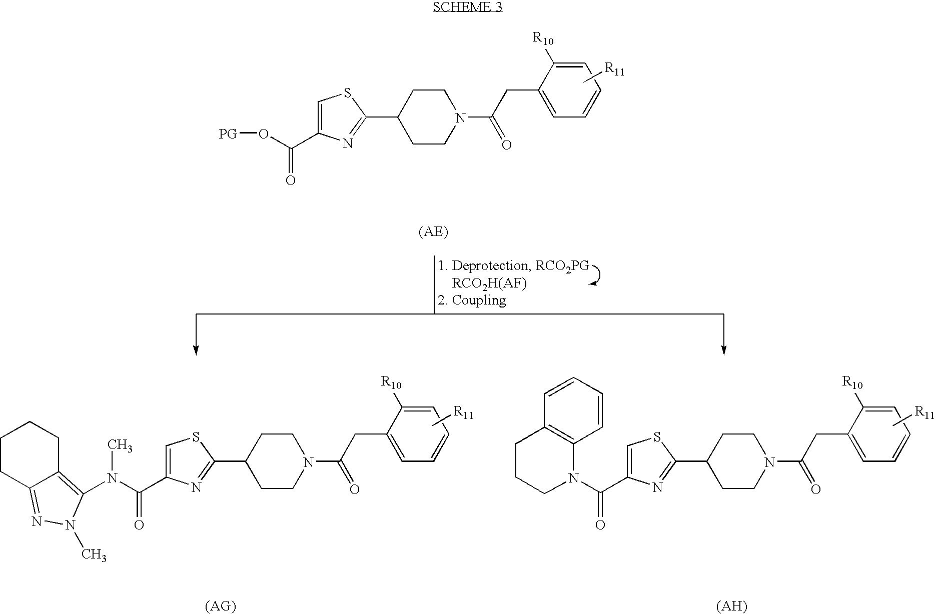

Some embodiments of the present invention include compounds illustrated in TABLE 1 as shown below.

TABLE 1

| Cmpd# | Structure | Chemical Name |
|---|---|---|
| 1 | 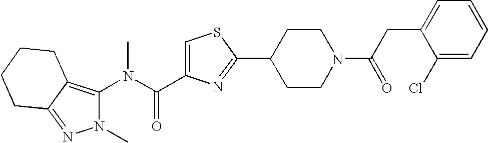 | 2-{1-[2-(2- Chloro-phenyl)-acetyl]- piperidin-4-yl}- thiazole-4-carboxylic acid methyl-(2-methyl- 4,5,6,7-tetrahydro-2H- indazol-3-yl)-amide |
| 2 | 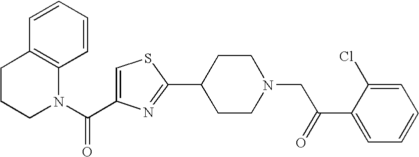 | 2-(2-Chloro- phenyl)-1-{4-[4-(3,4- dihydro-2H-quinoline- 1-carbonyl)-thiazol-2- yl]-piperidin-1-yl}- ethanone |
| 3 | 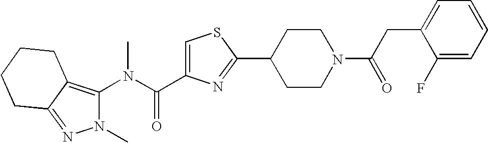 | 2-{1-[2-(2- Fluoro-phenyl)-acetyl]- piperidin-4-yl}- thiazole-4-carboxylic acid methyl-(2-methyl- 4,5,6,7-tetrahydro-2H- indazol-3-yl)-amide |

G. Pharmaceutical Compositions

The invention provides methods of treatment (and prevention) by administration to an individual in need of said treatment (or prevention) a therapeutically effect amount of a modulator of the invention [also see, e.g., PCT Application No. PCT/IB02/01461 published as WO 02/066505 on 29 Aug. 2002; the disclosure of each of which is hereby incorporated by reference in its entirety]. In a preferred aspect, the modulator is an agonist. In a preferred aspect, the modulator is substantially purified. The individual is preferably an animal including, but not limited to animals such as cows, pigs, horses, chickens, non-human primates, cats, dogs, rabbits, rats, mice, etc., and is preferably a mammal, and most preferably human.

Modulators of the invention can be administered to non-human animals [see Examples, infra] and/or humans, alone or in pharmaceutical or physiologically acceptable compositions where they are mixed with suitable carriers or excipient(s) using techniques well known to those in the art. Suitable pharmaceutically-acceptable carriers are available to those in the art; for example, see Remington's Pharmaceutical Sciences, 16^{th} Edition, 1980, Mack Publishing Co., (Oslo et al., eds.).

The pharmaceutical or physiologically acceptable composition is then provided at a therapeutically effective dose. A therapeutically effective dose refers to that amount of a modulator sufficient to result in prevention or amelioration of symptoms or physiological status of a disorder as determined illustratively and not by limitation by the methods described herein, wherein the prevention or amelioration of symptoms or physiological status of a disorder includes but is not limited to lowering of blood glucose concentration, prevention or treatment of certain metabolic disorders, such as insulin resistance, impaired glucose tolerance, and diabetes, and prevention or treatment of a complication of an elevated blood glucose concentration, such as atherosclerosis, heart disease, stroke, hypertension and peripheral vascular disease.

It is expressly considered that the modulators of the invention may be provided alone or in combination with other pharmaceutically or physiologically acceptable compounds. Other compounds for the treatment of disorders of the invention, wherein the treatment of disorders of the invention includes but is not limited to lowering of blood glucose concentration, prevention or treatment of certain metabolic disorders, such as insulin resistance, impaired glucose tolerance, and diabetes, and prevention or treatment of a complication of an elevated blood glucose concentration, such as atherosclerosis, heart disease, stroke, hypertension and peripheral vascular disease, are currently well known in the art. One aspect of the invention encompasses the use according to embodiments disclosed herein further comprising one or more agents selected from the group consisting of sulfonylurea (e.g., glibenclamide, glipizide, gliclazide, glimepiride), meglitinide (e.g., repaglinide, nateglanide), biguanide (e.g., metformin), alpha-glucosidase inhibitor (e.g., acarbose, epalrestat, miglitol, voglibose), thizaolidinedione (e.g., rosiglitazone, pioglitazone), insulin analog (e.g., insulin lispro, insulin aspart, insulin glargine), chromium picolinate/biotin, and biological agent (e.g., adiponectin or a fragment comprising the C-terminal globular domain thereof, or a multimer of adiponectin or said fragment thereof; or an agonist of adiponectin receptor AdipoR1 or AdipoR2, preferably wherein said agonist is orally bioavailable). Additionally, it is expressly contemplated that the modulators of the invention, e.g. agonists and partial agonists of the invention, may be provided alone or in combination with a phosphodiesterase (PDE) inhibitor (inclusive of an inhibitor selective for type 4 cAMP-specific PDE (PDE4), e.g. roflumilast; an inhibitor selective for PDE4B; and an inhibitor selective for PDE4B2).

In certain embodiments, the metabolic disorder is selected from the group consisting of impaired glucose tolerance, insulin resistance, hyperinsulinemia, and diabetes. In some embodiments, diabetes is type 1 diabetes. In certain preferred embodiments, diabetes is type 2 diabetes. In certain embodiments, the metabolic disorder is diabetes. In certain embodiments, the metabolic disorder is type 1 diabetes. In certain embodiments, the metabolic disorder is type 2 diabetes. In certain embodiments, the metabolic disorder is impaired glucose tolerance. In certain embodiments, the metabolic disorder is insulin resistance. In certain embodiments, the metabolic disorder is hyperinsulinemia. In certain embodiments, the metabolic disorder is related to an elevated blood glucose concentration in the individual.

In certain embodiments, the complication of an elevated blood glucose concentration is selected from the group consisting of Syndrome X, atherosclerosis, atheromatous disease, heart disease, hypertension, stroke, neuropathy, retinopathy, nephropathy, and peripheral vascular disease. Heart disease includes, but is not limited to, cardiac insufficiency, coronary insufficiency, coronary artery disease, and high blood pressure. In certain embodiments, the complication is Syndrome X In certain embodiments, the complication is atherosclerosis. In certain embodiments, the complication is atheromatous disease. In certain embodiments, the complication is heart disease. In certain embodiments, the complication is cardiac insufficiency. In certain embodiments, the complication is coronary insufficiency. In certain embodiments, the complication is coronary artery disease. In certain embodiments, the complication is high blood pressure. In certain embodiments, the complication is hypertension. In certain embodiments, the complication is stroke. In certain embodiments, the complication is neuropathy. In certain embodiments, the complication is retinopathy. In certain embodiments, the complication is neuropathy. In certain embodiments, the complication is peripheral vascular disease. In certain embodiments, the complication is polycystic ovary syndrome. In certain embodiments, the complication is hyperlipidemia.

Routes of Administration

Suitable routes of administration include oral nasal, rectal, transmucosal, transdermal or intestinal administration, parenteral delivery, including intramuscular, subcutaneous, intramedullary injections, as well as intrathecal direct intraventricular, intravenous, intraperitoneal, intranasal, intrapulmonary (inhaled) or intraocular injections using methods known in the art. Other particularly preferred routes of administration are aerosol and depot formulation. Sustained release formulations, particularly depot, of the invented medicaments are expressly contemplated. In certain embodiments, route of administration is oral.

Composition/Formulation

Pharmaceutical or physiologically acceptable compositions and medicaments for use in accordance with the present invention may be formulated in a conventional manner using one or more physiologically acceptable carriers comprising excipients and auxiliaries. Proper formulation is dependent upon the route of administration chosen.

Certain of the medicaments described herein will include a pharmaceutically or physiologically acceptable carrier and at least one modulator of the invention. For injection, the agents of the invention may be formulated in aqueous solutions, preferably in physiologically compatible buffers such as Hanks's solution, Ringer's solution, or physiological saline buffer such as a phosphate or bicarbonate buffer. For transmucosal administration, penetrants appropriate to the barrier to be permeated are used in the formulation. Such penetrants are generally known in the art.

Pharmaceutical or physiologically acceptable preparations that can be taken orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate and, optionally, stabilize. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs for a nebulizer, with the use of a suitable gaseous propellant, e.g., carbon dioxide. In the case of a pressurized aerosol the dosage unit may be determined by providing a valve to deliver a metered amount. Capsules and cartridges of, e.g., gelatin, for ue in an inhaler or insufflator, may be forumulated containing a powder mix of the compound and a suitable powder base such as lactose or starch.

The compounds may be formulated for parenteral administration by injection, e.g., by bolus injection or continuous infusion. Formations for injection may be presented in unit dosage for, e.g., in ampoules or in multi-dose containers, with an added preservative. The compositions may take such forms as suspension, solutions or emulsions in aqueous vehicles, and may contain formullatory agents such as suspending, stabilizing and/or dispersing agents.

Pharmaceutical or physiologically acceptable formulations for parenteral administration include aqueous solutions of the active compounds in water-soluble form. Aqueous suspension may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

Alternatively, the active ingredient may be in powder or lyophilized form for constitution with a suitable vehicle, such as sterile pyrogen-free water, before use.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

In a particular embodiment, the compounds can be delivered via a controlled release system. In one embodiment, a pump may be used (Langer, supra; Sefton, 1987, CRC Crit. Ref. Biomed. Eng. 14:201-240; Buchwald et al., 1980, Surgery 88:507-516; Saudek et al., 1989, N. Engl. J. Med. 321 :574-579). In another embodiment, polymeric materials can be used (Medical Applications of Controlled Release, Langer and Wise, eds., CRC Press, Boca Raton, Fla., 1974; Controlled Drug Bioavailability, Drug Product Design and Performance, Smolen and Ball, eds., Wiley, N.Y., 1984; Ranger and Peppas, 1983, Macromol. Sci. Rev. Macromol. Chem. 23:61; Levy et al., 1985, Science 228:190-192; During et al., 1989, Ann. Neurol. 25:351-356; Howard et al., 1989, J. Neurosurg. 71:858-863). Other controlled release systems are discussed in the review by Langer (1990, Science 249:1527-1533).

Additionally, the compounds may be delivered using a sustained-release system, such as semipermeable matrices of solid hydrophobic polymers containing the therapeutic agent. Various sustained release materials have been established and are well known by those skilled in the art. Sustained-release capsules may, depending on their chemical nature, release the compounds for a few weeks up to over 100 days.

Depending on the chemical nature and the biological stability of the therapeutic reagent, additional strategies for modulator stabilization may be employed.

The pharmaceutical or physiologically acceptable compositions also may comprise suitable solid or gel phase carriers or excipients. Examples of such carriers or escipients include but are not limited to calcium carbonate, calcium phosphate, various sugars, starches, cellulos derivatives, gelatin, and polymers such as polyethylene glycols.

Effective Dosage

Pharmaceutical or physiologically acceptable compositions suitable for use in the present invention include compositions wherein the active ingredients are contained in an effective amount to achieve their intended purpose. More specifically, a therapeutically effective amount means an amount effective to prevent development of or to alleviate the existing symptoms of the subject being treated. Determination of the effective amounts is wll within the capability of those skilled in the art, especially in light of the detailed disclosure provided herein.

For any compound used in the method of the invention, the therapeutically effective dose can be estimated initially from cell culture assays. For example, a dose can be formulated in animal models to achieve a circulating concentration range that includes or encompasses a concentration point or range shown to stimulate glucose uptake in a cell to prevent or treat certain metabolic disorders, or to prevent or treat a complication of elevated blood glucose concentration. [See Examples, infra, for in vitro assays and in vivo animal models.] Such information can be used to more accurately determine usefull doses in humans.

A therapeutically effective dose refers to that amount of the compound that results in amelioration of symptoms in a patient. Toxicity and therapeutic efficacy of such compounds can be determined by standard pharmaceutical procedures in cell cultures or experimental animals, e.g., for determining the LD₅₀ (the dose lethal to 50% of the test population) and the ED₅₀ (the dose therapeutically effective in 50% of the test population). The dose ratio between toxic and therapeutic effects is the therapeutic index and it can be expressed as the ratio between LD₅₀ and ED₅₀. Compounds that exhibit high therapeutic indices are preferred.

The data obtained from these cell culture assays and animal studies can be used in formulating a range of dosage for use in humans. The dosage of such compounds lies preferably within a range of circulating concentrations that include the ED50, with little or no toxicity. The dosage may vary within this range depending upon the dosage form employed and the route of administration utilized. The exact formulation, route of administration and dosage can be chosen by the individual physician in view of the patient's condition. (See, e.g., Fingl et al., 1975, in “The Pharmacological Basis of Therapeutics”, Ch. 1).

Dosage amount and interval may be adjusted individually to provide plasma levels of the active compound which are sufficient to prevent or treat a disorder of the invention, depending on the particular situation. Dosages necessary to achieve these effects will depend on individual characteristics and route of administration.

Dosage intervals can also be determined using the value for the minimum effective concentration. Compounds should be administered using a regimen that maintains plasma levels above the minimum effective concentration for 10-90% of the time, preferably between 30-99%, and most preferably between 50-90%. In cases of local administration or selective uptake, the effective local concentration of the drug may not be related to plasma concentration.

The amount of composition administered will, of course, be dependent on the subject being treated, on the subject's weight, the severity of the affliction, the manner of administration, and the judgement of the prescribing physician.

A preferred dosage range for the amount of a modulator of the invention, which can be administered on a daily or regular basis to achieve desired results is 0.1-100 mg/kg body mass. Other preferred dosage range is 0.1-30 mg/kg body mass. Other preferred dosage range is 0.1-10 mg/kg body mass. Other preferred dosage range is 0.1-3.0 mg/kg body mass. Of course, these daily dosages can be delivered or administered in small amounts periodically during the course of a day. It is noted that these dosage ranges are only preferred ranges and are not meant to be limiting to the invention. Said desired results include, but are not limited to, lowering blood glucose concentration, preventing or treating certain metabolic disorders, such as insulin resistance, impaired glucose tolerance, and diabetes, and preventing or treating a complication of an elevated blood glucose concentration, such as atherosclerosis, heart disease, stroke, hypertension and peripheral vascular disease.

H. Methods of Treatment

The invention is drawn inter alia to methods including, but not limited to, methods of lowering blood glucose concentration, methods of preventing or treating certain metabolic disorders, such as insulin resistance and diabetes, and methods of preventing or treating a complication of an elevated blood glucose concentration, such as atherosclerosis, heart disease, stroke, hypertension and peripheral vascular disease, comprising providing an individual in need of such treatment with a modulator of the invention. In certain embodiments, the modulator is an agonist. In some embodiments, said modulator is orally bioavailable. In some embodiments, said orally bioavailable modulator is further able to cross the blood-brain barrier. In certain embodiments, the modulator is provided to the individual in a pharmaceutical or physiologically acceptable composition. In certain embodiments, the modulator is provided to the individual in a pharmaceutical composition. In certain embodiments, the modulator is provided to the individual in a physiologically acceptable composition. In certain embodiments, the modular is provided to the individual in a pharmaceutical or physiologically acceptable composition that is taken orally. In certain embodiments, the individual is a non-human mammal. In certain embodiments, the individual is a mammal. In certain embodiments, the individual or mammal is a human.

In certain embodiments, the metabolic disorder is selected from the group consisting of impaired glucose tolerance, insulin resistance, hyperinsulinemia, and diabetes. In some embodiments, diabetes is type 1 diabetes. In certain preferred embodiments, diabetes is type 2 diabetes. In certain embodiments, the metabolic disorder is diabetes. In certain embodiments, the metabolic disorder is type 1 diabetes. In certain embodiments, the metabolic disorder is type 2 diabetes. In certain embodiments, the metabolic disorder is impaired glucose tolerance. In certain embodiments, the metabolic disorder is insulin resistance. In certain embodiments, the metabolic disorder is hyperinsulinemia. In certain embodiments, the metabolic disorder is related to an elevated blood glucose concentration in the individual.

In certain embodiments, the complication of an elevated blood glucose concentration is selected from the group consisting of Syndrome X, atherosclerosis, atheromatous disease, heart disease, hypertension, stroke, neuropathy, retinopathy, nephropathy, and peripheral vascular disease. Heart disease includes, but is not limited to, cardiac insufficiency, coronary insufficiency, coronary artery disease, and high blood pressure. In certain embodiments, the complication is Syndrome X. In certain embodiments, the complication is atherosclerosis. In certain embodiments, the complication is atheromatous disease. In certain embodiments, the complication is heart disease. In certain embodiments, the complication is cardiac insufficiency. In certain embodiments, the complication is coronary insufficiency. In certain embodiments, the complication is coronary artery disease. In certain embodiments, the complication is high blood pressure. In certain embodiments, the complication is hypertension. In certain embodiments, the complication is stroke. In certain embodiments, the complication is neuropathy. In certain embodiments, the complication is retinopathy. In certain embodiments, the complication is neuropathy. In certain embodiments, the complication is peripheral vascular disease. In certain embodiments, the complication is polycystic ovary syndrome. In certain embodiments, the complication is hyperlipidemia.

L. Other Utility

Agents that modulate (i.e., increase, decrease, or block) RUP43 receptor functionality may be identified by contacting a candidate compound with a RUP43 receptor and determining the effect of the candidate compound on RUP43 receptor functionality. The selectivity of a compound that modulates the functionality of the RUP43 receptor can be evaluated by comparing its effects on the RUP43 receptor to its effects on other G protein-coupled receptors. By way of illustration and not limitation, a modulator of an endogenous RUP43 receptor can be shown to be selective in comparison with one or more other endogenous G protein-coupled receptors from the same species. By way of illustration and not limitation, an agonist of an endogenous RUP43 receptor can be shown to be a selective RUP43 agonist if the EC50 of the agonist on the endogenous RUP43 receptor is at least 100-fold lower than the EC50 of the agonist on one or more other endogenous G protein-coupled receptors from the same species. Following identification of compounds that modulate RUP43 receptor functionality, such candidate compounds may be further tested in other assays including, but not limited to, in vivo models, in order to confirm or quantitate their activity. Modulators of RUP43 receptor functionality are therapeutically useful in treatment of diseases and physiological conditions in which normal or aberrant RUP43 receptor functionality is involved.

Agents that are ligands of RUP43 receptor may be identified by contacting a candidate compound with a RUP43 receptor and determining whether the candidate compound binds to the RUP43 receptor. The selectivity of a compound that binds to the RUP43 receptor can be evaluated by comparing its binding to the RUP43 receptor to its binding on other receptors. By way of illustration and not limitation, a ligand of an endogenous RUP43 receptor can be shown to be selective in comparison with one or more other endogenous G protein-coupled receptors from the same species. Ligands that are modulators of RUP43 receptor functionality are therapeutically useful in treatment of diseases and physiological conditions in which normal or aberrant RUP43 receptor functionality is involved.

The present invention also relates to radioisotope-labeled versions of compounds of the invention identified as modulators or ligands of RUP43 receptor that would be useful not only in radio-imaging but also in assays, both in vitro and in vivo, for localizing and quantitating RUP43 receptor in tissue samples, including human, and for identifying RUP43 receptor ligands by inhibition binding of a radioisotope-labeled compound. It is a further object of this invention to develop novel RUP43 receptor assays which comprise such radioisotope-labeled compounds.

The present invention embraces radioisotope-labeled versions of compounds of the invention identified as modulators or ligands of RUP43 receptor.

The present invention also relates to radioisotope-labeled versions of test ligands that are useful for detecting a ligand bound to RUP43 receptor. In some embodiments, the present invention expressly contemplates a library of said radiolabeled test ligands useful for detecting a ligand bound to RUP43 receptor. In certain embodiments, said library comprises at least about 10, at least about 10², at least about 10³, at least about 10⁵, or at least about 10⁶ said radiolabeled test compounds. It is a further object of this invention to develop novel RUP43 receptor assays which comprise such radioisotope-labeled test ligands.

In some embodiments, a radioisotope-labeled version of a compound is identical to the compound, but for the fact that one or more atoms are replaced or substituted by an atom having an atomic mass or mass number different from the atomic mass or mass number typically found in nature (i.e., naturally occurring). Suitable radionuclides that may be incorporated in compounds of the present invention include but are not limited to ²H (deuterium), ³H (tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled compound will depend on the specific application of that radio-labeled compound. For example, for in vitro RUP43 receptor labeling and competition assays, compounds that incorporate ³H, ¹⁴C, ⁸²Br, ¹²⁵I, ¹³¹I, ³⁵S or will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful. In some embodiments, the radionuclide is selected from the group consisting of ³H, ¹¹C, ⁸⁸F, ¹⁴C, ¹²⁵I, ¹²⁴I, ¹³¹I, ³⁵S and ⁸²Br.

Synthetic methods for incorporating radio-isotopes into organic compounds are applicable to compounds of the invention and are well known in the art. These synthetic methods, for example, incorporating activity levels of tritium into target molecules, are as follows:

A. Catalytic Reduction with Tritium Gas—This procedure normally yields high specific activity products and requires halogenated or unsaturated precursors.

B. Reduction with Sodium Borohydride [³H]—This procedure is rather inexpensive and requires precursors containing reducible functional groups such as aldehydes, ketones, lactones, esters, and the like.

C. Reduction with Lithium Aluminum Hydride [³H ]—This procedure offers products at almost theoretical specific activities. It also requires precursors containing reducible functional groups such as aldehydes, ketones, lactones, esters, and the like.

D. Tritium Gas Exposure Labeling—This procedure involves exposing precursors containing exchangeable protons to tritium gas in the presence of a suitable catalyst.

E. N-Methylation using Methyl Iodide [³H]—This procedure is usually employed to prepare O-methyl or N-methyl (³H) products by treating appropriate precursors with high specific activity methyl iodide (³H). This method in general allows for higher specific activity, such as for example, about 70-90 Ci/mmol.

Synthetic methods for incorporating activity levels of ¹²⁵I into target molecules include:

A. Sandmeyer and like reactions—This procedure transforms an aryl or heteroaryl amine into a diazonium salt, such as a tetrafluoroborate salt, and subsequently to ¹²⁵I labeled compound using Na¹²⁵I. A represented procedure was reported by Zu, D.-G. and co-workers in J. Org. Chem. 2002, 67,943-948.

B. Ortho ¹²⁵Iodination of phenols—This procedure allows for the incorporation of ¹²⁵I at the ortho position of a phenol as reported by Collier, T. L. and co-workers in J. Labeled Compd Radiopharm. 1999, 42, S264-S266.

C. Aryl and heteroaryl bromide exchange with ¹²⁵I—This method is generally a two step process. The first step is the conversion of the aryl or heteroaryl bromide to the corresponding tri-alkyltin intermediate using for example, a Pd catalyzed reaction [i.e. Pd(Ph₃P)₄] or through an aryl or heteroaryl lithium, in the presence of a tri-alkyltinhalide or hexaalkylditin [e.g., (CH₃)₃SnSn(CH₃)₃]. A represented procedure was reported by Bas, M.-D. and co-workers in J. Labeled Compd Radiopharm. 2001, 44, S280-S282.

In some embodiments, a radioisotope-labeled version of a compound is identical to the compound, but for the addition of one or more substituents comprising a radionuclide. In some further embodiments, the compound is a polypeptide. In some further embodiments, the compound is an antibody or an antigen-binding fragment thereof. In some further embodiments, said antibody is monoclonal. Suitable said radionuclide includes but is not limited to ²H (deuterium), ³H (tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled compound will depend on the specific application of that radio-labeled compound. For example, for in vitro RUP43 receptor labeling and competition assays, compounds that incorporate ³H, ¹⁴C, ⁸²Br, ¹²⁵I, ¹³¹I, ³⁵S or will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful. In some embodiments, the radionuclide is selected from the group consisting of ³H, ¹¹C, ¹⁸F, ¹⁴C, ¹²⁵I, ¹²⁴I, ¹³¹I, ³⁵S and ⁸²Br.

Methods for adding one or more substituents comprising a radionuclide are within the purview of the skilled artisan and include, but are not limited to, addition of radioisotopic iodine by enzymatic method [Marchalonic J J, Biochemical Journal (1969) 113:299-305; Thorell J I and Johansson B G, Biochimica et Biophysica Acta (1969) 251:363-9; the disclosure of each of which is hereby incorporated by reference in its entirety] and or by Chloramine-T/Iodogen/Iodobead methods [Hunter W M and Greenwood F C, Nature (1962) 194:495-6; Greenwood F C et al., Biochemical Journal (1963) 89:114-23; the disclosure of each of which is hereby incorporated by reference in its entirety].

Other uses of the disclosed receptors and methods will become apparent to those in the art based upon, inter alia, a review of this patent document.

### EXAMPLES

The following examples are presented for purposes of elucidation, and not limitation, of the present invention. While specific nucleic acid and amino acid sequences are disclosed herein, those of ordinary skill in the art are credited with the ability to make minor modifications to these sequences while achieving the same or substantially similar results reported below. Such modified approaches are considered within the purview of this disclosure.

The following Examples are provided for illustrative purposes and not as a means of limitation. One of ordinary skill in the art would be able to design equivalent assays and methods based on the disclosure herein, all of which form part of the present invention.

Recombinant DNA techniques relating to the subject matter of the present invention and well known to those of ordinary skill in the art can be found, e.g, in Maniatis T et al., Molecular Cloning: A Laboratory Manual (1989) Cold Spring Harbor Laboratory; U.S. Pat. No. 6,399,373; and PCT Application No. PCT/JB02/01461 published as WO 02/066505 on 29 Aug. 2002; the disclosure of each of which is hereby incorporated by reference in its entirety.

### Example 1

Full-Length Cloning of Human GPCRs

Endogenous Human RUP43 (SEQ ID NOs:1 & 2)

Polynucleotide sequence encoding full-length endogenous human RUP43 (GPR131, e.g. GenBank® Accession No. NM_{—}170699) can be cloned as described here. SEQ ID NO:1 is an endogenous human RUP43 (GPR131) polynucleotide coding sequence that may be cloned as described here. SEQ ID NO:2 is the corresponding encoded endogenous human RUP43 (GPR131) polypeptide.

Full-length endogenous human RUP43 is cloned by PCR using Platinum PCR SuperMix (Invitrogen catalog # 11306-016) and the specific primers

5′-GACAAGCATGACGCCCAACAGCACTGGCGAG-3′ (5′-primer;
SEQ ID NO:3)
and
5′-CTTGAATTAGTTCAAGTCCAGGTCGACACTGC-3′ (3′-primer;
SEQ ID NO:4)

with human DNA as template. The human DNA may be genomic DNA or cDNA. The cycle condition used is 25 cycles of 95° C. for 40 sec, 60° C. for 50 sec, and 72° C. for 1 min. The 1.0 kb PCR product is cloned into the pCRlI-TOPO™ vector (Invitrogen).

HA/V5His Double Tagged Endogenous Human RUP43 (SEQ ID NOs:5 & 6)

Polynucleotide encoding full-length endogenous human RUP43 (GPR131) polypeptide (absent the N-terminal methionine) with N-terminal HA epitope tag and C-terminally disposed V5His epitope tag was cloned as described here. “HA” epitope tag comprises amino acid sequence MYPYDVPDYA. “V5” comprises amino acid sequence GKPIPNPLLGLDST; “His” comprises amino acid sequence HHHHHH. SEQ ID NO:5 is endogenous human RUP43 (GPR131) polynucleotide coding sequence (absent the codon encoding the N-terminal methionine) with 5′-terminal HA epitope tag and 3′-terminal V5His epitope tag. SEQ ID NO:6 is the corresponding encoded HA/V5His double-tagged RUP43 polypeptide.

PCR was performed using an EST clone (IMAGE #5221127, GenBank® Accession No. BC033625) as template and pfu polymerase (Stratagene), with the buffer system provided by the manufacturer supplemented with 10% DMSO, 0.25 μM of each primer, and 0.5 mM of each 4 nucleotides. The cycle condition was 25 cycles of 95° C. for 40 sec, 60° C. for 50 sec, and 72° C. for 1 min 40 sec. The 5′ PCR primer incorporated a HindIII site and had the sequence:

(SEQ ID NO:7)

5′-GACAAGCTTGACGCCCAACAGCACTGGCGAG-3′.

The 3′ PCR primer incorporated an EcoRI site and had the sequence:

(SEQ ID NO:8)

5′-CTTGAATTCGTTCAAGTCCAGGTCGACACTGC-3′.

The 1.0 kb PCR product was digested with HindIII and EcoRI and cloned into 5′HA/3′V5His double-tagged pCMV expression vector.

### Example 2

Preparation of Non-Endogenous, Constitutively Activated Human RUP43

Those skilled in the art are credited with the ability to select techniques for mutation of a nucleic acid sequence. Presented below are approaches that may be utilized to create non-endogenous versions of human GPCRs. The mutation disclosed here for endogenous human RUP43 (GPR131) is based upon an algorithmic approach whereby the 16^{th} amino acid (located in the IC3 region of the GPCR) N-terminal to a conserved proline (or an endogenous, conservative substitution therefor) residue (located in the TM6 region of the GPCR, near the TM6/IC3 interface) is mutated, preferably to a histidine, arginine or lysine amino acid residue, most preferably to a lysine amino acid residue.

By way of illustration and not limitation, a non-endogenous, constitutively activated version of endogenous human RUP43 (GPR131) may be made by mutating alanine at amino acid position 223 of SEQ ID NO:2, preferably to a lysine.

1. Transformer Site-Directed™ Mutagenesis

Preparation of non-endogenous human GPCRs may be accomplished on human GPCRs using, inter alia, Transformer Site-Directed™ Mutagenesis Kit (Clontech) according to the manufacturer instructions. Two mutagenesis primers are utilized, most preferably a lysine mutagenesis oligonucleotide that creates the lysine mutation, and a selection marker oligonucleotide. For convenience, the codon mutation to be incorporated into the human GPCR is also noted, in standard form

2. QuikChange™ Site-Directed™ Mutagenesis

Preparation of non-endogenous human GPCRs can also be accomplished by using QuikChange™ Site-Directed™ Mutagenesis Kit (Stratagene, according to manufacturer's instructions). Endogenous GPCR is preferably used as a template and two mutagenesis primers utilized, as well as, most preferably, a lysine mutagenesis oligonucleotide and a selection marker oligonucleotide (included in kit). For convenience, the codon mutation incorporated into the novel human GPCR and the respective oligonucleotides are noted, in standard form.

### Example 3

Receptor Expression

Although a variety of cells are available to the art for the expression of proteins, it is most preferred that mammalian cells or melanophores be utilized. The primary reason for this is predicated upon practicalities, i.e., utilization of e.g., yeast cells for the expression of a GPCR, while possible, introduces into the protocol a non-mammalian cell which may not (indeed, in the case of yeast, does not) include the receptor-coupling, genetic-mechanism and secretary pathways that have evolved for mammalian systems—thus, results obtained in non-mammalian cells, while of potential use, are not as preferred as that obtained from mammalian cells or melanophores. Of the mammalian cells, CHO, COS-7, MCB3901, 293 and 293T cells are particularly preferred, although the specific mammalian cell utilized can be predicated upon the particular needs of the artisan. In some embodiments, adipocytes or skeletal muscle cells obtained from a mammal may be used. See infra as relates to melanophores, including Example 10.

a Transient Transfection

On day one, 6×10⁶/10 cm dish of 293 cells are plated out On day two, two reaction tubes are prepared (the proportions to follow for each tube are per plate): tube A is prepared by mixing 4 μg DNA (e.g., pCMV vector; pCMV vector with receptor cDNA, etc.) in 0.5 ml serum free DMEM (Gibco BRL); tube B is prepared by mixing 24 μl lipofectamine (Gibco BRL) in 0.5 ml serum free DMEM Tubes A and B are admixed by inversions (several times), followed by incubation at room temperature for 30-45 min. The admixture is referred to as the “transfection mixture”. Plated 293 cells are washed with 1× PBS, followed by addition of 5 ml serum free DMEM. 1 ml of the transfection mixture is added to the cells, followed by incubation for 4 hrs at 37° C./5% CO₂. The transfection mixture is removed by aspiration, followed by the addition of 10 ml of DMEM/10% Fetal Bovine Serum. Cells are incubated at 37° C./5% CO₂. After 48 hr incubation, cells are harvested and utilized for analysis.

b. Stable Cell Lines

Approximately 12×10⁶ 293 cells are plated on a 15 cm tissue culture plate. Grown in DME High Glucose Medium containing ten percent fetal bovine serum and one percent sodium pyruvate, L-glutamine, and antibiotics. Twenty-four hours following plating of 293 cells (or to ˜80% confluency), the cells are transfected using 12 μg of DNA (e.g., pCMV vector with receptor cDNA). The 12 μg of DNA is combined with 60 μl of lipofectamine and 2 ml of DME High Glucose Medium without serum. The medium is aspirated from the plates and the cells are washed once with medium without serum. The DNA, lipofectamine, and medium mixture are added to the plate along with 10 ml of medium without serum. Following incubation at 37° C. for four to five hours, the medium is aspirated and 25 ml of medium containing serum is added. Twenty-four hours following transfection, the medium is aspirated again, and fresh medium with serum is added. Forty-eight hours following transfection, the medium is aspirated and medium with serum is added containing geneticin (G418 drug) at a final concentration of approximately 12×10⁶ 293 cells are plated on a 15 cm tissue culture plate. Grown in DME High Glucose Medium containing ten percent fetal bovine serum and one percent sodium pyruvate, L-glutamine, and antibiotics. Twenty-four hours following plating of 293 cells (or to ˜80% confluency), the cells are transfected using 12 μg of DNA (e.g., pCMV vector with receptor cDNA). The 12 μg of DNA is combined with 60 μl of lipofectamine and 2 ml of DMB High Glucose Medium without serum. The medium is aspirated from the plates and the cells are washed once with medium without serum. The DNA, lipofectamine, and medium mixture are added to the plate along with 10 mL of medium without serum. Following incubation at 37° C. for four to five hours, the medium is aspirated and 25 ml of medium containing serum is added. Twenty-four hours following transfection, the medium is aspirated again, and fresh medium with serum is added. Forty-eight hours following transfection, the medium is aspirated and medium with serum is added containing geneticin (G418 drug) at a final concentration of 500 μg/ml. The transfected cells now undergo selection for positively transfected cells containing the G418 resistance gene. The medium is replaced every four to five days as selection occurs. During selection, cells are grown to create stable pools, or split for stable clonal selection.

### Example 4

Assays for Determination of GPCR Activation

A variety of approaches are available for assessment of activation of human GPCRs. The following are illustrative; those of ordinary skill in the art are credited with the ability to determine those techniques that are preferentially beneficial for the needs of the artisan.

1. Membrane Binding Assays: [³⁵S]GTPγS Assay

When a G protein-coupled receptor is in its active state, either as a result of ligand binding or constitutive activation, the receptor couples to a G protein and stimulates the release of GDP and subsequent binding of GTP to the G protein. The alpha subunit of the G protein-receptor complex acts as a GTPase and slowly hydrolyzes the GTP to GDP, at which point the receptor normally is deactivated. Activated receptors continue to exchange GDP for GTP. The non-hydrolyzable GTP analog, [³⁵S]GTPγS, can be utilized to demonstrate enhanced binding of [³⁵S]GTPγS to membranes expressing activated receptors. The advantage of using [³⁵S]GTPγS binding to measure activation is that (a) it is generically applicable to all G protein-coupled receptors; (b) it is proximal at the membrane surface making it less likely to pick-up molecules which affect the intracellular cascade.

The assay utilizes the ability of G protein coupled receptors to stimulate [³⁵ S]GTPγS binding to membranes expressing the relevant receptors. The assay can, therefore, be used in the direct identification method to screen candidate compounds to endogenous GPCRs and non-endogenous, constitutively activated GPCRs. The assay is generic and has application to drug discovery at all G protein-coupled receptors.

The [³⁵S]GTPγS assay is incubated in 20 mM HEPES and between 1 and about 20 mM MgCl₂ (this amount can be adjusted for optimization of results, although 20 mM is preferred) pH 7.4, binding buffer with between about 0.3 and about 1.2 nM [³⁵S]GTPγS (this amount can be adjusted for optimization of results, although 1.2 is preferred ) and 12.5 to 75 μg membrane-protein (e.g., 293 cells expressing the Gs Fusion Protein; this amount can be adjusted for optimization) and 10 μM GDP (this amount can be changed for optimization) for 1 hour. Wheatgerm agglutinin beads (25 μl; Amersham) are then added and the mixture incubated for another 30 minutes at room temperature. The tubes are then centrifuged at 1500×g for 5 minutes at room temperature and then counted in a scintillation counter.

2. Adenylyl Cyclase

A Flash Plate™ Adenylyl Cyclase kit (New England Nuclear, Cat No. SMP004A) designed for cell-based assays can be modified for use with crude plasma membranes. The Flash Plate wells can contain a scintillant coating which also contains a specific antibody recognizing cAMP. The cAMP generated in the wells can be quantitated by a direct competition for binding of radioactive cAMP tracer to the cAMP antibody. The following serves as a brief protocol for the measurement of changes in cAMP levels in whole cells that express the receptors.

Transfected cells are harvested approximately twenty four hours after transient transfection. Media is carefully aspirated off and discarded. 10 ml of PBS is gently added to each dish of cells followed by careful aspiration. 1 ml of Sigma cell dissociation buffer and 3 ml of PBS are added to each plate. Cells are pipetted off the plate and the cell suspension is collected into a 50 ml conical centrifuge tube. Cells are then centrifuged at room temperature at 1,100 rpm for 5 min. The cell pellet is carefully re-suspended into an appropriate volume of PBS (about 3 ml/plate). The cells are then counted using a hemocytometer and additional PBS is added to give the appropriate number of cells (with a final volume of about 50 μl/well).

cAMP standards and Detection Buffer (comprising 1 μCi of tracer [¹²⁵I] cAMP (50 μl) to 11 ml Detection Buffer) is prepared and maintained in accordance with the manufacturer's instructions. Assay Buffer is prepared fresh for screening and contains 50 μl of Stimulation Buffer, 3 μl of test compound (12 μM final assay concentration) and 50 μl cells. Assay Buffer is stored on ice until utilized. The assay, preferably carried out e.g. in a 96-well plate, is initiated by addition of 50 μl of cAMP standards to appropriate wells followed by addition of 50 μl of PBSA to wells H-11 and H12. 50 μl of Stimulation Buffer is added to all wells. DMSO (or selected candidate compounds) is added to appropriate wells using a pin tool capable of dispensing 3 μl of compound solution, with a final assay concentration of 12 μM test compound and 100 μl total assay volume. The cells are then added to the wells and incubated for 60 min at room temperature. 100 μl of Detection Mix containing tracer cAMP is then added to the wells. Plates are then incubated additional 2 hours followed by counting in a Wallac MicroBeta scintillation counter. Values of cAMP/well are then extrapolated from a standard cAMP curve which is contained within each assay plate.

3. Cell-Based cAMP for Gi Coupled Target GPCRs

TSHR is a Gs coupled GPCR that causes the accumulation of cAMP upon activation. TSHR will be constitutively activated by mutating amino acid residue 623 (i.e., changing an alanine residue to an isoleucine residue). A Gi coupled receptor is expected to inhibit adenylyl cyclase, and, therefore, decrease the level of cAMP production, which can make assessment of cAMP levels challenging. An effective technique for measuring the decrease in production of cAMP as an indication of activation of a Gi coupled receptor can be accomplished by co-transfecting, most preferably, non-endogenous, constitutively activated TSHR (TSHR-A623I) (or an endogenous, constitutively active Gs coupled receptor) as a “signal enhancer” with a Gi linked target GPCR to establish a baseline level of cAMP. Upon creating a non-endogenous version of the Gi coupled receptor, this non-endogenous version of the target GPCR is then co-transfected with the signal enhancer, and it is this material that can be used for screening. We will utilize such approach to effectively generate a signal when a cAMP assay is used. In some embodiments, this approach is preferably used in the direct identification of candidate compounds against Gi coupled receptors. It is noted that for a Gi coupled GPCR, when this approach is used, an inverse agonist of the target GPCR will increase the cAMP signal and an agonist will decrease the cAMP signal.

On day one, 2×10⁴ 293 cells/well will be plated out. On day two, two reaction tubes will be prepared (the proportions to follow for each tube are per plate): tube A will be prepared by mixing 2 μg DNA of each receptor transfected into the mammalian cells, for a total of 4 μg DNA (e.g., pCMV vector, pCMV vector with mutated THSR (TSHR-A623I); TSHR-A6231 and GPCR, etc.) in 1.2 ml serum free DMEM (Irvine Scientific, Irvine, Calif.); tube B will be prepared by mixing 120 μl lipofectamine (Gibco BRL) in 1.2 ml serum free DMEM. Tubes A and B will then be admixed by inversions (several times), followed by incubation at room temperature for 30-45 min. The admixture is referred to as the “transfection mixture”. Plated 293 cells will be washed with 1× PBS, followed by addition of 10 ml serum free DMEM. 2.4 ml of the transfection mixture will then be added to the cells, followed by incubation for 4 hrs at 37° C./5% CO₂. The transfection mixture will then be removed by aspiration, followed by the addition of 25 ml of DMEM/10% Fetal Bovine Serum. Cells will then be incubated at 37° C./5% CO₂. After 24 hr incubation, cells will then be harvested and utilized for analysis.

A Flash Plate™ Adenylyl Cyclase kit (New England Nuclear, Cat No. SMP004A) is designed for cell-based assays, but can be modified for use with crude plasma membranes depending on the need of the skilled artisan. The Flash Plate wells will contain a scintillant coating which also contains a specific antibody recognizing cAMP. The cAMP generated in the wells can be quantitated by a direct competition for binding of radioactive cAMP tracer to the cAMP antibody. The following serves as a brief protocol for the measurement of changes in cAMP levels in whole cells that express the receptors.

Transfected cells will be harvested approximately twenty four hours after transient transfection. Media will be carefully aspirated off and discarded. 10 ml of PBS will be gently added to each dish of cells followed by careful aspiration 1 ml of Sigma cell dissociation buffer and 3 ml of PBS will be added to each plate. Cells will be pipetted off the plate and the cell suspension will be collected into a 50 ml conical centrifuge tube. Cells will then be centrifuged at room temperature at 1,100 rpm for 5 min. The cell pellet will be carefully re-suspended into an appropriate volume of PBS (about 3 ml/plate). The cells will then be counted using a hemocytometer and additional PBS is added to give the appropriate number of cells (with a final volume of about 50 μl/well).

cAMP standards and Detection Buffer (comprising 1 μCi of tracer [¹²⁵I] cAMP (50 μl) to 11 ml Detection Buffer) will be prepared and maintained in accordance with the manufacturer's instructions. Assay Buffer should be prepared fresh for screening and contained 50 μl of Stimulation Buffer, 3 μl of test compound (12 μM final assay concentration) and 50 μl cells, Assay Buffer can be stored on ice until utilized The assay can be initiated by addition of 50 μl of cAMP standards to appropriate wells followed by addition of 50 μl of PBSA to wells H-11 and H12. Fifty μl of Stimulation Buffer will be added to all wells. Selected compounds (e.g., TSH) will be added to appropriate wells using a pin tool capable of dispensing 3 μl of compound solution, with a final assay concentration of 12 μM test compound and 100 μl total assay volume. The cells will then be added to the wells and incubated for 60 min at room temperature. 100 μl of Detection Mix containing tracer cAMP will then be added to the wells. Plates were then incubated additional 2 hours followed by counting in a Wallac MicroBeta scintillation counter. Values of cAMP/well will then be extrapolated from a standard cAMP curve which is contained within each assay plate.

4. Reporter-Based Assays

CRE-LUC Reporter Assay (Gs-Associated Receptors)

293 and 293T cells are plated-out on 96 well plates at a density of 2×10⁴ cells per well and were transfected using Lipofectamine Reagent (BRL) the following day according to manufacturer instructions. A DNA/lipid mixture is prepared for each 6-well transfection as follows: 260 ng of plasmid DNA in 100 μl of DMEM is gently mixed with 24 of lipid in 100 μl of DMEM (the 260 ng of plasmid DNA consists of 200 ng of a 8×CRE-Luc reporter plasmid, 50 ng of pCMV comprising endogenous receptor or non-endogenous receptor or pCMV alone, and long of a GPRS expression plasmid (GPRS in pcDNA3 (Invitrogen)). The 8×CRE-Luc reporter plasmid was prepared as follows: vector SRIF-β-gal was obtained by cloning the rat somatostatin promoter (−71/+51) at BgIV-HindIII site in the pβgal-Basic Vector (Clontech). Eight (8) copies of cAMP response element were obtained by PCR from an adenovirus template AdpCF126CCRE8 [see, Suzuki et al., Hum Gene Ther (1996) 7:1883-1893; the disclosure of which is hereby incorporated by reference in its entirety) and cloned into the SRIF-β-gal vector at the Kpn-BgIV site, resulting in the 8×CRE-β-gal reporter vector. The 8×CRE-Luc reporter plasmid was generated by replacing the beta-galactosidase gene in the 8×CRE-β-gal reporter vector with the luciferase gene obtained from the pGL3-basic vector (Promega) at the HindIII-BamHI site. Following 30 min. incubation at room temperature, the DNA/lipid mixture is diluted with 400 μl of DMEM and 100 μl of the diluted mixture is added to each well. 100 μl of DMEM with 10% FCS are added to each well after a 4 hr incubation in a cell culture incubator. The following day the transfected cells are changed with 200 μl/well of DMEM with 10% FCS. Eight (8) hours later, the wells are changed to 100 μl/well of DMEM without phenol red, after one wash with PBS. Luciferase activity is measured the next day using the LucLite™ reporter gene assay kit (Packard) following manufacturer instructions and read on a 1450 MicroBeta™ scintillation and luminescence counter (Wallac).

b. AP1 Reporter Assay (Gq-Associated Receptors)

A method to detect Gq stimulation depends on the known property of Gq-dependent phospholipase C to cause the activation of genes containing AP1 elements in their promoter. A Pathdetect™ AP-1 cis-Reporting System (Stratagene, Catalogue # 219073) can be utilized following the protocol set forth above with respect to the CREB reporter assay, except that the components of the calcium phosphate precipitate were 410 ng pAP1-Luc, 80 ng pCMV-receptor expression plasmid, and 20 ng CMV-SEAP.

c. SRF-LUC Reporter Assay (Gq-Associated Receptors)

One method to detect Gq stimulation depends on the known property of Gq-dependent phospholipase C to cause the activation of genes containing serum response factors in their promoter. A Pathdetect™ SRF-Luc-Reporting System (Stratagene) can be utilized to assay for Gq coupled activity in, e.g., COS7 cells. Cells are transfected with the plasmid components of the system and the indicated expression plasmid encoding endogenous or non-endogenous GPCR using a Mammalian Transfection™ Kit (Stratagene, Catalogue #200285) according to the manufacturer's instructions. Briefly, 410 ng SRF-Luc, 80 ng pCMV-receptor expression plasmid and 20 ng CMV-SEAP (secreted alkaline phosphatase expression plasmid; alkaline phosphatase activity is measured in the media of transfected cells to control for variations in transfection efficiency between samples) are combined in a calcium phosphate precipitate as per the manufacturer's instructions. Half of the precipitate is equally distributed over 3 wells in a 96-well plate, kept on the cells in a serum free media for 24 hours. The last 5 hours the cells are incubated with, e.g. 1 μM, test compound. Cells are then lysed and assayed for luciferase activity using a Luclite™ Kit (Packard, Cat # 6016911) and ∫Trilux 1450 Microbeta” liquid scintillation and luminescence counter (Wallac) as per the manufacturer's instructions. The data can be analyzed using GraphPad Prism™ 2.0a (GraphPad Software Inc.).

Intracellular IP3 Accumulation Assay (Gq-Associated Receptors)

On day 1, cells comprising the receptors (endogenous or nonendogenous) can be plated onto 24 well plates, usually 1×10⁵ cells/well (although his number can be optimized. On day 2 cells can be transfected by first mixing 0.25 μg DNA in 50 μl serum free DMEM/well and 2 μl lipofectamine in 50 μl serum free DMEM/well. The solutions are gently mixed and incubated for 15-30 min at room temperature. Cells are washed with 0.5 ml PBS and 400 μl of serum free media is mixed with the transfection media and added to the cells. The cells are then incubated for 3-4 hrs at 37° C./5% CO₂ and then the transfection media is removed and replaced with 1 ml/well of regular growth media. On day 3 the cells are labeled with ³H-myo-inositol. Briefly, the media is removed and the cells are washed with 0.5 ml PBS. Then 0.5 ml inositol-free/serum free media (GIBCO BRL) is added/well with 0.25 μCi of ³H-myo-inositol/well and the cells are incubated for 16-18 hrs o/n at 37° C./5% CO₂. On Day 4 the cells are washed with 0.5 ml PBS and 0.45 ml of assay medium is added containing inositol-free/serum free media 10 μM pargyline 10 mM lithium chloride or 0.4 ml of assay medium and 50 μl of 10×ketanserin (ket) to final concentration of 10 μM. The cells are then incubated for 30 min at 37° C. The cells are then washed with 0.5 ml PBS and 200 μl of fresh/ice cold stop solution (1M KOH; 18 mM Na-borate; 3.8 mM EDTA) is added/well. The solution is kept on ice for 5-10 min or until cells were lysed and then neutralized by 200 μl of fresh/ice cold neutralization sol. (7.5 % HCl). The lysate is then transferred into 1.5 ml eppendorf tubes and 1 ml of chloroform/methanol (1:2) is added/tube. The solution is vortexed for 15 sec and the upper phase is applied to a Biorad AG1-X8™ anion exchange resin (100-200 mesh). Firstly, the resin is washed with water at 1:1.25 W/V and 0.9 ml of upper phase is loaded onto the column. The column is washed with 10 mls of 5 mM myo-inositol and 10 ml of 5 mM Na-borate/60 mM Na-formate. The inositol tris phosphates are eluted into scintillation vials containing 10 ml of scintillation cocktail with 2 ml of 0.1 M formic acid/1 M ammonium formate. The columns are regenerated by washing with 10 ml of 0.1 M formic acid/3M ammonium formate and rinsed twice with dd H₂O and stored at 4° C. in water.

### Example 5

Fusion Protein Preparation
a GPCR:Gs Fusion Constuct

The design of the GPCR-G protein fusion construct can be accomplished as follows: both the 5′ and 3′ ends of the rat G protein Gsα (long form; Itoh, H. et al., 83 PNAS 3776 (1986)) are engineered to include a HindIII (5′-AAGCTI-3′) sequence thereon. Following confirmation of the correct sequence (including the flanking HindIII sequences), the entire sequence is shuttled into pcDNA3.1(−) (Invitrogen, cat. no. V795-20) by subcloning using the HindIII restriction site of that vector. The correct orientation for the Gsα sequence is defined after subcloning into pcDNA3.1(−). The modified pcDNA3.1(−) containing the rat Gsα gene at HindIII sequence is then verified; this vector is now available as a “universal” Gsα protein vector. The pcDNA3.1(−) vector contains a variety of well-known restriction sites upstream of the HindIII site, thus beneficially providing the ability to insert, upstream of the Gs protein, the coding sequence of an endogenous, constitutively active GPCR. This same approach can be utilized to create other “universal” G protein vectors, and, of course, other commercially available or proprietary vectors known to the artisan can be utilized—the important criteria is that the sequence for the GPCR be upstream and in-frame with that of the G protein.

Gq(6 Amino Acid Deletion)/Gi Fusion Construct

The design of a Gq(de1)/Gi fusion construct can be accomplished as follows: the N-terminal six (6) amino acids (amino acids 2 through 7, having the sequence of TLESIM) of Gαq-subunit will be deleted and the C-terminal five (5) amino acids having the sequence EYNLV will be replaced with the corresponding amino acids of the Gαi Protein, having the sequence DCGLF. This fusion construct will be obtained by PCR using the following primers:

(SEQ ID NO:9)

5′-gatcAAGCTTCCATGGCGTGCTGCCTGAGCGAGGAG-3′
and

(SEQ ID NO:10)

5′-gatcGGATCCTTAGAACAGGCCGCAGTCCTTCAGGTTCAGCTGCAGG
ATGGTG-3′

and Plasmid 63313 which contains the mouse Gαq-wild type version with a hemagglutinin tag as template. Nucleotides in lower caps are included as spacers.

TaqPlus Precision DNA polymerase (Stratagene) will be utilized for the amplification by the following cycles, with steps 2 through 4 repeated 35 times: 95° C. for 2 min; 95° C. for 20 sec; 56° C. for 20 sec; 72° C. for 2 min; and 72° C. for 7 min. The PCR product will be cloned into a pCRII-TOPO vector (Invitrogen) and sequenced using the ABI Big Dye Terminator kit (P.E. Biosystems). Inserts from a TOPO clone containing the sequence of the fusion construct will be shuttled into the expression vector pcDNA3.1(+) at the HindIII/BamHI site by a 2 step cloning process. Also see, PCT Application No. PCT/US02/05625 published as WO02068600 on 6 Sep. 2002, the disclosure of which is hereby incorporated by reference in its entirety.

### Example 6

[³⁵S]GTPγS ASSAY
A. Membrane Preparation

In some embodiments membranes comprising the Target GPCR of interest and for use in the identification of candidate compounds as, e.g., inverse agonists, agonists, or antagonists, are preferably prepared as follows:

a. Materials

“Membrane Scrape Buffer” is comprised of 20 mM HEPES and 10 mM EDTA, pH 7.4; “Membrane Wash Buffer” is comprised of 20 mM HEPES and 0.1 mM EDTA, pH 7.4; “Binding Buffer” is comprised of 20 mM HEPES,100 mM NaCl, and 10 mM MgCl₂, pH 7.4.

b. Procedure

All materials will be kept on ice throughout the procedure. Firstly, the media will be aspirated from a confluent monolayer of cells, followed by rinse with 10 ml cold PBS, followed by aspiration. Thereafter, 5 ml of Membrane Scrape Buffer will be added to scrape cells; this will be followed by transfer of cellular extract into 50 ml centrifuge tubes (centrifuged at 20,000 rpm for 17 minutes at 4° C.). Thereafter, the supernatant will be aspirated and the pellet will be resuspended in 30 ml Membrane Wash Buffer followed by centrifuge at 20,000 rpm for 17 minutes at 4° C. The supernatant will then be aspirated and the pellet resuspended in Binding Buffer. This will then be homogenized using a Brinkman Polytron™ homogenizer (15-20 second bursts until the all material is in suspension). This is referred to herein as “Membrane Protein”.

Bradford Protein Assay

Following the homogenization, protein concentration of the membranes will be determined using the Bradford Protein Assay (protein can be diluted to about 1.5 mg/ml, aliquoted and frozen (−80° C.) for later use; when frozen, protocol for use will be as follows: on the day of the assay, frozen Membrane Protein is thawed at room temperature, followed by vortex and then homogenized with a Polytron at about 12×1,000 rpm for about 5-10 seconds; it is noted that for multiple preparations, the homogenizer should be thoroughly cleaned between homogenization of different preparations);
a. Materials

Binding Buffer (as per above); Bradford Dye Reagent; Bradford Protein Standard will be utilized, following manufacturer instructions (Biorad, cat no. 500-0006).
b. Procedure

Duplicate tubes will be prepared, one including the membrane, and one as a control “blank”. Each contained 800 μl Binding Buffer. Thereafter, 10 μl of Bradford Protein Standard (1 mg/ml) will be added to each tube, and 10 μl of membrane Protein will then be added to just one tube (not the blank). Thereafter, 200 μl of Bradford Dye Reagent will be added to each tube, followed by vortex of each. After five (5) minutes, the tubes will be re-vortexed and the material therein will be transferred to cuvettes. The cuvettes will then be read using a CECIL 3041 spectrophotometer, at wavelength 595.

Identification Assay

a. Materials

GDP Buffer consisted of 37.5 ml Binding Buffer and 2 mg GDP (Sigma, cat no. G-7127), followed by a series of dilutions in Binding Buffer to obtain 0.2 μM GDP (final concentration of GDP in each well was 0.1 μM GDP); each well comprising a candidate compound, has a final volume of 200 μl consisting of 100μl GDP Buffer (final concentration, 0.1 μM GDP), 50 μl Membrane Protein in Binding Buffer, and 50 μl [³⁵S]GTPγS (0.6 nM) in Binding Buffer (2.5 μl [³⁵S]GTPγS per 10 ml Binding Buffer).
b. Procedure

Candidate compounds will be preferably screened using a 96-well plate format (these can be frozen at −80° C.). Membrane Protein (or membranes with expression vector excluding the Target GPCR, as control), will be homogenized briefly until in suspension. Protein concentration will then be determined using the Bradford Protein Assay set forth above. Membrane Protein (and control) will then be diluted to 0.25 mg/ml in Binding Buffer (final assay concentration, 12.5 μg/well). Thereafter, 100 μl GDP Buffer was added to each well of a Wallac Scintistrip™ (Wallac). A 5 μl pin-tool will then be used to transfer 5 μl of a candidate compound into such well (i.e., 5 μl in total assay volume of 200 μl is a 1:40 ratio such that the final screening concentration of the candidate compound is 10 μM). Again, to avoid contamination, after each transfer step the pin tool should be rinsed in three reservoirs comprising water (1×), ethanol (1×) and water (2×)—excess liquid should be shaken from the tool after each rinse and dried with paper and kinwipes. Thereafter, 50 μl of Membrane Protein will be added to each well (a control well comprising membranes without the Target GPCR was also utilized), and pre-incubated for 5-10 minutes at room temperature. Thereafter, 50 μl of [³⁵S]GTPγS (0.6 nM) in Binding Buffer will be added to each well, followed by incubation on a shaker for 60 minutes at room temperature (again, in this example, plates were covered with foil). The assay will then be stopped by spinning of the plates at 4000 RPM for 15 minutes at 22° C. The plates will then be aspirated with an 8 channel manifold and sealed with plate covers. The plates will then be read on a Wallac 1450 using setting “Prot. #37” (as per manufacturer's instructions).

### Example 7

CYCLIC AMP ASSAY

Another assay approach for identifying candidate compounds as, e.g., inverse agonists, agonists, or antagonists, is accomplished by utilizing a cyclase-based assay. In addition to direct identification, this assay approach can be utilized as an independent approach to provide confirmation of the results from the [³⁵S]GTPγS approach as set forth in Example 6, supra.

A modified Flash Plate™ Adenylyl Cyclase kit (New England Nuclear, Cat No. SMP004A) is preferably utilized for direct identification of candidate compounds as inverse agonists and agonists to endogenous or non-endogenous, constitutively actived GPCRs in accordance with the following protocol.

Transfected cells are harvested approximately three days after transfection. Membranes are prepared by homogenization of suspended cells in buffer containing 20 mM HEPES, pH 7.4 and 10 mM MgCl₂. Homogenization is performed on ice using a Brinkman Polytron™ for approximately 10 seconds. The resulting homogenate is centrifuged at 49,000×g for 15 minutes at 4° C. The resulting pellet is then resuspended in buffer containing 20 mM HEPES, pH 7.4 and 0.1 mM EDTA, homogenized for 10 seconds, followed by centrifugation at 49,000×g for 15 minutes at 4° C. The resulting pellet is then stored at −80° C. until utilized. On the day of direct identification screening, the membrane pellet is slowly thawed at room temperature, resuspended in buffer containing 20 mM HEPES, pH 7.4 and 10 mM MgCl₂, to yield a final protein concentration of 0.60 mg/ml (the resuspended membranes are placed on ice until use).

cAMP standards and Detection Buffer (comprising 2 μCi of tracer {[¹²⁵I]cAMP (100 μl) to 11 ml Detection Buffer] are prepared and maintained in accordance with the manufacturer's instructions. Assay Buffer was prepared fresh for screening and contained 20 mM HEPES, pH 7.4, 10 mM MgCl₂ 20 mM phospocreatine (Sigma), 0.1 units/ml creatine phosphokinase (Sigma), 50 μM GTP (Sigma), and 0.2 mM ATP (Sigma); Assay Buffer was then stored on ice until utilized.

Candidate compounds are added, preferably, to e.g. 96-well plate wells (3 μl/well, 12 μM final assay concentration), together with 40 μl Membrane Protein (30 μg/well) and 50 μl of Assay Buffer. This admixture was then incubated for 30 minutes at room temperature, with gentle shaking.

Following the incubation, 100 μl of Detection Buffer is added to each well, followed by incubation for 2-24 hours. Plates are then counted in a Wallac MicroBeta™ plate reader using “Prot. #31” (as per manufacturer's instructions).

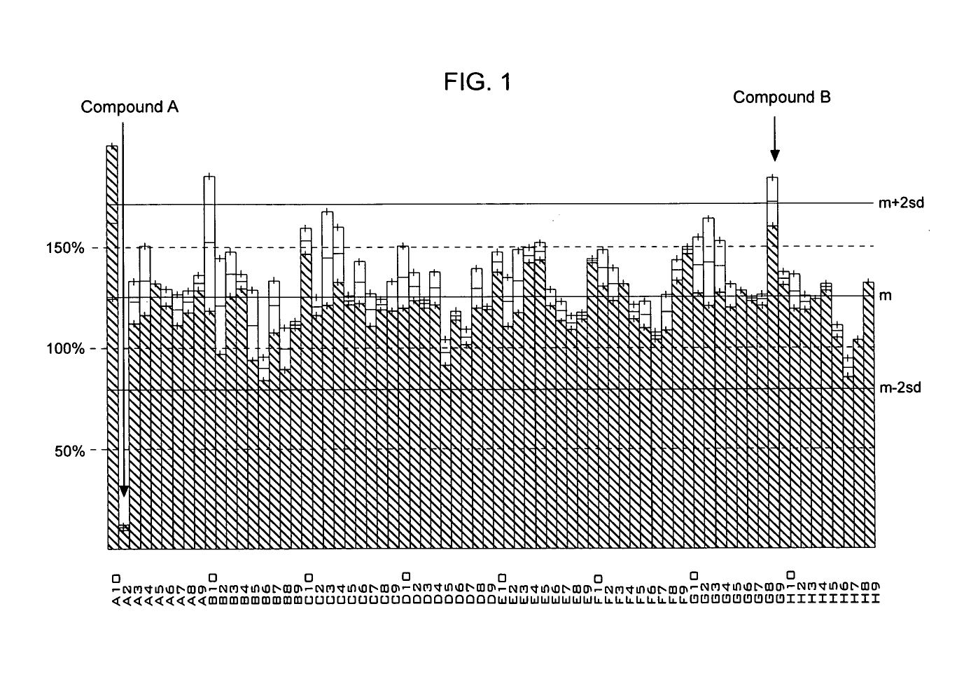

By way of example and not limitation, an illustrative screening assay plate (96 well format) result obtained is presented in FIG. 1. Each bar represents the result for a compound that differs in each well, the “Target GPCR” being a Gsα Fusion Protein construct of an endogenous, constitutively active Gs-coupled GPCR unrelated to GPR131. The results presented in FIG. 1 also provide standard deviations based upon the mean results of each plate (“m”) and the mean plus two arbitrary preference for selection of inverse agonists as “leads” from the primary screen involves selection of candidate compounds that that reduce the per cent response by at least the mean plate response, minus two standard deviations. Conversely, an arbitrary preference for selection of agonists as “leads” from the primary screen involves selection of candidate compounds that increase the per cent response by at least the mean plate response, plus the two standard deviations. Based upon these selection processes, the candidate compounds in the following wells were directly identified as putative inverse agonist (Compound A) and agonist (Compound B) to said endogenous GPCR in wells A2 and G9, respectively. See, FIG. 1. It is noted for clarity: these compounds have been directly identified without any knowledge of the endogenous ligand for this GPCR. By focusing on assay techniques that are based upon receptor function, and not compound binding affinity, it is possible to ascertain compounds that are able to reduce the functional activity of this receptor (Compound A) as well as increase the functional activity of the receptor (Compound B).

### Example 8

Fluorometric Imaging Plate Reader (FLIPR) Assay for the Measurement of Intracellular Calcium Concentration

Target Receptor (experimental) and pCMV (negative control) stably transfected cells from respective clonal lines are seeded into poly-D-lysine pretreated 96-well plates (Becton-Dickinson, #356640) at 5.5×10⁴ cells/well with complete culture medium (DMEM with 10% FBS, 2 mM L-glutamine, 1 mM sodium pyruvate) for assay the next day. To prepare Fluo4-AM (Molecular Probe, #F14202) incubation buffer stock, 1 mg Fluo4-AM is dissolved in 467 μl DMSO and 467 μl Pluoronic acid (Molecular Probe, #P3000) to give a 1 mM stock solution that can be stored at −20° C. for a month Fluo4-AM is a fluorescent calcium indicator dye.

Candidate compounds are prepared in wash buffer (1× HBSS/2.5 mM Probenicid/20 mM HEPES at pH 7.4).

At the time of assay, culture medium is removed from the wells and the cells are loaded with 100 μl of 4 μM Fluo4-AM/2.5 mM Probenicid (Sigma, #P8761)/20 mM HEPES/complete medium at pH 7.4. Incubation at 37° C./5% CO2 is allowed to proceed for 60 min.

After the 1 hr incubation, the Fluo4-AM incubation buffer is removed and the cells are washed 2× with 100 μl wash buffer. In each well is left 100 μl wash buffer. The plate is returned to the incubator at 37° C./5% CO2 for 60 min.

FLIPR (Fluorometric Imaging Plate Reader, Molecular Device) is programmed to add 50 μl candidate compound on the 30th second and to record transient changes in intracellular calcium concentration ([Ca2+]) evoked by the candidate compound for another 150 seconds. Total fluorescence change counts are used to determine agonist activity using the FLIPR software. The instrument software normalizes the fluorescent reading to give equivalent initial readings at zero.

In some embodiments, the cells comprising Target Receptor further comprise Gα15, Gα16, or the chimeric Gq/Gi alpha unit.

Although the foregoing provides a FLIPR assay for agonist activity using stably transfected cells, a person of ordinary skill in the art would readily be able to modify the assay in order to characterize antagonist activity. Said person of ordinary skill in the art would also readily appreciate that, alternatively, transiently transfected cells could be used.

### Example 9

MAP Kinase Assay

MAP kinase (mitogen activated kinase) may be monitored to evaluate receptor activation. MAP kinase can be detected by several approaches. One approach is based on an evaluation of the phosphorylation state, either unphosphorylated (inactive) or phosphorylated (active). The phosphorylated protein has a slower mobility in SDS-PAGE and can therefore be compared with the unstimulated protein using Western blotting. Alternatively, antibodies specific for the phosphorylated protein are available (New England Biolabs) which can be used to detect an increase in the phosphorylated kinase. In either method, cells are stimulated with the test compound and then extracted with Laemmli buffer. The soluble fraction is applied to an SDS-PAGE gel and proteins are transferred electrophoretically to nitrocellulose or Immobilin. Immunoreactive bands are detected by standard Western blotting technique. Visible or chemiluminescent signals are recorded on film and may be quantified by densitometry.

Another approach is based on evalulation of the MAP kinase activity via a phosphorylation assay. Cells are stimulated with the test compound and a soluble extract is prepared. The extract is incubated at 30° C. for 10 min with gamma-³²P-ATP, an ATP regenerating system, and a specific substrate for MAP kinase such as phosphorylated heat and acid stable protein regulated by insulin, or PHAS-I. The reaction is terminated by the addition of H₃PO₄ and samples are transferred to ice. An aliquot is spotted onto Whatman P81 chromatography paper, which retains the phosphorylated protein. The chromatography paper is washed and counted for ³²P is a liquid scintillation counter. Alternatively, the cell extract is incubated with gamma-³²P-ATP, an ATP regeneratng system and biotinylated myelin basic proein bound by streptavidin to a filter support. The myelin basic protein is a substrate for activated MAP kinase. The phosphorylation reaction is carried out for 10 min at 30° C. The extract can then be aspirated through the filter, which retains, the phosphorylated myelin basic protein. The filter is washed and counted for 32P by liquid scintillation counting.

### Example 10

Melanophore Technology

Melanophores are skin cells found in lower vertebrates. They contain pigmented organelles termed melanosomes. Melanophores are able to redistribute these melanosomes along a microtubule network upon G-protein coupled receptor (GPCR) activation. The result of this pigment movement is an apparent lightening or darkening of the cells. In melanophores, the decreased levels of intracellular cAMP that result from activation of a Gi-coupled receptor cause melanosomes to migrate to the center of the cell, resulting in a dramatic lightening in color. If cAMP levels are then raised, following activation of a Gs-coupled receptor, the melanosomes are re-dispersed and the cells appear dark again. The increased levels of diacylglycerol that result from activation of Gq-coupled receptors can also induce this re-dispersion. In addition, the technology is also suited to the study of certain receptor tyrosine kinases. The response of the melanophores takes place within minutes of receptor activation and results in a simple, robust color change. The response can be easily detected using a conventional absorbance microplate reader or a modest video imaging system. Unlike other skin cells, the melanophores derive from the neural crest and appear to express a full complement of signaling proteins. In particular, the cells express an extremely wide range of G-proteins and so are able to functionally express almost all GPCRs.

Melanophores can be utilized to identify compounds, including natural ligands, against GPCRs. This method can be conducted by introducing test cells of a pigment cell line capable of dispersing or aggregating their pigment in response to a specific stimulus and expressing an exogenous clone coding for the GCPR A stimulant, e.g., melatonia, sets an initial state of pigment disposition wherein the pigment is aggregated within the test cells if activation of the GPCR induces pigment dispersion. However, stimulating the cell with a stimulant to set an initial state of pigment disposition wherein the pigment is dispersed if activation of the GPCR induces pigment aggregation. The test cells are then contacted with chemical compounds, and it is determined whether the pigment disposition in the cells changed from the initial state of pigment disposition. Dispersion of pigments cells due to the candidate compound, including but not limited to a ligand, coupling to the GPCR will appear dark on a petri dish, while aggregation of pigments cells will appear light

Materials and methods will be followed according to the disclosure of U.S. Pat. No. 5,462,856 and U.S. Pat. No. 6,051,386. These patent disclosures are hereby incorporated by reference in their entirety.

The cells are plated in e.g. 96-well plates (one receptor per plate). 48 hours post-transfection, half of the cells on each plate are treated with 10 nM melatonin. Melatonin activates an endogenous Gi-coupled receptor in the melanophores and causes them to aggregate their pigment. The remaining half of the cells are transferred to serum-free medium 0.7×L-15 (Gibco). After one hour, the cells in serum-free media remain in a pigment-dispersed state while the melatonin-treated cells are in a pigment-aggregated state. At this point, the cells are treated with a dose response of a test/candidate compound. If the plated GPCRs bind to the test/candidate compound, the melanophores would be expected to undergo a color change in response to the compound. If the receptor were either a Gs or Gq coupled receptor, then the melatonin-aggregated melanophores would undergo pigment dispersion. In contrast, if the receptor was a Gi-coupled receptor, then the pigment-dispersed cells would be expected to undergo a dose-dependent pigment aggregation.

### Example 11

Tissue Distribution of Human and Mouse RUP43

The expression of RUP43 by human and mouse adipocytes and skeletal muscle cells was interrogated by RT-PCR. The expression of RUP43 by human leukocyte subsets was interrogated by TaqMan RT-PCR.
a.

Human preadipocytes were purchased from Biowhittaker and either allowed to remain undifferentiated or subjected to differentiation. Human differentiated adipocytes were purchased from Zen Bio. RNA was prepared from these undifferentiated or differentiated human adipocytes and converted to cDNA. RT-PCR was then carried out in order to interrogate expression of RUP43 using the specific primers

5′-CTACCTGTACCTCGAAGTCTA-3′ (sense-primer;
SEQ ID NO:11)
and
5′-AGTGGCGGGCGCTGCTCAT-3′ (antisense-primer;
SEQ ID NO:12).

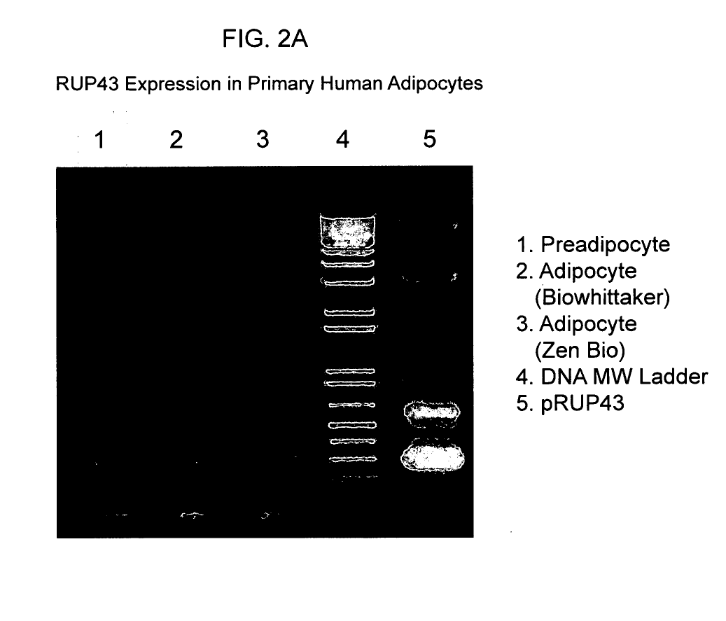

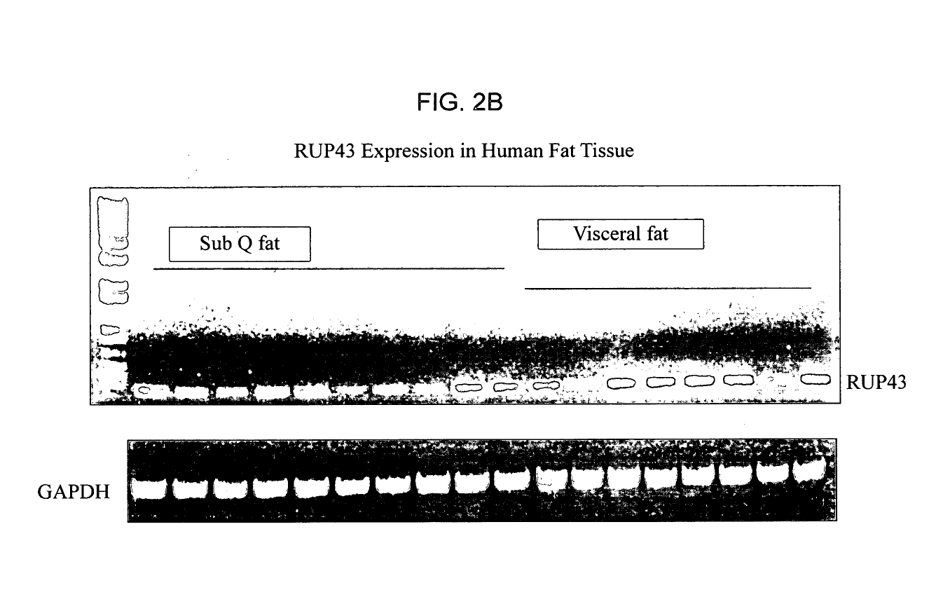

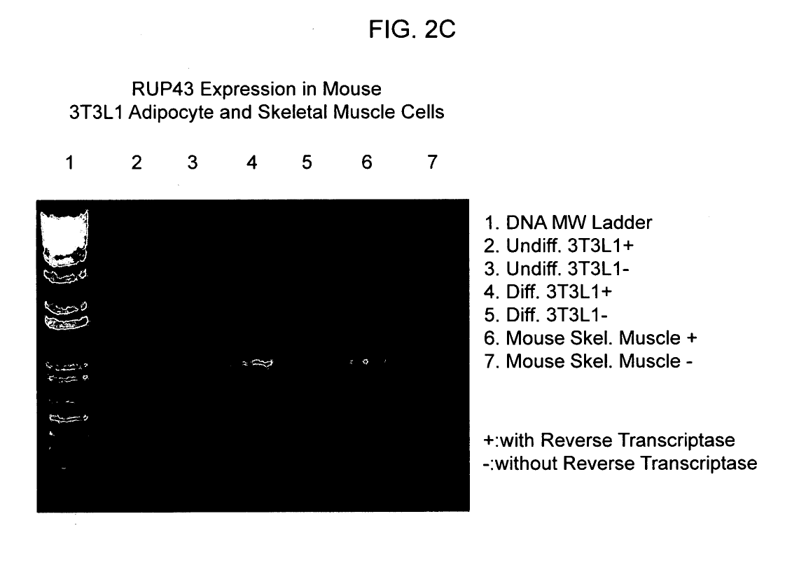

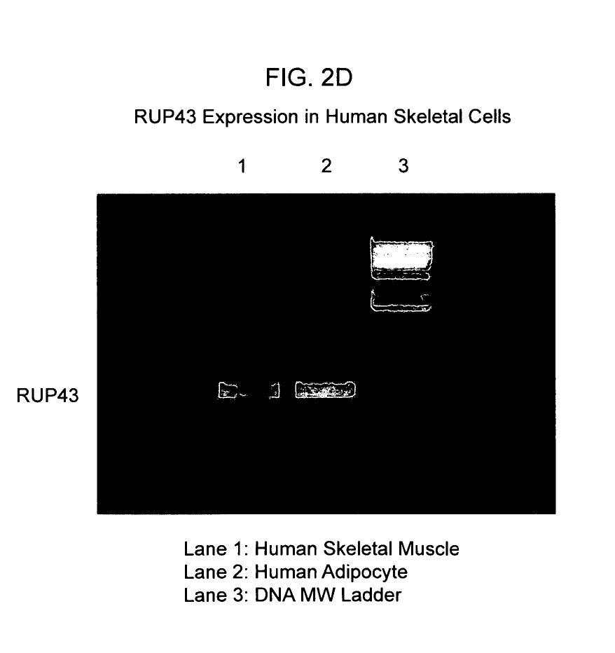

The cycle condition used was 94° C. for 2 min, 94° C. for 15 sec, 55° C. for 30 sec, and 72° C. for 1 min, with 35 cycles for the final three steps. RUP43 was found to be expressed endogenously by differentiated human adipocytes and to a lesser extent by human preadipocytes (FIG. 2A).
b.

Expression of RUP43 by human subcutaneous (“Sub Q”) and visceral fat was interrogated by RT-PCR as in [a] above. Subcutaneous fat samples were obtained from ten individuals with BMI ranging from 19 to 35. Visceral fat samples were obtained from eight individuals with BMI ranging from 19 to 45. RT-PCR of glyceraldehyde-3-phosphate dehydrogenase (GAPDH) was used to show comparable loading of samples. Human RUP43 was found to be expressed endogenously both in subcutaneous and in visceral fat (FIG. 2B).
c.

Mouse 3T3L1 cells were allowed to remain undifferentiated or were subjected to differentiation. RNA was prepared from undifferentiated 3T3L1 cells, from differentiated 3T3L1 cells, or from mouse skeletal muscle cells. Conversion of the RNA to cDNA was carried out either in the presence (“+”) or absence (“−”; negative control) of reverse transcriptase. RT-PCR was then carried out in order to interrogate expression of RUP43 using the specific primers

5′-TGAGCTGTCGGCCATrCCCAT-3′ (sense-primer, SEQ ID NO:13) and

5′-GATTGTCCCTCTTGGCTCTTC-3′ (antisense-primer, SEQ ID NO:14).

The cycle condition used was 94° C. for 2 min, 94° C. for 15 sec, 55° C. for 30 sec, and 72° C. for 1 min, with 35 cycles for the final three steps. RUP43 was found to be expressed by differentiated mouse 3T3L1 adipocytes and to a lesser extent by undifferentiated 3T3L1 adipocytes. RUP43 was also found to be endogenously expressed by mouse skeletal muscle cells (FIG. 2C).
d.

Human skeletal muscle cells were obtained from Cambrex. RNA was prepared from the skeletal muscle cells and converted to cDNA As a positive control, cDNA prepared as in [a] from human adipocytes obtained from Biowhittaker was used. RT-PCR was carried out as described in [a]. RUP43 was found to be endogenously expressed by skeletal muscle cells, and as previously shown in [a], by adipocytes (FIG. 2D).

### Example 12

Adipocyte Differentiation

Differentiation of Mouse 3T3L1 Cells

| | | |
|---|---|---|
| 3T3L1 Growth Medium | 1000 | ml |
| DMEM | 1000 | ml |
| 10% BCS | 100 | ml |
| L-glutamine, 200 mM | 10 | ml |
| P/S | 10 | ml |

| | | |
|---|---|---|
| 3T3L1 Regular Medium | 1000 | ml |
| DMEM | 1000 | ml |
| 10% FBS | 100 | ml |
| L-glutamine, 200 mM | 10 | ml |
| P/S | 10 | ml |

| | | |
|---|---|---|
| 3T3L1 Inducing Medium | 1000 | ml |
| DMEM | 1000 | ml |
| 10% FBS | 100 | ml |
| L-glutamine, 200 mM | 10 | ml |
| P/S | 10 | ml |
| Insulin (10 mg/ml) | 1 | ml |
| IBMax (10 mg/ml) | 11.1 | ml |
| Dexamethasone (10 mg/ml) | 328 | μl |

| | | |
|---|---|---|
| 3T3L1 Insulin Only Medium | 1000 | ml |
| DMEM | 1000 | ml |
| 100% FBS | 100 | ml |
| L-glutamine, 200 mM | 10 | ml |
| P/S | 10 | ml |
| Insulin (10 mg/ml) | 1 | ml |

DMEM: HYQ DEM/High glucose, SH30081.01, 500 ml. SH30081.02, 1000 ml.
BCS: Bovine Calf Serum, Hyclone SH 30073.03
FBS: Fetal Bovine Serum, Hyclone SH 30071.03
L-glutamine 200 mm, 100x. Hyclone SH40003-11
Penicillin-Streptomycin, 100 ml Hyclone SV30010
Trypsin, HYQ, 0.05% 1x, SH30236.01 100 ml.
HYQ DPBS/modified, 1x SH30028.02, 50 ml.

3T3L1 cells were seeded at 50% confluence such that the culture was fully confluent the next day. Two days after the cells have reached 100% confluence, inducing medium was added. Two to five days later, the cells were changed to insulin only medium Two to five days after induction, the cells were returned to regular medium for two days, completing the process of 3T3L1 differentiation to adipocytes.
b. Differentiation of Human Preadipocytes

Human preadipocytes purchased from Cambrex were seeded in a 24-well plate at 1×10⁶ cells/plate. After two days, when the cells reached 100% confluence, inducing medium purchased from Cambrex was added. The cells were cultured in inducing medium for ten days, thereby completing the process of differentiation of primary human preadipocytes to adipocytes.

### Example 13

DIFFERENTIATION OF HUMAN SKELETAL MUSCLE CELLS

Human primary undifferentiated skeletal muscle cells cultured in SKGM-2 medium purchased from Cambrex. When the skeletal myoblast culture achieved 50-70% confluence, the SKGM-2 medium was removed, and fusion medium (DMEM-F12 supplemented with 2% horse serum) was added.

Culture of the cells in the fusion medium was continued for 7 days (with replacement of the fusion medium every other day) or until myotubes were observed throughout the culture.

The resulting differentiated cultures were observed to contain multinucleated (more than 3 nuclei) myotubes.

If the myotubes were to be used in assays that required an extended period of time in culture, the fusion medium was removed and replaced with SKGM-2 medium For best performance, the SKGM-2 medium was replaced every other day to maintain the culture for 2-3 weeks. Myotube cultures were best used by 2 weeks post differentiation.

### Example 14

Endogenous RUP43 Couples to Gs

Gs coupling by RUP43 was interrogated by comparing the intracellular level of cAMP in HEK293 cells transfected with endogenous human, mouse, or rat RUP43 with mock-transfected HEK293 cells (“pCMV”). Determination of intracellular cAMP level was carried out by cyclase assay, using the Perkin Elmer Flashplate Kit (SMP004B) with ¹²⁵I as the tracer NEX130) essentially as per manufacturer's instructions.

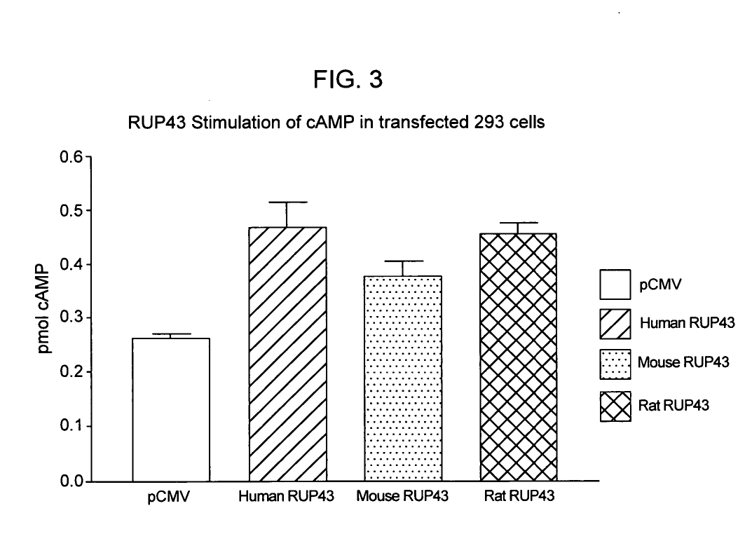

HEK 293 cells were plated at a density of 1.2×10⁷ and allowed to adhere overnight. The HEK293 cells were then transfected with pCMV alone or with pCMV containing polynucleotide encoding endogenous human, mouse, or rat RUP43, using lipofectamine (120 μg per 15 cm dish). The transfected cells were allowed to recover overnight For the assay, the transfected cells were harvested and added to a flashplate well at a final cell count of 1×10⁶ cells. They were allowed to adhere, then subjected to the tracer for two hours. All the wells were then aspirated and the plate was read using the microplate reader (Wallac 1450 microbeta counter). It was found that the intracellular level of RUP43-transfected HEK293 cells was significantly greater than that of mock-transfected cells, indicating that RUP43 manifests a detectable level of constitutive Gs coupling (FIG. 3).

### Example 15

Identification of Compound 1 as an Agonist of RUP43

Materials

HEK 293 cells obtained from ATCC were used for all assays. Culture media consisted of 90 mls of DMEM supplemented with 10% fetal bovine serum (Gibco, BRL). Cyclic AMP measurements were determined using the Adenylyl Cyclase Activation Flashplate® Assay with direct cAMP [¹²⁵I] Detection system.

Transient Transfection and Whole-Cell Cyclase Flashplate Assay

HEK 293 cells (5×10⁵ cells/ml) were plated in 15 cm dishes. The next day, cells were transfected using FuGENE 6 reagent (Roche Applied Science) as manufacturer suggested. Briefly, transfection mix consisting of OptiMEM (Gibco, BRL) and FuGENE 6 reagent were mixed together and allow to incubate for 5 minutes at room temperature. Transfection reagent was added drop-wise into a separate tube containing 2 μg of endogenous human RUP43 receptor plasmid (Transfected) or 2 μg of empty pCMV vector (Mock) and allowed to incubate for 15 minutes at room temperature. The DNA/transfection mixture was added drop-wise to each perspective plate and incubated over night in a humidified incubator maintained at 37° C., 95/5% O₂/CO₂. The next day, the media was replaced with normal growth media and the cells were incubated over night.

On Day 3, the cells were rinsed once with PBS and dislodged from the plate using a nonenzymatic cell-dissociation buffer (Gibco,BRL) and resuspended in assay stimulation buffer (Perkin Elmer) at a density of 2×10⁶ cell/ml for measurement of cAMP. Compound 1 or vehicle was serially diluted in stimulation buffer at 2× the desired final concentration. Compound 1 and vehicle (50 μl/well) were added to the perspective wells of a 96-well Flashplate® Perkin Elmer). Transfected or Mock cells were aliquot to each well (50 μL/well) and allowed to incubate at room temperature on a plate form shaker for 1 hour. Detection buffer® (Perkin Elmer, 100 μL) was added to each well and incubated for 2 hours at room temperature with mild agitation. At the end of the 2 hour incubation, the plate was aspirated and cAMP levels were determined using Wallac 1450 microbeta counter.

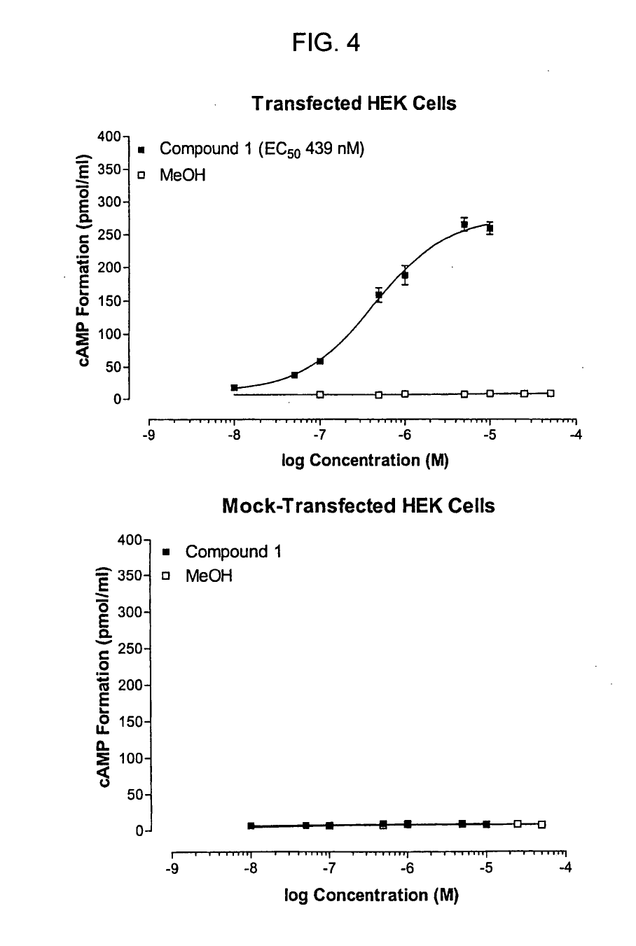

It was found that Compound 1, in a dose dependent manner, led to an increase in intracellular cAMP specifically in HEK293 cells transfected with endogenous human RUP43 and not in Mock transfected cells, identifying Compound 1 to be an agonist of RUP43 (FIG. 4).

### Example 16

Identification of Compound 2 as an Agonist of RUP43

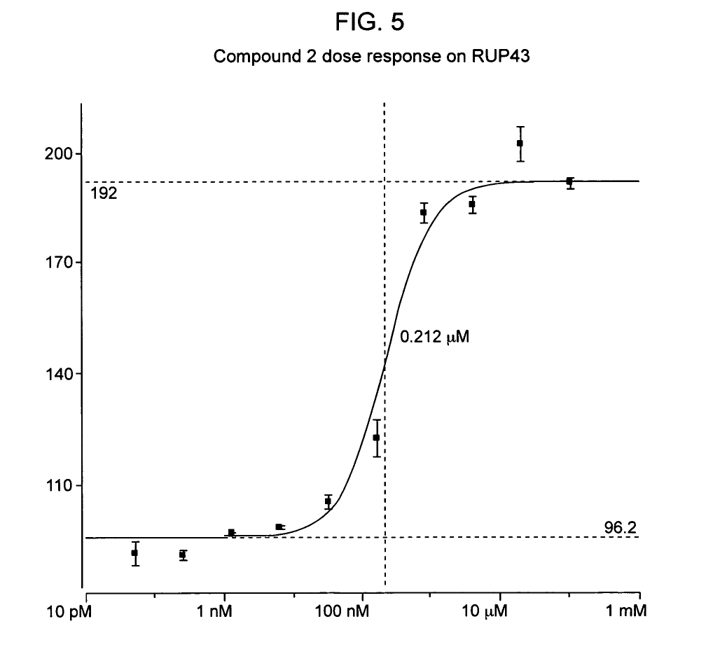

Using melanophore technology (example 10, supra), Compound 2 was found to be an agonist of endogenous human RUP43 (FIG. 5). Briefly, melanophore cells were harvested from confluent flasks (T-185 cm² flask) using Trypsin (0.7X), and transfected by electroporation. Polynucleotide encoding endogenous human RUP43 (30 μg) was used for transfection of melanophores. After electroporation, cells were preplated in flasks approximately 3-4 hours to rid of non-viable cells and debris. Upon completion, flasks were subsequently trypsinized and plated in triplicate onto 384 well poly-D-lysine coated plates for assay. Forty-eight hours post-transfection, assay plates were read in a spectrophotometer (absorbance T₀). Cells were then incubated for one hour with serially diluted Compound #2 (100 uM-51.2 pM, 5-fold dilutions, 0.5% DMSO final) and read again (absorbance Too). Triplicate absorbance data were analyzed and depicted as percent control response as compared to positive control wells (200) and negative control wells (100). Curve height was approximately 92% of control with an EC50=0.212uM.

### Example 17

In Vitro Glucose Uptake Assay

The in vitro glucose uptake assay was carried out as described here.

Buffers and Reagents:

Starvation medium: DMEM/high glucose with 0.5% BSA.

KRPH buffer 5 mM NaHPO₄, pH 7.4 (Make KRPH buffer fresh each time)
20 mM Hepes, pH 7.4
1 mM MgSO₄
1 mM CaCl₂
136 mM NaCl p1 4.7 mM KCl
1% BSA

2-Deoxyglucose (DOG): Stock 100 mM: 16.4 mg/ml in water (store at4 C 1-2 weeks).

For each well, add 1 μl containing 1 μCi [³H]-2-DOG and 1 μl cold stock 2-DOG and 2 ml KRPH.

Cytochalasin B (CytoB): Stock (10 mM in 95% ethanol): Keep at −20° C.

Use CytoB at 10 μM final to block carrier-mediated uptake. Also use this concentration at the end to stop the reaction. The stop buffer is PBS plus 10 μM CytoB (“PBS”).

1% Triton-X: This is the solubiliztion buffer.

Cells are plated in 24-well plates.

Procedure:

1. Starve cells for at least 2 hrs.

2. Wash cells 2 times with KRPH buffer and add 2 mls of KRPH to the well.

3. Treat cells with insulin and/or with test compound, or with vehicle (Control), for 20 min.

4. After 20 min, aspirate the buffer from the well and immediately add 1 ml of KRPH buffer plus 2-DOG. For CytoB-treated cells, add CytoB 5 min before uptake assay.

5. After 4 min, aspirate the buffer from the well and add 3 mls of cold PBS. After completing the assay, wash the cells in each well 2 times with cold PBS. Aspirate the Stop PBS completely, and then add 700 ul of 1% Triton X. Place in 37° C. incubator for 30 min

6. Count CPM in total lysate. Calculate CPM/well.

Subtract the CytoB value from the value obtained for each of cells treated with insulin and/or with test compound and cells treated with vehicle (Control).

### Example 18

Compound 2 Stimulates Glucose Uptake in Mouse 3T3L1 Adipocytes

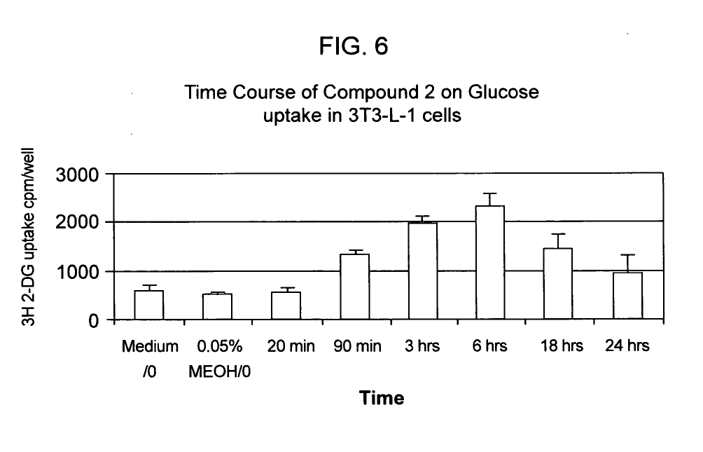

Differentiated mouse 3T3L1 adipocytes were treated with 50 μM Compound 2 for various times, after which glucose uptake was determined according to Example 17. From FIG. 6, it is apparent that Compound 2 stimulated glucose uptake in 3T3L1 adipocytes. The results indicate that RUP43 agonist is an attractive candidate for modulating glucose level in hyperglycemia that insulin fails to control. The rapid time course of the stimulated glucose uptake suggests that RUP43 agonist may provide a more rapid therapeutic effect than do currently available drugs for lowering blood glucose concentration.

### Example 19

Compound 2-Enhances Insulin-Stimulated Glucose Uptake in Mouse 3T3L1 Adipocytes

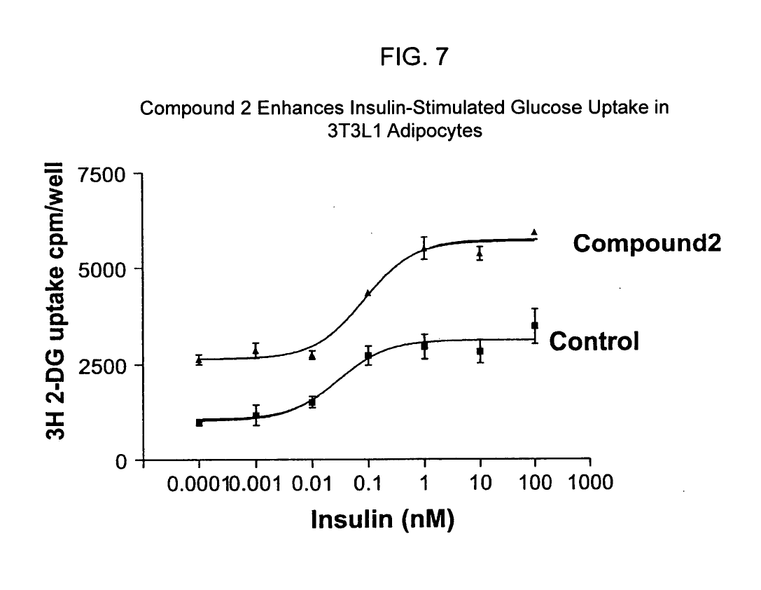

Differentiated mouse 3T3L1 adipocytes were treated with (“Compound 2”) or without (“Control”) Compound 2 in serum-free medium for 3 hr. The 3T3L1 cells were then washed with fresh KRPH buffer twice and treated with various concentrations of insulin for 20 min. After treatment with insulin, glucose uptake was determined according to Example 17. From FIG. 7, it is apparent that Compound 2 enhanced insulin-stimulated glucose uptake in 3T3L1 adipocytes. The results indicate that RUP43 agonist can increase insulin efficacy, thereby lowering the concentration of insulin required to achieve maximal glucose uptake.

### Example 20

Compound 2 Stimulates Glucose Uptake in Human Primary Human Adipocytes

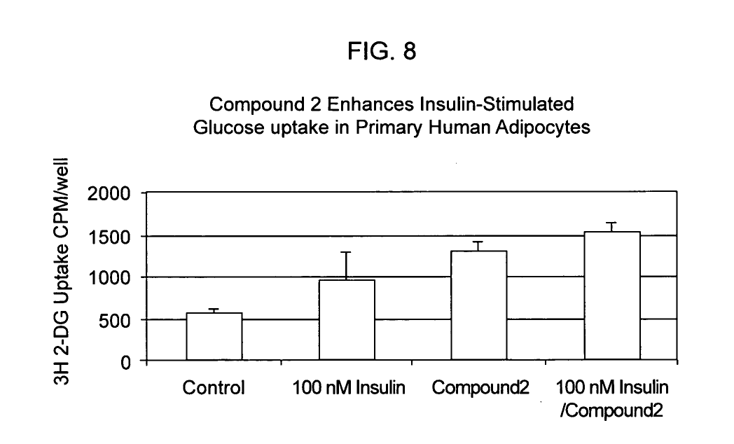

Human preadipocytes (Cambrex) were differentiated into adipocytes. The differentiated primary human adipocytes were treated with or without 50 μM Compound 2 for 3 hr in serum-free medium. The human adipocytes were then washed with fresh KRPH buffer twice and treated with or without 100 nM insulin for 20 min. After treatment with or without insulin, glucose uptake was determined according to Example 17. From FIG. 8, it is apparent that Compound 2 stimulated glucose uptake in primary human adipocytes. From FIG. 8 it is also apparent that Compound 2 enhanced insulin-stimulated glucose uptake in primary human adipocytes. Significantly, as was observed for the mouse 3n3L1 cells, RUP43 agonist can stimulate glucose uptake in primary human adipocytes in the absence of insulin, and the level of RUP43-stimulated glucose uptake is comparable to the level of insulin-stimulated glucose uptake.

### Example 21

Compound 2 Stimulates Glucose Uptake in Rat L6 Myoblast Cells

Rat skeletal muscle L6 myoblast cells were obtained from ATCC and grown in 24-well plates to confluence.
a.

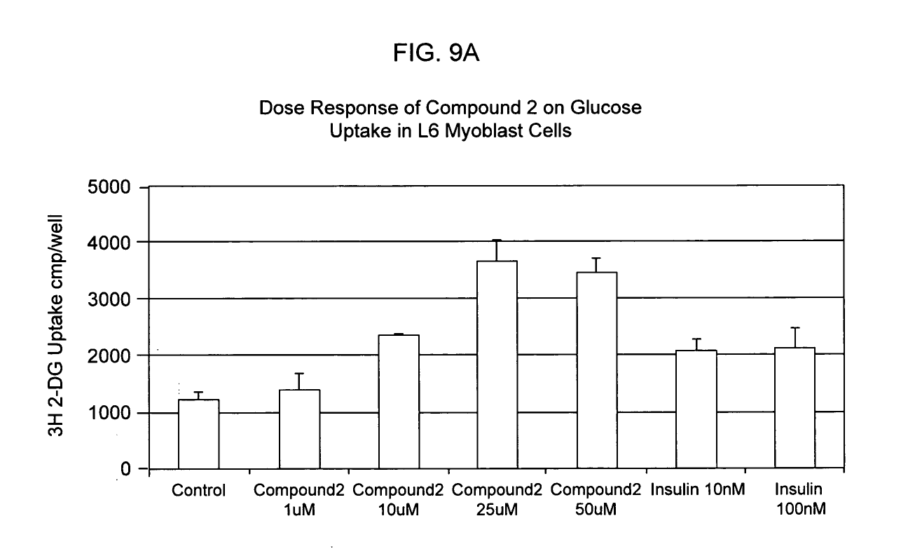

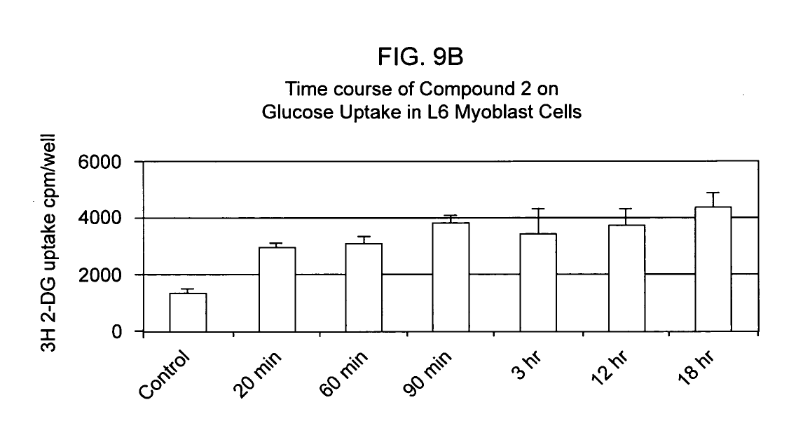

Confluent L6 cells were treated with or without various concentrations of Compound 2 in serum-free medium for 3 hr. The L6 cells were then washed twice with KRPH buffer. L6 cells which had been treated with Compound 2 were incubated with KRPH buffer for 20 min; L6 cells which had not been treated with Compound 2. were treated with 10 nM or 100 nM insulin for 20 min. After treatment with or without insulin, glucose uptake was determined according to Example 17. From FIG. 9A, it is apparent that Compound 2 stimulated glucose uptake in rat L6 myoblast cells. RUP43 agonist stimulated greater glucose uptake in rat L6 myoblast than did insulin. As skeletal muscle cells are responsible for 80% of glucose disposal in vivo, the results obtained indicate that RUP43 agonist is an attractive candidate for providing better glucose disposal in vivo than does insulin.
b.

Confluent L6 myoblast cells were treated with 50 μM Compound 2 for various times. At the end of each treatment period, glucose uptake was determined according to Example 17. The results indicate that RUP43 agonist can stimulate glucose uptake in skeletal muscle cells within 20 min, a timeframe similar to that of insulin (FIG. 9B). The results indicate that RUP43 agonist is an attractive candidate for regulating glucose level in vivo within a short period of time comparable to that of insulin.

### Example 22

Compound 2 Enhances Insulin-Stimulated Glucose Uptake in Rat L6 Myoblast Cells

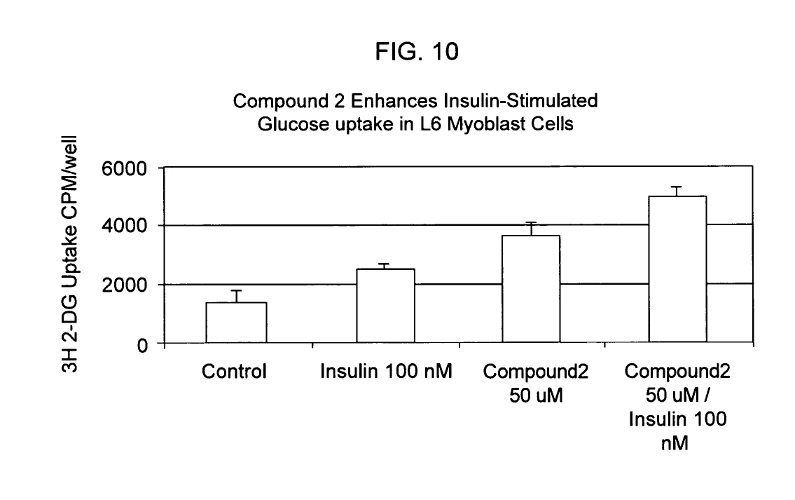

Confluent rat L6 myoblast cells were treated with or without 50 μM Compound 2 for 3 hr in serum-free medium. The L6 cells were then washed twice with fresh KRPH buffer. The L6 cells were then treated with or without 100 nM insulin for 20 min. After treatment with or without insulin, glucose uptake was determined according to Example 17. From FIG. 10, it is apparent that, analogous to what was observed for adipocytes, Compound 2 enhances insulin-stimulated glucose uptake in rat L6 myoblast cells. The results further indicate that RUP43 agonist can increase insulin efficacy, thereby lowering the concentration of insulin required to achieve mammal glucose uptake. RUP43 therefore represents an attractive therapeutic option for an individual suffering from hyperinsulinemia-caused problems relating to insulin resistance.

### Example 23

Compound 2 Stimulates Glucose Uptake in Primary Human Skeletal Muscle Cells

Primary human skeletal muscle cells obtained from Cambrex were grown to 50% confluence and then differentiated on culture with inducing medium for 5 to 7 days. The differentiated primary human skeletal muscle cells were then transferred to growth medium for 7 to 10 days.

Differentiated human skeletal muscle cells were treated with or without various concentrations of Compound 2 in serum-free medium for 3 hr. After treatment with or without Compound 2, the cells were washed twice with fresh KRPH buffer. Cells which had been treated with Compound 2 were then incubated with KRPH buffer for 20 min; cells which had not been treated with Compound 2 were incubated with 10 nM or 100 nM insulin for 20 min. After treatment with or without insulin, glucose uptake was determined according to Example 17. From FIG. 1A, it is apparent that RUP43 agonist can regulate glucose uptake in human skeletal muscle cells in the absence of insulin and more effectively than insulin. The results obtained indicate that RUP43 agonist is efficacious at stimulating glucose uptake in human skeletal muscle cells, where 80% of glucose disposal takes place. The results obtained indicate that RUP43 agonist is an attractive candidate for controlling glucose level in hyperglycemia refractory to insulin action
b.

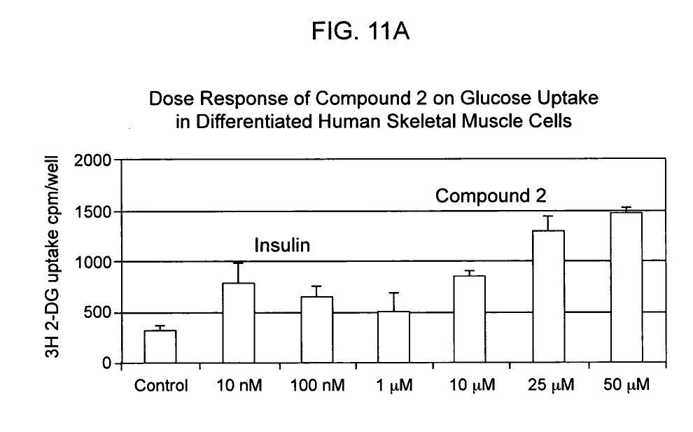

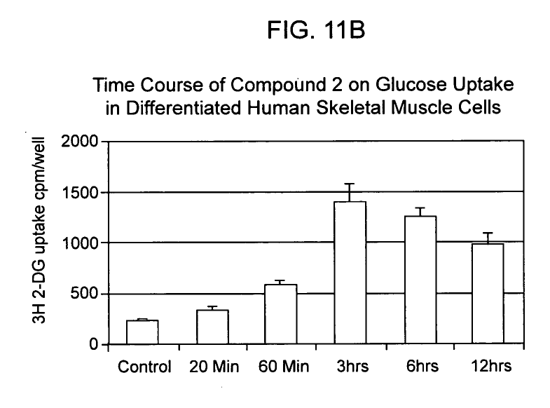

Differentiated human skeletal muscle cells were treated with 50 μM Compound 2 for various time periods. At the end of each treatment period, glucose uptake was determined according to Example 17. The results obtained are presented in FIG. 11B. The rapid stimulation of glucose uptake in human skeletal muscle cells observed for RUP43 agonist indicate RUP43 agonist to be an attractive candidate for regulating glucose level in vivo directly and within a short period of time.

### Example 24

Oral Bioavailability

Physicochemico analytical approaches for directly assessing oral bioavailability are well known to those of ordinary skill in the art and may be used [see, e.g., without limitation: Wong PC et al., Cardiovasc Drug Rev (2002) 20:137-52; and Buchan P et al., Headache (2002) Suppl 2:S54-62; the disclosure of each of which is hereby incorporated by reference in its entirety]. By way of further illustration and not limitation, said alternative analytical approaches may comprise liquid chromatography-tandem mass spectrometry [Chavez-Eng CM et al., J ChromatogrB Analyt Technol Biomed Life Sci (2002) 767:117-29; Jetter A et al., Clin Pharmacol Ther (2002) 71:21-9; Zimmerman J J et al., 3 Clin Pharmacol (1999) 39:1155-61; and Barrish A et al., Rapid Commun Mass Spectrom (1996) 10:1033-7; the disclosure of each of which is hereby incorporated by reference in its entirely]. Recently, positron emission tomography (PET) has been successfully used to obtain direct measurements of drug distribution, including oral bioavailability, in the mammalian body following oral administration of the drug [Gulyas et al., Eur J Nucl Med Mol Imaging (2002) 29:1031-8; the disclosure of which is hereby incorporated by reference in its entirety].

Alternatively, based upon the in vivo data developed, as for example by way of illustration and not limitation, through the mouse model of Example 26. The modulator is administered by oral gavage at doses ranging from 0.1 mg kg to 100 mg kg. The effect of the modulator is shown to be dose-dependent and comparable to the effect after intraperitoneal administration, wherein the effect is reduction of blood glucose concentration (Example 26). The dose of modulator required to achieve half-maximal reduction of beneficial effect through oral administration is compared to the dose of modulator required to achieve half-maximal reduction of beneficial effect through intraperitoneal administration. By way of illustration, if said oral dose is twice said intraperitoneal dose, then the oral bioavailability of the modulator is taken to be 50%. More generally, if said oral dose is 8 mg kg⁻¹ and said intraperitoneal dose is p mg kg⁻¹, then the oral bioavailability of the modulator as a percentage is taken to be [(p/θ)×100].

### Example 25

Blood Brain Barrier Model

The ability of a compound of the invention to cross the blood-brain barrier can be determined using brain-derived cells. One method that is envisioned, by way of illustration and not limitation, is to use the blood/brain barrier model of Dehouck et al. [3 Neurochem (1990) 54:1798-801; hereby incorporated by reference in its entirety] that uses a co-culture of brain capillary endothelial cells and astrocytes.

Bovine capillary endothelial (BBCE) cells are isolated and characterized as described by Meresse et al. [3 Neurochem (1989) 53:1363-1371; hereby incorporated by reference in its entirety]. In brief, after isolation by mechanical homogenization from one hemisphere of bovine brain, microvessels are seeded onto dishes coated with an extracellular matrix secreted by bovine corneal endothelial cells. Five days after seeding, the first endothelial cells migrate out from the capillaries and begin to form microcolonies. When the colonies are sufficiently large, the five largest islands are trypsinized and seeded onto 35-mm diameter gelatin-coated dishes (one clone per dish) in the presence of Dulbecco's modified Eagle's medium (DUMM) supplemented with 15% calf serum (Seromed), 3 mM glutamine, 50 μg/ml of gentamicin, 2.5 μg/ml of amphotericin B (Fungizone), and bovine fibroblast growth factor (1 ng/ml added every other day). Endothelial cells from one 35-mm-diameter dish are harvested at confluence and seeded onto 60-mm-diameter gelatin-coated dishes. After 6-8 days, confluent cells are subcultured at the split ratio of 1:20. Cells at the third passage (˜100 dishes) are stored in liquid nitrogen.

Primary cultures of astrocytes are made from newborn rat cerebral cortex. After the meninges have been cleaned off, the brain tissue is forced gently through a nylon sieve as described by Booher and Sensenbrenner [Neurobiology (1972) 2:97-105; hereby incorporated by reference in its entirety]. DMM supplemented with 10% fetal calf serum (Seromed), 2 mM glutamine, and 50 μg/ml of gentamicin is used for the dissociation of cerebral tissue and development of astrocytes.

Culture plate inserts (Millicell-CM; pore size, 0.4 μM; diameter, 30 mm; Millipore) are coated on both sides with rat tail collagen prepared by a modification of the method of Bornstein [Lab Invest (1958) 7:134-139; hereby incorporated by reference in its entirety].

Astrocytes are plated at a concentration of 2.5×10⁵ cells/ml on the bottom side using the filter upside down. After 8 days, filters are properly positioned, and the medium is changed twice a week. Three weeks after seeding, cultures of astrocytes become stabilized. Then, BBCE cells, frozen at passage 3, are recultured on a 60-mm-diameter gelatin-coated dish. Confluent cells are trypsinized and plated on the upper side of the filtures at a concentration of 4×10⁵ cells. The medium used for the coculture is DMEM supplemented with 15% calf serum 2 mM glutamine, 50 μg/ml of gentamicin, and 1 ng/ml of bovine fibroblast growth factor added every other day. Under these conditions, BBCE cells form a confluent monolayer in 8 days.

Culture plates are set into six-well plates with 2 ml of buffer added to the upper chamber and 2 ml added to the plate containing the inserts. The six-well plates are placed in a shaking water bath at 37° C. The compound of the invention is added to the upper chamber, and 100 μl is removed from the lower chamber at various time points. In certain embodiments, the test compound is radiolabeled. In certain embodiments, the radiolabel is ³H or ¹⁴C In some embodiments, the final time point is about 20 min, about 30 min, about 40 min, about 50 min, about 60 min, about 70 min, about 80 min or about 90 min. The percentage of total test compound present in the lower chamber at the various time points is determined. Leucine is used as a permeability positive control. Insulin is used as a permeability negative control.

In certain embodiments, a determination of at least about 10%, at least about 20%, at least about 30%, at least about 40%, at least about 50%, at least about 60%, at least about 70%, at least about 80% or at least about 90% of the compound of the invention in the lower chamber at the final time point is indicative of the compound of the invention being able to cross the blood-brain barrier.

### Example 26

Iv vivo Effects OF RUP43 Agonists on Glucose Homeostasis in Rats

A. Oral Glucose Tolerance Test (oGTT) in Rats.

Male Zucker diabetic fatty (ZDF) rats (Charles River) at age of 10 weeks are fasted for 18 hours and randomly grouped (n=11) to receive a RUP43 agonist at various doses, or with control rosiglitazone (RSG, 10 mg/kg) known to increase insulin sensitivity. The RUP43 agonist is delivered intraperitoneally. RSG is delivered intaaperitoneally. A preferred dose of RUP43 agonist is 0.1-100 mg/kg. Other preferred dose is selected from the group consisting of: 0.1 mg/kg, 0.3 mg&g, 1.0 mg/kg, 3.0 mg/kg, 10 mg/kg, 30 mg/kg and 100 mg/kg. The placebo group is administered vehicle.

Thirty minutes after administration of test compound and control RSG, rats are administered orally with dextrose at 2 g/kg dose. Levels of blood glucose are determined at various time points using Glucometer Elite XL (Bayer). Taking the time of dextrose administration to be “0 min”, exemplary time points are −30 min, 0 min, 30 min. 60 min, 90 min and 120 min The mean glucose concentration is averaged from eleven animals in each treatment group. These results can demonstrate that RUP43 agonist lowers blood glucose in a dose-dependent manner in rats after challenge with glucose.

Alternatively, the oral glucose tolerance test as described here is carried out in the rats immediately following seven daily injections of RUP43 agonist, RSG, or vehicle.

It is expressly contemplated that the oral glucose tolerance test described here may also be carried out in a different animal, for example in mouse or in rabbit.

B. Acute Response of ZDF Rats to RUP43 Agonist.

Male Zucker diabetic fatty (ZDF) rats (Charles River) at age of 10 weeks are randomly grouped (n=6) to receive vehicle (intraperitoneally), RUP43 agonist (intraperitoneally), or rosiglitazone (RSG, 10 mg/kg, intraperitoneally). A preferred dose of RUP43 agonist is 0.1-100 mg/kg. Other preferred dose is selected from the group-consisting of: 0.1 mg/kg, 0.3 mg/kg, 1.0 mg/kg, 3.0 mg/kg, 10 mg/kg, 30 mg/kg and 100 mg/kg. After compound administration, food is removed and blood glucose levels are determined at various times. Exemplary times of glucose level determination are 0 hr, 1 hr, 2 hr, 3 hr and 4 hr, and then daily for up to a week. Reduction in blood glucose at each time point is expressed as percentage of original glucose levels, averaged from six animals for each group. These animals have blood glucose levels (fed state) of 300-400 mg/dl, significantly higher than non-diabetic wild type animals. Treatment with RUP43 agonist or RSG can be shown to significantly reduce glucose levels compared to vehicle control. These data can demonstrate that RUP43 agonist has efficacy in improving glucose homeostasis in diabetic animals.

Alternatively, the rats are injected daily for seven days with RUP43 agonist RSG, or vehicle immediately before blood glucose level is determined daily for seven days.

It is expressly contemplated that the acute response test described here may also be carried out in a different animal for example in mouse or in rabbit.

### Example 27

Synthesis of Compounds of the Invention

### Example 27A

### 2-{1-[2-(2-Chloro-phenyl)-acetyl]-piperidin-4-yl)-thiazole-4-carboylic acid methyl

### (2-methyl-4,5,6,7-tetrahydro-2H-indazol-3-yl)amide

### 2-Piperidin-4yl-thiazole-4-carboxylic acid ethyl ester dihydrobromide salts

A solution of tert-butyl-4-(aminocarbothioyl)tetrahydropyridine-1(2H)-carboxylate (2.0 g, 8.2 mmol) and ethyl bromopyruvate (1.6 g, 8.2 mmol) in 30 mL of EtOH was stirred at 80° C. for 4h. Afterwards, the mixture cooled to room temperature and then charged with 48% HBr (1.0 mL, 14 mmol. The reaction mixture was allowed to stir an additional 1 h, and then concentrated to an oily solid. Trituration with diethyl ether afforded 3.0 g (91%) of a tan solid. ¹H NMR (400 Liz, DMSO-d₆) δ 9.02 (br s, 1 H), 8.77 (br s, 1 H), 8.46 (s, 1 H), 7.01 (br s, 1 H),4.29 (q, J=7.1 Hz,2 H), 3.44-3.33 (m, 3 H), 3.-2 (q, J=11.7 Hz 2 H), 2.19 (d, J=13.2 Hz, 2 H), 1.97-1.88 (m, 2 H), 1.29 (t, J=7.0 Hz, 3 H). MS calculated for C₁₁H₁₆N₂O₂S+H: 241, observed: 241.

### 2-{1-[2-(2-Chloro-phenyl)-acetyl]-piperidin-4-yl)-thiazole-4-carboxylic acid ethyl ester

A solution of 2-piperidin-4-yl-thiazole-4-carboxylic acid ethyl ester dihydrobromide salts (1.0 g, 3.2 mmol), 2-chlorophenyl acetic acid (0.55 g, 3.2 mmol), O-(7-azabenzotriazol-1-yl)N,N′,N′-tetramethyluronium hexafluorophosphate (1.4 g, 3.5 mmol), and diiospropylethylamine (3.0 mL, 17 mmol) in 30 mL of CH₂Cl₂ was stirred at 40° C. for 8h Afterwards, the crude mixture was diluted with 30 mL of CH₂Cl₂ and washed with 1 M citric acid (3×50 mL), saturated aqueous NaHCO₃ (1×30 mL), and saturated aqueous NaCl (1×30 mL). The resulting organic layer was dried over MgSO₄, filtered, and concentrated to a brown oil. Purification on silica gel with EtOAc hexanes (3:1) afforded 0.94 g (75%) of a light brown oil ¹H NMR (400 MHz, CDCl₃) δ 8.08 (s, 1 H), 7.39 (dd, J=7.6, 1.6 Hz, 1 H7.32 (dd, J=7.2, 2.0 Hz, 1 H), 7.25-7.19 (m, 2 H), 4.76 (appar d, 1 H), 4.42 (q, J=7.2 Hz, 2 H), 3.99-3.95 (m, 1 H). 3.88 (d, AB pattern, J_{AB}=16.0 Hz, 1 H), 3.83 (d, AB pattern, J_{AB}=16.0 Hz, 1 H), 3.34 (tt, J=11.7, 3.7 Hz, 1 H), 3.21-3.14 (m, 1 H), 2.83-2.76 (m, 1 H), 2.20-2.16 (m, 2 H), 1.74 (qd, J=12.3, 4.1 HZ, 1 H), 1.63 qd, J=12.3, 4.0 Hz, 1 H), 1.40 (t, J=7.2 Hz, 3 H). MS calculated for C₁₉H₂₁CIN₂O₃S+H: 393.

### 2-{1-[2-(2-Chloro-phenyl)-acetyl]-piperidin-4-yl)-thiazole-4-carboxylic acid

A solution of 2-{1-[2-(2 chloro-phenyl)-acetyl]-piperidin-4-yl}-thiazole-4-carboxylic acid ethyl ester (0.94 g, 2.4 mmol) in 20 mL of MeOH was diluted with 1 M NaOH (20 mL, 20 mmol) and was allowed to stir at 60° C. for 4h. Afterwards, the crude mixture was concentrated to remove the MeOH solvent. The aqueous basic solution was then washed with CH₂Cl₂ (2×25 mL) and acidified with 5 M HCl to pH=1. The resulting aqueous acidic solution was extracted with CH₂Cl₂ (3×25 mL). The organic layers were combined, dried over MgSO₄, filtered, and concentrated to afford 0.51 g (58%) of a white foamy solid. ¹H NMR (400 MHz, DMSO-d₆) δ 12.96 (br s, 1 H), 8.36 (s, 1 H), 7.45-7.40 (m, 1 H), 7.33-7.25 (m, 3 H), 4.43 (d, J=13.2 Hz, 1 H), 4.06 (d, J=13.6 Hz, 1 H), 3.87 (d, AB pattern, J_{AB}=1.60 Hz, 1 H), 3.82 (d, AB pattern J_{AB}=16.0 Hz, 1 H), 3.38-3.31 (m, 1 H), 3.25 (appar t, J=11.8 Hz, 1 H), 2.79 (appar t, J=11.6Hz, 1 H),2.08 (t, J=10.6 Hz, 2H), 1.67 (qd, J=12.1, 3.7 Hz, 1 H), 1.53 (qd, J=12.2, 4.0 Hz, 1 H). MS calculated for C₁₇H₁₇CIN₂O₃S+H: 365, observed: 365.

### 2-{1-[2-(2-Chloro-phenyl)-acetyl]-piperidin-4-yl}-thiazole-4-carboxylic acid methyl-(2-methyl-4,5,6,7-tetrahydro-2H-indazol-3-yl)-amide dihydrochloride salts

A solution of N,1-dimethyl-4,5,6,7-tetrahydro-1H-indazol-3-amine (23 mg, 0.14 mmol), O-7-azabenzotriazol-1-1-yl)-N,N,N′,N′-tetramethyluronium hexafluorophosphate (75 mg, 0.20 mmol, and 2-{1-[2-(2-Chloro-phenyl)-acetyl]-piperidin-4-yl}-thiazole-4-carboxylic acid (50 mg, 0.14 mmol) in 10 mL of CH₂Cl₂ was stirred at 40° C. for 8h. Afterwards, the crude mixture was diluted with 20 mL of CH₂Cl₂ and washed with 1 M citric acid (3×30 mL), saturated aqueous NaHCO3 (1×30 mL), and saturated aqueous NaCl (1×30 mL). The resulting organic layer was dried over MgSO₄, filtered, and concentrated to a yellow oil. Purification by gradient HPLC (acetonitrile-water with 0.1% TFA) and converting to dihydrochloride salts afforded 56 mg (70%) of a white solid. ¹H NMR (400 MHz, CD₃OD) δ 8.28 (d, J=8.8 Hz, 1 H), 7.43-7.41 (m, 1 H), 7.30-7.26 (m, 3 H), 4.50 (t, J=11.8 Hz, 1 H), 4.094.03 (m, 1 H), 3.98-3.91 (m, 2 H), 3.83 (d, J=12.8 Hz, 3 H), 337 (s, 3 H),323.20 (m, 1 H), 2.89-2.82 (m, 1 H), 2.83-2.66 (m, 2 H), 2.47-2.41 (m 1 H), 2.03-1.88 (m,4 H), 1.72-1.60 (m, 3 H), 1.46-1.26 (m, 3 H). MS calculated for C₂₆H₃₀CIN₅O₂S+H: 512, observed: 512.

### Example 27B

### 2-(2-Chloro-phenyl)-1-{4-[4-(3,4-dihydro-2H-quinoline-1-carbonyl)-thiazol-2-yl]-piperidin-1-yl}-ethanone

By the same general procedure as in Example 29A, 2-(2-Chloro-phenyl)-1-{4-[4-3,dihydro-2H-quinoline-1-carbonyl)-thiazol-2-yl]-piperidin-1-yl}-ethanone was obtained from 1,2,3,4-tetrahydroquinoline as a yellow solid ¹H NMR (400 MHz, CD₃OD) δ 7.78 (s, 1 H), 7.42-7.40 (m, 1 H), 7.30-7.24 (m, 3 H), 7.18 (d, J=7.6 Hz, 1 H), 7.03 (t, J=7.4 Hz, 1 H), 6.91 (t, J=7.6 Hz, 1 H), 6.72 (br s, 1 H), 4.28 (d, J=12.8 Hz, 1 H), 3.94-3.81 (m, 5 H), 3.30-3.20 (m, 2 H), 2.98-2.91 (m, 1 H), 2.83 (t, J=6.4 Hz, 2 H), 2.04 (quintet, J=6.6 Hz, 2 H), 1.99-1.93 (br m, 2 H), 1.60-1.51 (m, 2 H). MS calculated for CH₂₅H₂₆CIN₃O₂S+H: 480, observed. 480.

### Example 27C

### 2-{1-[2-2-Fluoro-phenyl)-acetyl]-piperidin-4yl}-thiazole-4-carboxylic acid methyl-(2-methyl-4,5,6,7-tetrahydro-2H-indazol-3-yl)-amide

By the same general procedure as in Example 29A, 2-{1-[2-(2-fluoro-phenyl)-acetyl]-piperidin-4-yl}-thiazole-4-carboxylic acid methyl-(2-methyl-4,5,6,7-tetrahydro-2H-indazol3-yl)-amide was obtained from 2-{1-[2-(2-fluoro-phenyl)acetyl]-piperidin-4-yl}-thiazole-4-carboxylic acid as a white solid. ¹H NMR (400 MHz, CDCl₃) δ 7.71 (d, J=10.4 Hz, 1 H), 7.30 (t, J=7.6 Hz, 1 H), 7.26-7.22 (m, 1 H), 7.11 (t, J=7.4 Hz, 1 H), 7.05 (t, J=9.0 Hz, 1 H), 4.47 (appar t, J=13.6 Hz, 1 H), 3.90-3.79 (m, 1 H), 3.74 (s, 2 H), 3.63 (d, J=4.4 Hz, 3 H), 3.32 (s, 3 H), 3.17-3.11 (m, 1 H), 3.04-2.95 (m, 1 H), 2.91-2.79 (m, 1 H), 2.61-2.43 (m, 2 H), 2.27 (dt, J=15.3, 5.7 Hz, 1 H), 1.99-1.88 (m, 3 H), 1.79-1.72 (m, 1 H), 1.64-1.38 (m, 5 H). MS calculated for C₂₆H₃₀FN₅O₂S+H: 496, observed: 496.

1-140. (canceled)

141. A method comprising:
(a) contacting a candidate compound with a G protein-coupled receptor comprising an amino acid sequence having at least 75% identity to SEQ ID NO:2, wherein said G protein-coupled receptor is present on a cell or isolated membrane thereof;
(b) determining the ability of the compound to inhibit or stimulate the G protein-coupled receptor; and
(c) determining if said compound modulates glucose metabolism.

142. The method of claim 141, wherein the method identifies modulators of glucose uptake in a mammal.

143. The method of claim 141, wherein the method identifies modulators of blood glucose concentration in a mammal.

144. The method of claim 141, wherein the method is for screening candidate compounds as pharmaceutical agents for preventing or treating a metabolic disorder selected from diabetes, impaired glucose tolerance, insulin resistance and hyperinsulinemia.

145. A process for making a compound which can inhibit or stimulate a G protein-coupled receptor having at least 75% identity to SEQ ID NO:2, comprising:
(a) identifying said compound using a method according to claim 141; and
(b) synthesizing the compound identified in (a).

146. A compound which can inhibit or stimulate a G protein-coupled receptor having at least 75% identity to SEQ ID NO:2, when identified using a method according to claim 141.

147. A compound of Formula (II).

148. A method of preparing a pharmaceutical composition comprising admixing a compound according to claim 146 or claim 147 and a pharmaceutically acceptable carrier.

149. A pharmaceutical composition comprising a compound according to claim 146 or claim 147 and a pharmaceutically acceptable carrier.

150. A method comprising administering to a mammal in need thereof a therapeutically effective amount of an agonist of the mammalian RUP43 G protein-coupled receptor.

151. The method of claim 150, wherein the method is for lowering blood glucose concentration.

152. The method of claim 150, wherein the method is for increasing glucose uptake.

153. The method of claim 150, wherein the method is for preventing or treating a metabolic disorder selected from the group consisting of diabetes, impaired glucose tolerance, insulin resistance, and hyperinsulinemia.

154. The method of claim 150, wherein the method is for preventing or treating a complication of an elevated blood glucose concentration selected from the group consisting of Syndrome X, atherosclerosis, atheromatous disease, heart disease, hypertension, stroke, neuropathy, retinopathy, nephropathy, and peripheral vascular disease.

155. A method comprising:
(a) contacting a test ligand with a G protein-coupled receptor comprising an amino acid sequence having at least 75% identity to SEQ ID NO:2,
wherein said G protein-coupled receptor is present on a cell or isolated membrane thereof; and
1 (b) detecting a ligand bound to said G protein-coupled receptor.

156. An isolated adipocyte or skeletal muscle cell comprising a recombinant nucleic acid encoding a polypeptide comprising an amino acid sequence having at least 75% identity to SEQ ID NO:2.
